(19) United States
(12) Patent Application Publication
(10) Pub. No.: US 2026/0055151 A1
(43) Pub. Date: Feb. 26, 2026

(54) ANTIMICROBIAL KINOCIDIN COMPOSITIONS AND METHODS OF USE

(71) Applicant: Lundquist Institute for Biomedical Innovation at Harbor-UCLA Medical Center, Torrance, CA (US)

(72) Inventors: Nannette Y. Yount, San Juan Capistrano, CA (US); Michael R. Yeaman, Redondo Beach, CA (US)

(21) Appl. No.: 19/098,883

(22) Filed: Apr. 2, 2025

Related U.S. Application Data
(63) Continuation of application No. 17/821,669, filed on Aug. 23, 2022, now abandoned.
Application No. 17/821,669 is a continuation of application No. 17/718,233, filed on Apr. 11, 2022, now abandoned.
Application No. 17/718,233 is a continuation of application No. 17/079,277, filed on Oct. 23, 2020, now abandoned.
Application No. 17/079,277 is a continuation of application No. 16/448,499, filed on Jun. 21, 2019, now abandoned.
Application No. 16/448,499 is a continuation of application No. 16/121,425, filed on Sep. 4, 2018, now Pat. No. 10,329,336.
Application No. 16/121,425 is a continuation of application No. 15/226,643, filed on Aug. 2, 2016, now abandoned.
Application No. 15/226,643 is a continuation of application No. 12/947,793, filed on Nov. 16, 2010, now Pat. No. 9,428,566.
Application No. 12/947,793 is a division of application No. 12/438,923, now abandoned.
(60) Provisional application No. 60/815,491, filed on Jun. 20, 2006.

(51) Int. Cl. C07K 14/52; A61K 38/00; C07K 14/47

(52) U.S. Cl. CPC C07K 14/521; C07K 14/4723; A61K 38/00; Y02A 50/30

(57) ABSTRACT

The present invention provides novel kinocidin peptides comprising a C-terminal portion of a kinocidin, wherein the C-terminal portion encompasses an α-helical secondary structure and further displays antimicrobial activity. The kinocidin peptides of the invention are derived from and correspond to a C-terminal portion of a kinocidin that includes a γκ_{O} core and that can be a CXC, CC, or C class chemokine. Structural, physicochemical and functional properties of this novel class of antimicrobial peptides and amino acid sequences of particular kinocidin peptides are also disclosed. The invention also provides related antimicrobial methods.

### CROSS REFERENCE TO RELATED APPLICATIONS

This application is a continuation of U.S. application Ser. No. 17/821,669, filed Aug. 23, 2022, which is a continuation of U.S. application Ser. No. 17/718,233, filed Apr. 11, 2022, now abandoned, which is a continuation of U.S. application Ser. No. 17/079,277, filed Oct. 23, 2020, now abandoned, which is a continuation of U.S. application Ser. No. 16/448,499, filed Jun. 21, 2019, now abandoned, which is a continuation of U.S. application Ser. No. 16/121,425, filed Sep. 4, 2018, now U.S. Pat. No. 10,329,336, which is a continuation of U.S. application Ser. No. 15/226,643, filed Aug. 2, 2016, now abandoned, which is a continuation of U.S. application Ser. No. 12/947,793, filed Nov. 16, 2010, now U.S. Pat. No. 9,428,566, which is a divisional of Ser. No. 12/438,923, filed Oct. 6, 2009, now abandoned, which is a U.S. national stage of International Application No. PCT/US2007/014499, filed Jun. 20, 2007, which claims the benefit under 35 U.S.C. § 119(e) from U.S. Application No. 60/815,491, filed Jun. 20, 2006. The contents of the foregoing applications are hereby incorporated by reference in their entirety.

### REFERENCE TO AN ELECTRONIC SEQUENCE LISTING

The contents of the electronic sequence listing (244713.xml; Size: 363,342 bytes; and Date of Creation: Apr. 2, 2025) is herein incorporated by reference in its entirety.

### BACKGROUND OF THE INVENTION

This invention relates to peptides having antibacterial and antifungal properties. The invention also concerns the preparation of these peptides and compositions containing the same which may be used in agriculture and for human or animal therapy.

Nature provides a context in which organisms across the phylogenetic spectrum are confronted by potential microbial pathogens. In turn, natural selection provides a corresponding requirement for rapid and effective molecular stratagems of host defense against unfavorable microbial infection. Antimicrobial peptides represent a key result of this co-evolutionary relationship. While higher organisms have evolved complex and adaptive immune systems, virtually all organisms rely upon primary innate immune mechanisms that are rapidly deployed to ward off microbial invasion. Discoveries over the last decade indicate that antimicrobial peptides elaborated by essentially all organisms play integral roles in these innate mechanisms of antimicrobial host defense.

Antimicrobial peptides may be generally categorized as those with or without disulfide bridges. Those that contain disulfides commonly adopt β-sheet structures, while those lacking cysteine crosslinkages often exhibit α-helical conformation. Antimicrobial peptides from both classes have a number of conserved features that likely contribute to their toxicity to microorganisms, including: 1) small size, typically ranging from 12-50 amino acids; 2) cationicity, with net charges ranging from +2 to +7 at pH 7; and 3) amphipathic stereogeometry conferring relatively polarized hydrophilic and hydrophobic facets (Yeaman and Yount, Pharmacol. Rev. 55:27 (2003)). The limited size of these polypeptides places restrictions on the structural repertoire available to meet these requirements. Despite these limitations, as a group antimicrobial peptides display a high degree of variability at non-conserved sites, with amino acid substitution rates on the order of those associated with positive selection (A. L. Hughes, Cell. Mol. Life Sci. 56:94 (1999)). These observations are consistent with the hypothesis that co-evolutionary selective pressures drive host-pathogen interactions (M. J. Blaser, N. Engl. J. Med. 346:2083 (2002)).

Amino acid sequence motifs have previously been identified within certain antimicrobial peptide subclasses (eg., the cysteine array in certain mammalian defensins; White et al., Curr. Opin. Struct. Biol. 5:521 (1995)). Yet, comparatively little is known about more comprehensive relationships uniting all antimicrobial peptides. Conventional sequence analyses performed have yielded limited sequence conservation, and no universal structural homology has been identified amongst antimicrobial peptides. If present, such a consensus motif across the diverse families of antimicrobial peptides would provide insights into the mechanism of action of these molecules, yield information on the evolutionary origin of these sequences, and allow prediction of antimicrobial activity in molecules recognized to have other functions.

The ability of certain bacteria such as M. tuberculosis and S. aureus among others, to develop resistance to antibiotics represents a major challenge in the treatment of infectious disease. Unfortunately, relatively few new antibiotic drugs have reached the market in recent years. Methods for administering new classes of antibiotics might provide a new scientific weapon in the war against bacterial infections.

There are only a handful of antifungal drugs known for the treatment of mammals. In fact, there were only ten FDA approved antifungal drugs available in 2000 for the treatment of systemic fungal infections. There are three important classes of fungal drugs for the treatment of systemic infections: polyenes, pyrimidines, and azoles. The FDA has also approved certain drugs belonging to other classes for topical treatment of fungal infections. Certain traditional antifungal drugs may have a significant toxicity, and certain antifungal drugs available for use in treatment have a limited spectrum of activity. Still further, certain antifungal drugs among the azoles can have interactions with coadministered drugs, which can result in adverse clinical consequences. As with the antibiotics, certain fungi have developed resistance to specific antifungal drugs. Patients with compromised immune systems (e.g., AIDS) patients have in some cases had prolonged exposure to fluconazole for both prophylactic and therapeutic purposes. In 2000, increased use of the drug fluconazole correlated with the isolation of increasing numbers of resistant infectious fungi among AIDS patients. Methods of using a new class of antifungal drugs could make new treatments for fungal infections possible.

Invasive mycoses are very serious infections caused by fungi found in nature and which become pathogenic in immunocompromised persons. Immunosuppression may be the result of various causes: corticotherapy, chemotherapy, transplants, HIV infection. Opportunistic fungal infections currently account for a high mortality rate in man. They may be caused by yeasts, mainly of Candida type, or filamentous fungi, chiefly of Aspergillus type. In immunosuppressed patients, failure of antifungal treatment is frequently observed on account of its toxicity, for example, treatment with Amphotericin B, or the onset of resistant fungi, for example resistance of Candida albicans to nitrogen derivatives. It is, therefore, vital to develop new antifungal medicinal products derived from innovative molecules. In this context, antimicrobial peptides offer an attractive alternative.

Antimicrobial peptides are ubiquitous in nature and play an important role in the innate immune system of many species. Antimicrobial peptides are diverse in structure, function, and specificity. A number of antimicrobial peptides occur naturally as “host-defense” compounds in humans, other mammals, amphibians, plants and insects, as well as in bacteria themselves. Synthetic antimicrobial peptides have also been described, including highly amphipathic peptides whose amino acid sequences are related to or derived from the sequences of various viral membrane proteins.

The significant advantage of peptide antimicrobials resides in the global mechanism of their anti-microbial action; because peptides have an inherent capacity to bind and penetrate biological membranes, these compounds act by physically disrupting cellular membranes, usually causing membrane lysis and eventually cell death. Organisms such as bacteria have little ability to combat this physical mechanism and acquire resistance.

Thus, there exists a need for employing multidimensional proteomic techniques to determine structural commonalities amongst peptides elaborated in phylogenetically diverse organisms—microbial to human—and explore the potential convergence of structural paradigms in these molecules. The present invention satisfies this need and provides related advantages as well.

### SUMMARY OF THE INVENTION

The present invention provides novel kinocidin peptides comprising a C-terminal portion of a kinocidin, wherein the C-terminal portion encompasses an α-helical secondary structure and further displays antimicrobial activity. The kinocidin peptides of the invention are derived from and correspond to a C-terminal portion of a kinocidin, wherein the kinocidin includes a γ_{KC} core and can be a CXC, CX₃C, CC, or C class chemokine. Structural, physicochemical and functional properties of this novel class of antimicrobial peptides and amino acid sequences of particular kinocidin peptides are also disclosed. The invention also provides related antimicrobial methods.

### BRIEF DESCRIPTION OF THE DRAWINGS

FIG. 1 shows conventional antimicrobial peptide structure classification and distribution. Relationship amongst structure and predominance is summarized for the commonly recognized antimicrobial peptide classes. Concatenation represents the proportionate distribution of peptides encompassing a given structural class, as calculated from the Antimicrobial Sequences Database. Numbers of peptides classified in each group are indicated in brackets for each class.

FIG. 2A shows multiple sequence alignment of antimicrobial peptides examined. The MSA of the β-sheet peptide study set was generated using the Clustal W tool (Version 1.81; Higgins and Sharp, Gene 73:237 (1988); Higgins and Sharp, Comput. Appl. Biosci. 5:151 (1989)), as visualized with Jalview (M. Clamp, Jalview—java multiple alignment editor, version 1.7b (1998). Public domain (www.ebi.ac.uk/jalview/)). The coloration scheme is formatted to the Clustal degree of conservation. Individual peptides are designated by the following information series: peptide name, (source genus), and [Swiss Protein accession code]: protegrin 1, (Sus), [3212589] (SEQ ID NO: 43); gomesin, (Acanthoscurria), [20664097] (SEQ ID NO: 44); drosomycin, (Drosophila), [2780893] (SEQ ID NO: 45); MGD-1, (Mytilus), [12084380] (SEQ ID NO: 46); tachyplesin I, (Tachypleus), (SEQ ID NO: 47); mytilin A, (Mytilus), [6225740] (SEQ ID NO: 48); sapecin, (Sarcophaga), [2015208] (SEQ ID NO: 49); HNP-3, (Homo), (SEQ ID NO: 50); Ah-Amp1, (Aesculus), [6730111] (SEQ ID NO: 51); AFP-1, (Aspergillus), [1421258] (SEQ ID NO: 52); mBD-8, (Mus), [15826276] (SEQ ID NO: 53); thanatin, (Podisus), [6730068] (SEQ ID NO: 54); and gaegurin-1, (Rana), [1169813] (SEQ ID NO: 55).

FIG. 2B shows convergence in the sequence patterns of cysteine-containing antimicrobial peptides. The consensus primary structural motifs were identified amongst the prototypical disulfide-containing antimicrobial peptide study set. Sequence data and disulfide arrays indicated were derived from the following sources (in descending order): α-defensins (SEQ ID NO: 56) (Yount et al., J. Biol. Chem. 274:26249 (1999)); β-defensins (SEQ ID NO: 57) (Yount et al., J. Biol. Chem. 274:26249 (1999)); insect defensins (SEQ ID NO: 58) (sapecin; Hanzawa et al., FEBS Lett. 269:413 (1990)); insect CS-αβ peptides (SEQ ID NO: 59) (drosomycin; Landon et al., Protein Sci. 6:1878 (1997)); plant CS-αβ peptides (SEQ ID NO: 60) (Ah-AMP-1; Fant et al., Proteins 37:388 (1999)); crustacea CS-αβ peptides (SEQ ID NO: 61) (MGD-1; Yang et al., Biochemistry 39:14436 (2000)); gaegurin (SEQ ID NO: 64) (gaegurin-1; Park et al., Biochem. Biophys. Res. Commun. 205:948 (1994)); protegrin (SEQ ID NO: 62) (protegrin-1; Fahrner et al., Chem. Biol. 3:543 (1996)); gomesin (SEQ ID NO: 65) (Silva et al., J. Biol. Chem. 275:33464 (2000); Mandard et al., Eur. J. Biochem. 269:1190 (2002)); thanatin (SEQ ID NO: 66) (Mandard et al., Eur. J. Biochem. 256:404 (1998)); tachyplesin (SEQ ID NO: 67) (tachyplesin I; Nakamura et al., J. Biol. Chem. 263:16709 (1988)); mytilin (SEQ ID NO: 63) (mytilin Charlet et al., J. Biol. Chem. 271:21808 (1996)); AFP-1 (SEQ ID NO: 68) (Campos-Olivas et al., Biochemistry 34:3009 (1995)). The primary sequences corresponding to the γ-core motif are outlined in red (see FIG. 4). Sequences are shown in their conventional dextromeric orientations (N- to C-termini from left to right) unless indicated to be projected in a levomeric orientation (levo; C- to N-termini from left to right).

FIG. 3A-H shows conservation of 3-dimensional signatures amongst antimicrobial peptides. Three-dimensional structural alignments were carried out by the combinatorial extension method (Shindyalov and. Bourne, Protein Eng. 11:739 (1998)), visualized using Protein Explorer (Martz, Trends Biochem. Sci. 27:107 (2002)). Comparisons are between (Ah-AMP-1 ([1BK8], Aesculus, horsechestnut tree) and (peptide name, [PDB accession code], genus, common name; RMSD): protegrin-1 ([1PG1], Sus, domestic pig; RMSD 1.2 Å; panels A and B); drosomycin ([1MYN], Drosophila, fruit fly; RMSD 1.4 Å; panels C and D); HNP-3 ([1DFN]; Homo, human; RMSD 3.2 Å; panels E and F); and magainin-2 ([2MAG]; Xenopus, frog; Gesell et al., J. Biomol. NMR 9:127 (1997); RMSD 2.6 Å; panels G and H). Respective amino-and carboxy-termini are indicated in panels A, C, E and G. Panels A, C, E, and G use the Clustal degree of 2° structure conservation coloration scheme. Panels B, D, F, and H employ the DRUMS polarity-2 color scheme, in which hydrophobic residues are colored gray, while hydrophilic residues are colored purple. By convention, cysteine residues are indicated as hydrophilic, although in these peptides, they are oxidized (cystine) and colored gray indicating hydrophobicity. Amino-(N-) and carboxy-(C-) termini for comparative peptides are denoted as N1 or N2 and C1 or C2, respective of peptides designated 1 or 2. Relative positions of the disulfide bonds are indicated as dotted yellow lines in panels A-H. See Table II for additional references. Proteins were visualized using Protein Explorer as described by Martz, Trends Biochem. Sci. 27, 107-109 (2002).

FIG. 3I-L demonstrates the absence of the γ-core signature in non-antimicrobial peptides. Three-dimensional conformity between prototypic antimicrobial and non-antimicrobial peptides was determined as described in FIG. 3A-H. Representative comparisons are between the antimicrobial peptide Ah-AMP1, and the following non-antimicrobial peptides (identified as formatted in FIG. 3A-H): allergen-5 ([2BBG], Ambrosia, ragweed; RMSD 6.5 Å; panel I); metallothionein II ([1AOO], Saccharomyces, yeast; RMSD 5.3 Å; panel J); TGF-α ([3TGF], Homo, human; RMSD 4.7 Å; panel K); and ferredoxin ([2FDN], Clostridium, bacterium; RMSD 7.4 Å; panel L). Each non-antimicrobial comparator peptide (blue) is shown in maximal alignment with Ah-AMP1 (gray). Amino-(N) and carboxy-(C) termini are indicated as defined in FIG. 3A-H. See Table II for references.

FIG. 4 shows conservation of the γ-core motif amongst disulfide-containing antimicrobial peptides. The conserved γ-core motif (red) is indicated with corresponding sequences (GXC or CXG-C motifs are denoted in red text). Examples are organized into four structural groups relative to the γ-core. Group (γ): protegrin-1, [1PG1] (SEQ ID NO: 69); gomesin [1KFP] (SEQ ID NO: 70); tachyplesin-1 [1MA2] (SEQ ID NO: 71); RTD-1 [1HVZ] (SEQ ID NO: 72); thanatin [8TFV] (SEQ ID NO: 73); hepcidin [1M4F]) (SEQ ID NO: 74); Group (γ-α): sapecin [ILV4] (SEQ ID NO: 75); insect defensin A [1ICA] (SEQ ID NO: 76); heliomicin [1I2U] (SEQ ID NO: 77); drosomycin [1MYN] (SEQ ID NO: 78); MGD-1 [IFJN] (SEQ ID NO: 79); charybdotoxin [2CRD] (SEQ ID NO: 80); Group (β-γ): HNP-3 [1DFN] (SEQ ID NO: 81); RK-1 [1EWS] (SEQ ID NO: 82); BNBD-12 [1BNB] (SEQ ID NO: 83); HBD-1 [1E4S] (SEQ ID NO: 84); HBD-2 [1E4Q] (SEQ ID NO: 85); mBD-8 [1E4R] (SEQ ID NO: 86)); and Group (β-γ-α): Ah-AMP-1 [1BK8] (SEQ ID NO: 87); Rs-AFP-1 [1AYJ] (SEQ ID NO: 88); Ps-Def-1 [IJKZ] (SEQ ID NO: 89); γ-1-H-thionin [1GPT] (SEQ ID NO: 90); γ-1-P-thionin [1GPS] (SEQ ID NO: 91); and brazzein [1BRZ] (SEQ ID NO: 92). Protegrin, gomesin, tachyplesin, RTD-1, and thanatin γ-core sequences (Group y) are depicted in levomeric orientation. Other peptide data are formatted as in FIG. 3. See Table II for additional references.

FIG. 5A-C shows iterations of the 3-dimensional γ-core motif. Amino acid consensus patterns of the three γ-core sequence isoforms are shown. Coloration represents the most common residue (>50% frequency) at a given position, as adapted from the RASMOL schema: cysteine (C), yellow; glycine (G), orange; lysine or arginine, royal blue; serine or threonine, peach; leucine, isoleucine, alanine or valine, dark green; aromatic, aqua; and variable positions (<50% consensus), gray.

FIG. 6A-I shows molecules exemplifying structure-based or activity-based validation of the multidimensional signature model. Representative molecules retrieved using the enantiomeric sequence patterns were identified (Table III) and analyzed for presence or absence of a γ-core motif as described. Thus, appropriate molecules were identified to challenge each of the respective model-based predictions. Three-dimensional structures visualized using Protein Explorer are indicated for: brazzein ([1BRZ], Pentadiplandra, J'Oblie berry, panels A, D, and G; Caldwell et al., Nat. Struct. Biol. 5:427 (1998)); charybdotoxin ([2CRD], Leiurus, scorpion, panels B, E, and H; Bontems et al,. Biochemistry 31:7756 (1992)), tachyplesin I ([1MA2], Tachypleus, horseshoe crab, panels C, F, and I); and metallothionein II (see FIG. 3). As in FIG. 3, comparative panels A-C use the Clustal degree of 2° structure conservation coloration scheme. Panels D-F employ the DRUMS polarity-2 color scheme, in which hydrophobic residues are colored gray, and hydrophilic residues are colored purple. As in FIG. 4, amino acids comprising the γ-core motifs are highlighted in red (panels G-I) within the 3-dimensional structures of these representative peptides. Other data are formatted as in FIG. 3.

FIG. 7A-H shows experimental validation of the predictive accuracy of the multidimensional signature model. Standard radial diffusion assays were conducted using 10 μg of specified peptide: defensin HNP-1 (HNP); brazzein (BRZ); charybdotoxin (CTX); or metallothionein II (MTL). Recombinant brazzein reflecting the published 3-dimensional structure (1BRZ) as determined by nuclear magnetic resonance spectroscopy was kindly provided by Drs. J. L. Markley and F. M. Assadi-Porter, the University of Wisconsin 25. Charybdotoxin, metallothionein II, and defensin HNP-1 were obtained from commercial sources. Antimicrobial activity was assessed using a well-established solid-phase diffusion method as described by Tang et al., Infect. Immun. 70:6524-6533 (2002). Assays included well characterized organisms: Staphylococcus aureus (ATCC 27217, Gram-positive coccus); Bacillus subtilis (ATCC 6633, Gram-positive bacillus); Escherichia coli (strain ML-35, Gram-negative bacillus); and Candida albicans (ATCC 36082, fungus). In brief, organisms were cultured to logarithmic phase and inoculated (10⁶ colony forming units/ml) into buffered molecular-biology grade agarose at the indicated pH. Peptides resuspended in sterile deionized water were introduced into wells formed in the underlay, and incubated for 3 h at 37° C. Nutrient-containing overlay medium was then applied, and assays incubated at 37° C. or 30° C. for bacteria or fungi, respectively. After 24 h, zones of complete or partial inhibition were measured. All assays were repeated independently a minimum of two times at pH 5.5 (panels A-D) or pH 7.5 (panels E-H) to assess the influences of pH on peptide antimicrobial activities versus microorganisms. Histograms express mean (±standard deviation) zones of complete (blue) or incomplete (yellow) inhibition of growth. These data establish the direct antimicrobial activities of brazzein and charybdotoxin. Metallothionein II lacked antimicrobial activity under any condition assayed. Note differences in scale.

FIG. 8A shows phylogenetic relationship amongst structural signatures in prototypical antimicrobial peptides. Relative evolutionary distances are indicated at branch nodes in this average distance dendrogram (Saito and Nei, Mol. Biol. Evol. 4:406 (1987)). Representative peptides for which structures have been determined are (descending order): AFP (AFP-1; Aspergillus, fungal); PRG1 (Protegrin-1; Sus, domestic pig); GOME (Gomesin; Acanthoscurria, spider); THAN (Thanatin; Podisus, soldier bug); HNP3 (Human neutrophil peptide-3; Homo, human); MGDI (MGD-1; Mytilus, mussel); SAPE (Sapecin; Sarcophaga, flesh fly); MBD8 (Murine β-defensin-8; Mus, mouse); DMYN (Drosomycin; Drosophila, fruit fly); Ah-AMP1 (AMP-1; Aesculus, horsechestnut tree). Color schema are the Clustal degree of 2° structure conservation. These data illustrate the concept that the γ-core is the common structural element in these peptides, suggesting it is an archetype motif of the antimicrobial peptide signature (see FIG. 4).

FIG. 8B shows modular iterations of multidimensional signatures in disulfide-stabilized antimicrobial peptides. Distinct configurations integrating the γ-core are found in naturally occurring antimicrobial peptides from diverse organisms. Specific examples are used to illustrate this theme (modular formulae are as described in the text): [γ], Protegrin-1; [γα₁], MGD-1; [γβ₁], HNP-3; and [γα₁β₁], Ah-AMP-1. Color schema and peptide identification are as indicated in FIG. 3 (A, C, E, G).

FIG. 9 shows conservation of the multidimensional signature in disulfide-containing antimicrobial peptides. This triple alignment demonstrates the dramatic 3-dimensional conservation in antimicrobial peptides from phylogenetically diverse species spanning 2.6 billion years of evolution: fruit fly (Drosophila; [1MYN]), mussel (Mytilus; [1FJN]) and horsechestnut tree (Aesculus; [1BK8]). The striking degree of 3-dimensional preservation reflects a unifying structural code amongst these broad classes of disulfide containing host defense effector molecules. Alignment was carried out using the Vector Alignment Search Tool (VAST) available through the National Center for Biotechnology Information (NCBI). Secondary structure is indicated by the CN3D coloration schema: sheet, gold; helix, green; turn/extended, blue.

FIG. 10A-E, depict amino acid sequences of γ-core signature motifs amongst disulfide-containing antimicrobial peptides. Nomenclature and coloration are as indicated in FIGS. 2 and 3 of the primary manuscript; standard abbreviations are used for peptide names where appropriate. Lavender shading of molecule identities in Groups IID and IIIB indicates peptides aligned in the levomeric orientation. These sequences correspond to the γ-core pattern map as depicted in FIG. 3 of the primary manuscript. FIG. 10A depicts amino acid sequences of SEQ ID NOS: 93-132, in order from top to bottom. FIG. 10B depicts amino acid sequences of SEQ ID NOS: 133-173, in order from top to bottom. FIG. 10C depicts amino acid sequences of SEQ ID NOS: 173-210, in order from top to bottom. FIG. 10D depicts amino acid sequences of SEQ ID NOS: 211-238, in order from top to bottom. FIG. 10E depicts amino acid sequences of SEQ ID NOS: 239-255, in order from top to bottom.

FIG. 11A-B show peptides with predicted antimicrobial activity based on the multidimensional signature. Candidate peptides were identified by VAST alignment, 3D-RMSD, and manual comparisons; all RMSD scores compared with Ah-AMP-1 (1BK8; Aesculus); threshold typically >4.5 excluded; each sequence is identified by NCBI accession number. FIG. 11A depicts amino acid sequences of SEQ ID NOS: 256-390, in order from top to bottom. FIG. 11 B depicts amino acid sequences of SEQ ID NOS: 291-315, in order from top to bottom.

FIG. 12 shows alignment of C, CC and CXC class human chemokines (SEQ ID NOS: 316-351). The highlighted GX₃C motif [glycine (G), orange; cysteine (C), yellow; proline (P), aqua] corresponds to the γ_{KC} core signature (outlined in red). Conserved cysteine residues beyond the γ_{KC} core are shaded gray. Gaps were introduced to achieve maximal alignment; * indicates truncated sequence.

FIG. 13 demonstrates conservation of the γ-core domain within kinocidins (γ_{KC} core). Recurring iterations of the γ_{KC} core motif (red) are indicated with corresponding sequences (GX₃C) denoted in red or gold text. A comparator antimicrobial peptide (Ah-AMP-1) is also shown to illustrate structural similarities between the γ_{KC} motif and that present in antimicrobial peptides (γ_{AP}). Proteins were visualized using protein explorer (Martz, E. (2002) Trends Biochem Sci 27, 107-9.). Amino acid sequences of SEQ ID NOS: 352-358 are depicted.

FIG. 14 shows solid-phase antimicrobial activity of human kinocidins and IL-8 subdomains IL-8α and IL-8γ. Peptides (0.5 nmol) were introduced into wells in agarose plates buffered with MES (2.0 mM, pH 5.5) or PIPES (10.0 mM, pH 7.5). Antimicrobial activity was assessed as the zone of complete (blue) or partial (red) inhibition around the well. Abbreviations are: Native IL-8 (IL-8); IL-8α, (α); IL-8γ, (γ); IL-8α+IL-8γ (α+γ); RANTES, (RAN); GRO-α, (GRO); MCP-1, (MCP); lymphotactin, (LYM); platelet factor-4, (PF-4); and HNP-1, (HNP). Histograms are means±SEM (minimum n=2).

FIG. 15 shows solution-phase microbicidal activity of native IL-8 and subdomains IL-8α and IL-8-γ. One million CFU of the indicated microorganism per milliliter were incubated with peptide (0.00125-20.0 nmol/ml) in either MES (2.0 mM, pH 5.5) or PIPES (10.0 mM, pH 7.5) for one hour at 37° C. Surviving CFU were enumerated and are described as change in the initial log10 CFU. ○, S. aureus ATCC 27217; •, S. typhimurium strain 5990 s; □, C. albicans ATCC 36082. Data are means±SD (minimum n=2).

FIG. 16A-B show spectroscopy for IL-8 structural domains. Spectra were determined for the IL-8γ and IL-8α peptides (0.1 mM) in sodium phosphate (10.0 mM, pH 5.5) or PIPES (10.0 mM, pH 7.5) buffer. [. . . ], (IL-8α); [______], (IL-8-γ_{KC}).

FIG. 17A-B show computational modeling of IL-8 structural domains. Three-dimensional models of IL-8α (A) and IL-8γ (B) peptides were created using homology and energy-based methods. Model peptide alpha-carbon backbones were visualized using PyMOL (version 0.97; 2004).

FIG. 18A-B show antimicrobial efficacy of IL-8_{α} in human blood and blood-derived matrices as compared with artificial media (MHB) at pH 5.5 and 7.2. Panel 18 (A) shows co-incubation of IL-8_{α} and the organism simultaneously added to the test biomatrix or medium; Panel 18 (B) shows pre-incubation of IL-8_{α} in biomatrices or media for 2 h at 37° C. prior to introduction of the organism. The E. coli inocula (INOC) were 10⁵ CFU/ml, and the threshold of sensitivity was considered 0.3 log10 CFU/ml.

### DETAILED DESCRIPTION OF THE INVENTION

This application file contains drawings executed in color. Copies of this patent or application publication with color drawings will be provided by the Office upon request and payment of the necessary fee.

This invention provides antimicrobial kinocidin peptides and related methods of use. The antimicrobial kinocidin peptides of the invention encompass at least a portion of the C-terminal α-helical region of a kinocidin, wherein the C-terminal portion encompasses an α-helical secondary structure. The kinocidin peptides of the invention are derived from and correspond to a C-terminal portion of a kinocidin that includes a γ_{KC} core predictive of antimicrobial activity. A kinocidin is a antimicrobial CXC, CX₃C, CC, or C class chemokine.

The kinocidin peptide can include up to the entire C-terminal α-helix of the corresponding kinocidin from which it is derived. A kinocidin peptide generally has physicochemical properties within the ranges set forth in Table IV below and also has antimicrobial activity

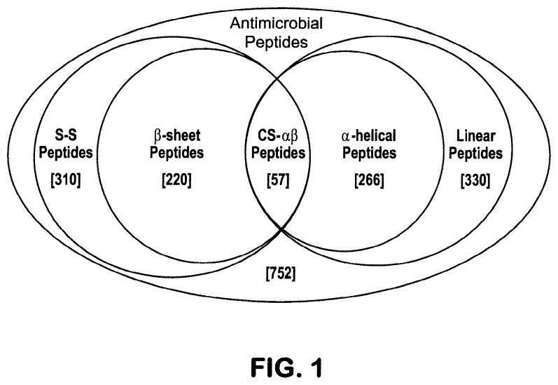

The term “kinocidin,” as used herein refers to a chemokine having microbicidal activity. As described herein, the ability of a chemokine to exert antimicrobial activity can be predicted based on the presence of the γ_{KC} core consensus formula, and specific physicochemical patterns of amphipathicity, charge distribution, and proline positioning within the chemokine (see FIG. 1). More than 40 human chemokines have been characterized and are classified into four groups according to conserved N-terminal cysteine motifs: CXC (α-chemokines), CC (β-chemokines), C, and CX₃C (Hoffmann et al. (2002) J Leukoc Biol 72, 847-855).

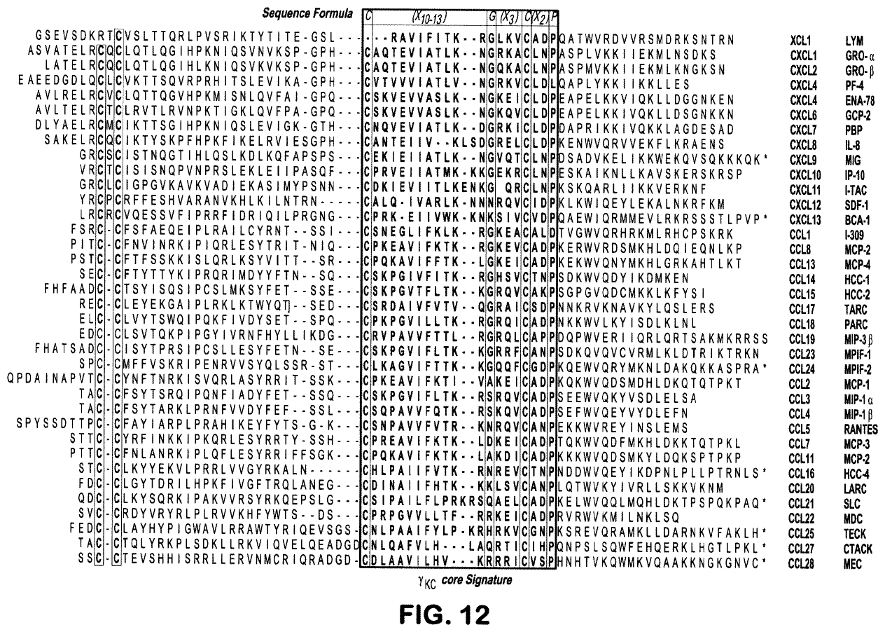

As used herein, the term “kinocidin peptide” refers to a peptide that has microbicidal activity and that contains all or a portion of a C-terminal α-helix of a kinocidin. In structural terms, a kinocidin peptide of the invention is characterized by corresponding to a C-terminal portion of a kinocidin. As described throughout this disclosure, a kinocidin can be selected based on the presence of the γ_{KC} core consensus formula, and specific physicochemical patterns of amphipathicity, charge distribution, and proline positioning within the chemokine (see FIG. 12). In functional terms, a kinocidin peptide has antimicrobial, for example, antimicrobial and/or antifungal activity, which can be confirmed via routine methods described in the art and exemplified herein. A kinocidin peptide of the invention can have any length provided the requisite activity is present, for example, can be between 50 or less, 45 or less, 40 or less, 35 or less, 34 or less, 33 or less, 32 or less, 30 or less, 29 or less, 28 or less, 27 or less, 26 or less, 25 or less, 24 or less. 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, and preferably between 10 and 40, more preferably between 12 and 38, more preferably between 14 and 32 amino acids in length. Also encompassed within the term are dimers and other multimers, truncated molecules, and molecules that contain repetitions of particular subsequences within the motif as long as the peptide has microbicidal activity and that contains all or a portion of a C-terminal α-helix of a kinocidin.

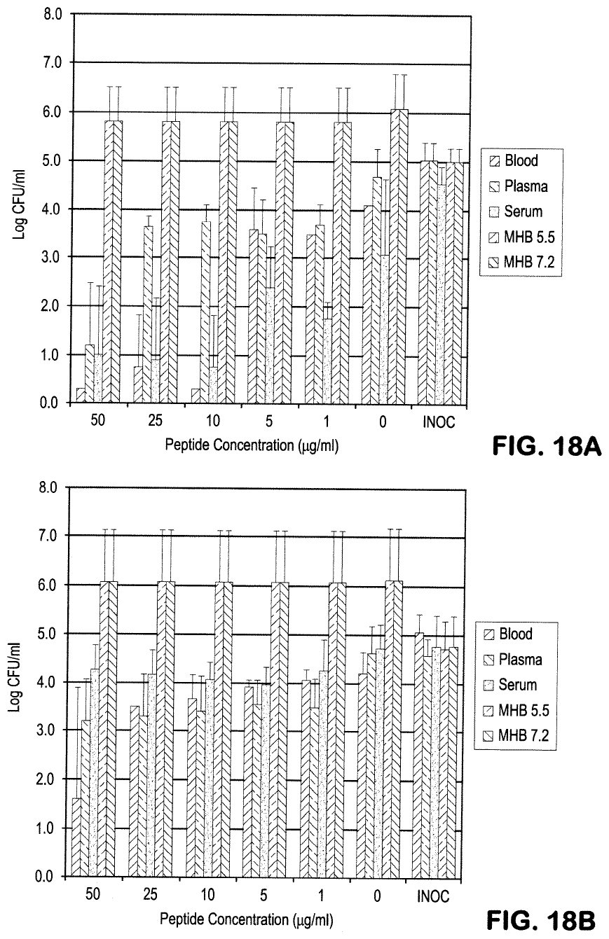

The invention provides particular kinocidin peptides, each comprising a C-terminal portion of a kinocidin having an α-helical secondary structure and antimicrobial activity, and further having a γ_{KC} core. As described herein, the kinocidin can be a CXC, CC, or C class chemokine. In one embodiment, the invention provides a kinocidin peptide that corresponds to a CXC chemokine has the amino acid sequence KENWVQRVVEKFLKRAENS (SEQ ID NO: 1). In another embodiment, the invention provides a kinocidin peptide that corresponds to a CXC chemokine has the amino acid sequence QAPLYKKIIKKLLES (SEQ ID NO: 2). In a further embodiment, the invention provides a kinocidin peptide that corresponds to a CXC chemokine has the amino acid sequence ASPIVKKIIEKMLNSDKSN (SEQ ID NO: 3). In a further embodiment, the invention provides a kinocidin peptide that corresponds to a CXC chemokine has the amino acid sequence DAPRIKKIVQKKLAGDES (SEQ ID NO: 4). Additional kinocidin peptides of the invention based on Human CXC, CC and C Chemokine C-terminal α-Helical Domains are set forth in FIG. 18.

TABLE 1
Antimicrobial peptides of the invention based on Human CXC, CC and C
Chemokine C-terminal α-Helical Domains.
Group 1
Based on Human CXC Chemokine α-Helical Domains

| Origin | Name | Amino Acid Sequence (SEQ ID NO:) |
|---|---|---|
| CXCL1/GRO-alpha | Aegicidin hGro-α-C1 | ASPIVKKIIEKMLNSDKSN (3) |
| CXCL2/MIP2-alpha | Aegicidin hMIP2-α-C1 | ASPMVKKIIEKMLKNGKSN (5) |
| CXCL3/GRO-beta | Aegicidin hGro-β-C1 | ASPMVQKIIEKILNKGSTN (6) |
| CXCL4/PF-4 | | QAPLYKKIIKKLLES (2) |
| CXCL5/ENA-78 | Aegicidin hENA-78-C1 | EAPFLKKVIQKILDGGNKEN (7) |
| CXCL6/GCP-2 | Aegicidin hGCP-2-C1 | EAPFLKKVIQKILDSGNKKN (8) |
| CXCL7/PBP, | | DAPRIKKIVQKKLAGDESAD (9) |
| CTAP3, NAP2 | | |
| CXCL8/IL-8 | Aegicidin HIL-8-C1 | KENWVQRVVEKFLKRAENS (1) |
| CXCL9/MIG | Aegicidin hMIG-C1 | DSADVKELIKKWEKQVSQKKKQKNGKK (359) |
| CXCL10/IP-10 | Aegicidin hIP-10-C1 | ESKAIKNLLKAVSKERSKRSP (10) |
| CXCL11/I-TAC | Aegicidin hI-TAC-C1 | KSKQARLIIKKVERKNF (11) |
| CXCL12/SDF-1 | Aegicidin hSDF-1-C1 | KLKWIQEYLEKALNKRFKM (12) |
| CXCL13/BCA-1 | Aegicidin hBCA-1-C1 | QAEWIQRMMEVLRKRSSSTLPVPVFKRKIP* (13) |
| CXCL14/BRAK | Aegicidin hBRAK-C1 | KLQSTKRFIKWYNAWNEKRRVYEE (14) |

Group 2
Based on Human CC Chemokine α-Helical Domains

| Origin | Name | Amino Acid Sequence (SEQ ID NO:) |
|---|---|---|
| CCL1/I-309 | Aegicidin hI-309-C1 | TVGWVQRHRKMLRHCPSKRK (15) |
| CCL2/MCP-1 | Aegicidin hMCP-1-C1 | KQKWVQDSMDHLDKQTQTPKT (16) |
| CCL3/MIP-1alpha | Aegicidin hMIP-lα-C1 | SEEWVQKYVSDLELSA (17) |
| CCL4/MIP-1beta | Aegicidin hMIP-1β-C1 | SESWVQEYVYDLELN (18) |
| CCL5/RANTES | Aegicidin hRANTES-C1 | EKKWVREYINSLEMS (19) |
| CCL7/MCP-3 | Yeaman & Yount | TQKWVQDFMKHLDKKTQTPKL (20) |
| | terminology: Aegicidin | |
| | hMCP3-C1 | |
| CCL8/MCP-2 | Aegicidin hMCP-2-C1 | KERWVRDSMKHLDQIFQNLKP (21) |
| CCL11/EOTAXIN | Aegicidin hEOTx-C1 | KKKWVQDSMKYLDQKSPTPKP (22) |
| CCL13/MCP-4 | Aegicidin hMCP-4-C1 | KEKWVQNYMKHLGRKAHTLKT (23) |
| CCL14/HCC-1 | Aegicidin hHCC-1-C1 | SDKWVQDYIKDMKEN (24) |
| CCL15/HCC-2 | Aegicidin hHCC-2-C1 | SGPGVQDCMKKLKPYSI (25) |
| CCL16/HCC-4 | Aegicidin hHCC-4-C1 | NDDWVQEYIKDPNLPLLPTRNLSTVKII (26) |
| CCL17/TARC | Aegicidin hTARC-C1 | NNKRVKNAVKYLQSLERS (27) |
| CCL18/PARC | Aegicidin hPARC-C1 | NKKWVQKYISDLKLNA (28) |
| CCL19/MIP-3beta | Aegicidin hMIP-3β-C1 | DQPWVERIIQRLQRTSAKMKRRSS (29) |
| CCL20/LARC | Aegicidin hLARC-C1 | KQTWVKYIVRLLSKKVKNM (30) |
| CCL21/SLC | Aegicidin hSLC-C1 | KELWVQQLMQHLDKTPSPQKPAQG (31) |
| CCL22/MDC | Aegicidin hMDC-C1 | RVPWVKMILNKLSQ (32) |
| CCL23/MPIF-1 | Aegicidin hMPIF-1-C1 | SDKQVQVCVRMLKLDTRIKTRKN (33) |
| CCL24/MPIF-2 | Aegicidin hMPIF-2-C1 | KQEWVQRYMKNLDAKQKKASPRAR (34) |
| CCL25/TECK | Aegicidin hTECK-C1 | KSREVQRAMKLLDARNK* (35) |
| CCL27/SKINKINE | Aegicidin hSkine-C1 | QNPSLSQWFEHQERKLHGTLPKLNFGMLRKMG (36) |
| CCL28/CCK1 | Aegicidin hCCK-1-C1 | HNHTVKQWMKVQAAKKNGKGN* (37) |

Group 3
Peptides Based on C Chemokine α-Helical Domains

| Origin | Name | Amino Acid Sequence |
|---|---|---|
| CL1/Lymphotactin | Aegicidin hLym-C1 | QATWVRDVVRSMDRKSNTRNN* (38) |

Chemokines comprise a class of small secretory cytokines that play important roles in potentiating leukocyte chemonavigation and antimicrobial activity. More than 40 human chemokines have been characterized and are classified into four groups according to conserved N-terminal cysteine motifs: CXC (α-chemokines), CC (β-chemokines), C, and CX₃C (J Leukoc Biol 70, 465-466 (2001)). Chemokines have been identified in vertebrates as distant as teleost fish, and are expressed in a broad array of mammalian cell types including those of myeloid, endothelial, epithelial and fibroblast lineages (Hoffmann et al. (2002) J Leukoc Biol 72, 847-855). Of the chemokines, interleukin-8 (IL-8; or CXC-ligand 8 [CXCL8]) is perhaps the best characterized, having been first identified as neutrophil-activating factor from human monocytes more than 15 years ago (Walz et al. (1987) Biochem Biophys Res Commun 149, 755-761; Yoshimura et al. (1987) Proc Natl Acad Sci U S A 84, 9233-9237).

This invention further describes methods for identifying multidimensional protein signatures that are useful as predictors of protein activity. Prior to this invention it was unknown that proteins can be classified based on common multidimensional signatures that are predictive of activity. While exemplified herein for a subclass of antimicrobial peptides, this discovery allows for the invention methods of using experimental proteomics techniques to identify multidimensional protein signatures that are predictive of protein activity.

Based, in part, on the discovery of structural signatures in antimicrobial peptides, the invention provides methods for designing, creating or improving anti-infective agents and anti-infective strategies that are refractory to microbial resistance. The invention methods can improve the efficacy of a drug or a drug candidate by altering the multidimensional antimicrobial signature so as to approximate the multidimensional signature model.

In one embodiment, the invention provides a method for predicting antimicrobial activity of a candidate protein by determining the presence a multidimensional antimicrobial signature in a candidate protein, and comparing the multidimensional antimicrobial signature to a multidimensional antimicrobial signature model. As taught herein, the degree of similarity between the multidimensional antimicrobial signature of the candidate protein and the multidimensional antimicrobial signature model is predictive of antimicrobial activity of the candidate protein.

In a further embodiment, the invention provides a method for identifying a protein having antimicrobial activity by screening a library of candidate proteins to identify a multidimensional antimicrobial signature in a candidate protein, and subsequently comparing the multidimensional antimicrobial signature to a multidimensional antimicrobial signature model. As taught herein, the degree of similarity between the multidimensional antimicrobial signature of the candidate protein and the multidimensional antimicrobial signature model is predictive of antimicrobial activity of the candidate protein.

In a further embodiment, the invention provides a method for improving the antimicrobial activity of a protein by altering the multidimensional antimicrobial signature of the protein to increase the degree of similarity between the multidimensional antimicrobial signature of the protein and a multidimensional antimicrobial signature model. The invention also provides a protein having improved antimicrobial activity as a result of alteration of the multidimensional antimicrobial signature of the protein to increase the degree of similarity between the multidimensional antimicrobial signature of the protein and a multidimensional antimicrobial signature model.

In a further embodiment, the invention provides a method for designing a protein having antimicrobial activity by incorporating configurations that include iterations of a γ-core signature into a peptide structure that is designed. The invention also provides a protein having antimicrobial activity designed by incorporating configurations that include iterations of a γ-core signature into a peptide structure.

As used herein, the term “multidimensional protein signature” is intended to refer to a set of essential physicochemical components that make up a structural motif characteristic of a class or subclass of proteins. A multidimensional protein signature can incorporate any structural information ascertainable, including, information regarding primary structure, including amino acid sequence, composition, and distribution patterns; secondary structure, stereospecific sequence and 3-dimensional conformation. As used herein, the term “multidimensional protein signature model” refers to a protein that represents the essential structural components associated with a particular multidimensional protein signature. Individual peptides each contain an iteration of the multidimensional signature, and the essential features of this signature are reflected in the multidimensional signature model. CS-αβ family antimicrobial peptides also contain a γ_{AP} core and α-helix Kinocidins, including IL-8, share a common topology comprised of a γ_{KC} core and α-helix.

As used herein, the terms “gamma-core motif,” “γ-core,” “γ_{AP}-core,” “γ-core signature” and equivalents thereof refer to a multidimensional protein signature, in particular a multidimensional antimicrobial signature, that is characterized by two anti-parallel β-sheets interposed by a short turn region with a conserved GXC (dextromeric) or CXG (levomeric) sequence pattern integrated into one β-sheet. Additional features that characterize the γ-core motif include a hydrophobic bias toward the C-terminal aspect and cationic charge positioned at the inflection point and termini of the β-sheet domains, polarizing charge along the longitudinal axis of the γ-core.

The kinocidin γ-core (γ_{KC} core) signature is an iteration of the antimicrobial peptide γ-core (γ_{AP}), conforming to an anti-parallel β-hairpin comprised of a 13-17 amino acid pattern with a central hydrophobic region typically flanked by basic residues. The γ_{KC} core motif can be characterized by the following consensus sequence formula:

(SEQ ID NO: 39)
NH₂ [C]-[X₁₀₋₁₃]-[GX₂₋₃C]-[X₂]-[P] COOH

Human IL-8, which contains the kinocidin γ-core (γ_{KC} core) signature, has the sequence:

NH₂ CANTEIIVKLSDGRELCLDP COOH (SEQ ID NO: 40)

This fragment of the IL-8 sequence is consistent with the consensus γ_{KC}-core motif. Furthermore, many kinocidins exhibit a recurring amino acid position pattern, consistent with the consensus γ_{KC} core formula:

(SEQ ID NO: 41)
NH₂ CX₄Z₃X₀₋₂[K⁸¹]X₁₋₃G[K⁷²][B⁸⁶][Z⁹²]C[Z⁸⁶][D⁸⁶][P⁹⁵] COOH
R N

where Z represents the hydrophobic residues A, F, I, L, V, W, Y; B represents the charged or polar residues D, E, H, K, N, R, Q; C, P, or G correspond to cysteine, proline, or glycine, respectively, X indicates variable amino acid position; and numeric superscripts of bracketed positions indicate relative frequency in percent, with common alternate residues listed beneath.

As used herein, the term “protein activity” is intended to mean a functional activity or bioactivity of a protein.

Many disulfide-containing antimicrobial peptides have multiple structural domains that encompass β-sheet and/or α-helical motifs connected through an interposing region. As described herein, the invention methods provide a strategy incorporating a synthesis of proteomic and experimental methods to identify essential structural features integral to antimicrobial bioactivity that are shared amongst broad classes of antimicrobial peptides. Stereospecific sequence and 3-dimensional conformation analyses of cysteine-containing antimicrobial peptides with known structures were integrated and reduced to identify essential structural components. These approaches enabled the identification of sequence patterns and a 3-dimensional conformation integral to a multidimensional signature common to virtually all non-cyclic antimicrobial peptides containing disulfide bridges. This compelling signature transcends class-specific motifs identified previously, and reflects a unifying structural code in antimicrobial peptides from organisms separated by profound evolutionary distances.

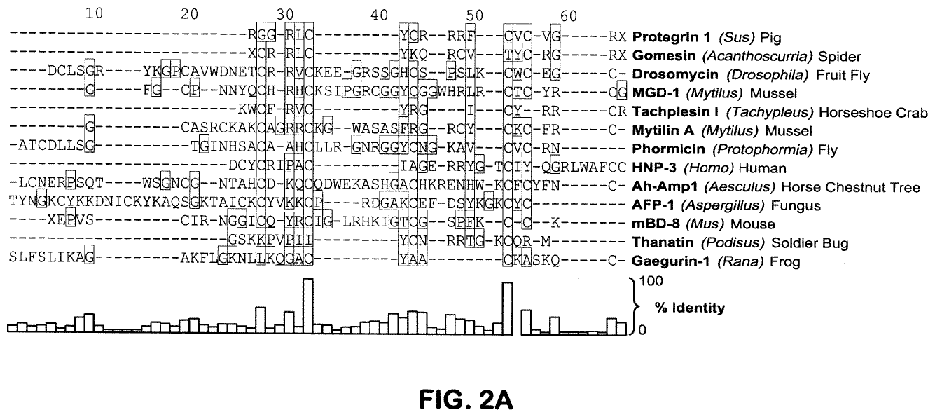

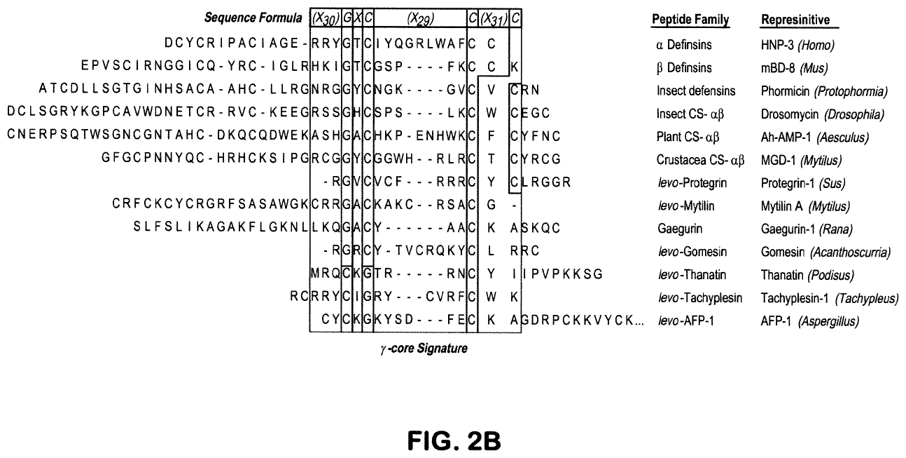

The γ-core motif is a pivotal element in the multidimensional signature of antimicrobial peptides. This motif corresponds to a hydrophobic and structurally rigid region in these molecules. Moreover, the γ-core motif consists of hallmark amino acid sequence, composition, and distribution patterns that likely facilitate antimicrobial functions. For example, patterns identified are congruent with segregation of the most polar or charged residues to solvent-accessible facets, continuity of hydrophilic or hydrophobic surfaces, and flexibility near structural extremities of these peptides. Such physicochemical properties appear to be integral to the antimicrobial mechanisms of disulfide-containing peptides such as the CS-αβ or defensin families (Yeaman and Yount, Pharmacol. Rev. 55:27 (2003); Hill et al., Science 251:1481 (1991)). Thus, the γ-core motif is more than simply a β-hairpin fold. As described herein, the γ-core component of the antimicrobial peptide signature can be derived from dextromeric or levomeric sequence patterns (FIG. 2B). The necessity for host defense against microbial pathogens has favored conservation of an effective 3-dimensional determinant, despite site-or orientation-specific variations in the primary sequences that comprise this motif. Thus, the present invention provides a method for stereospecific analysis of primary sequences that can identify structural patterns or relationships in any protein class selected by the user.

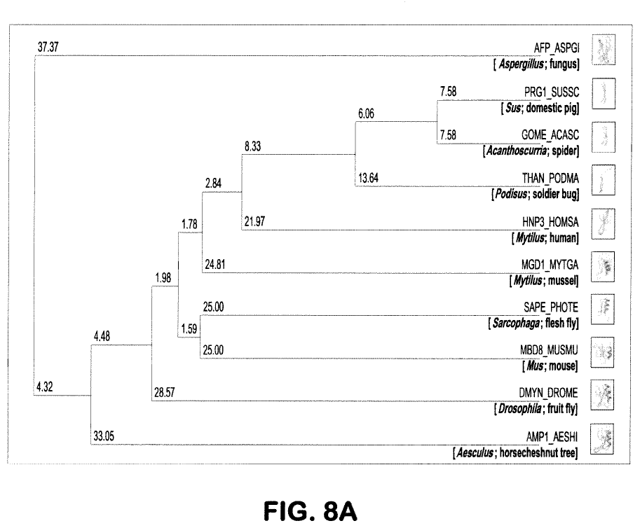

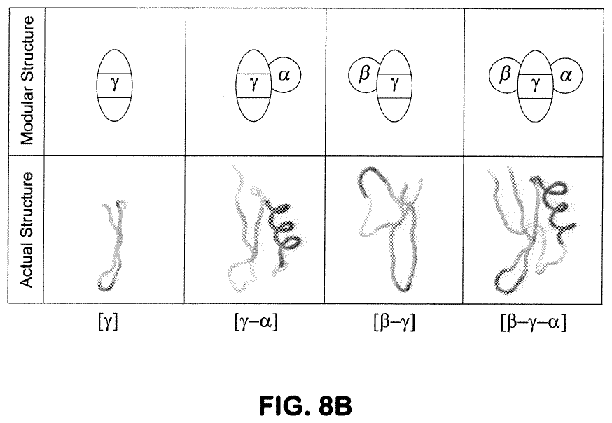

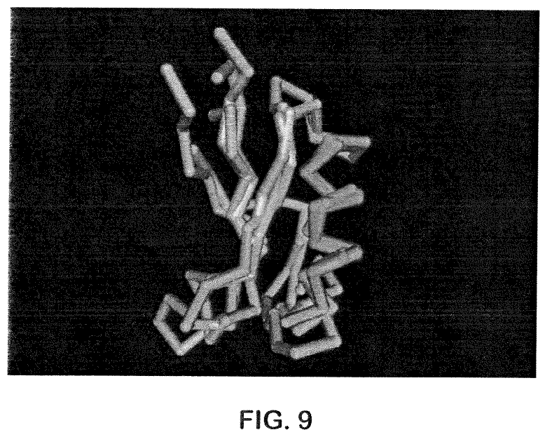

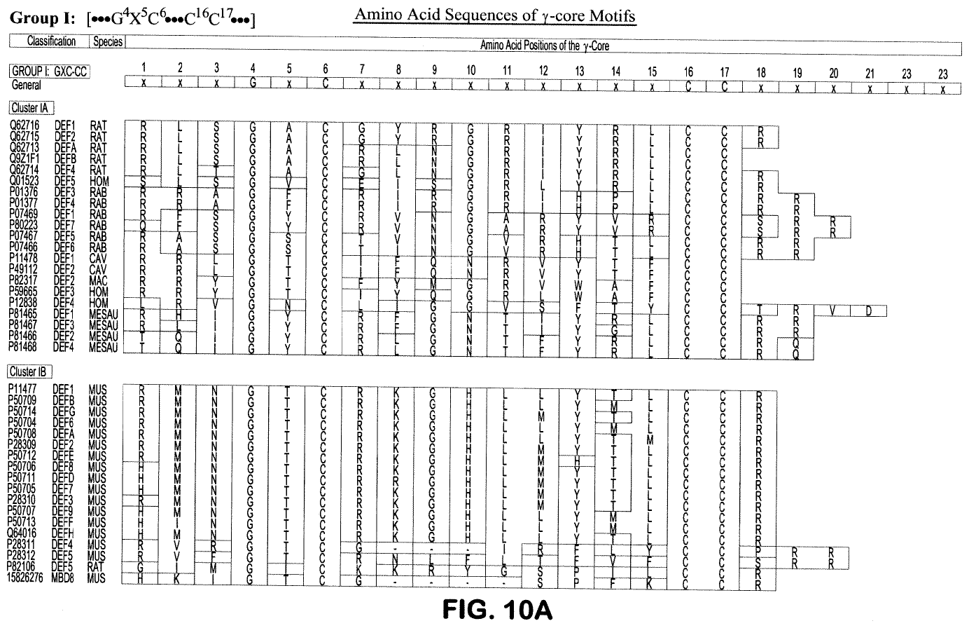

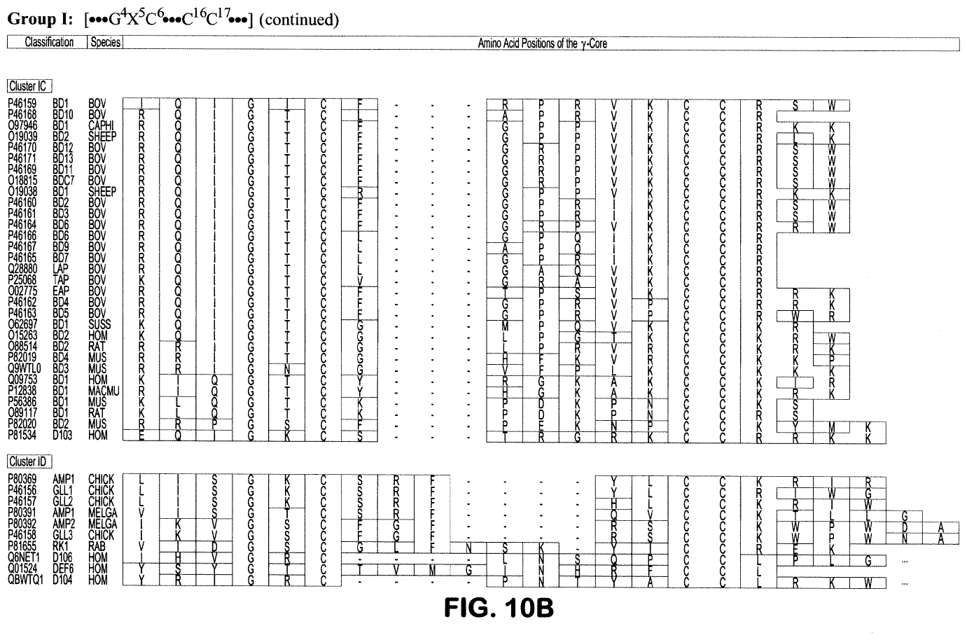

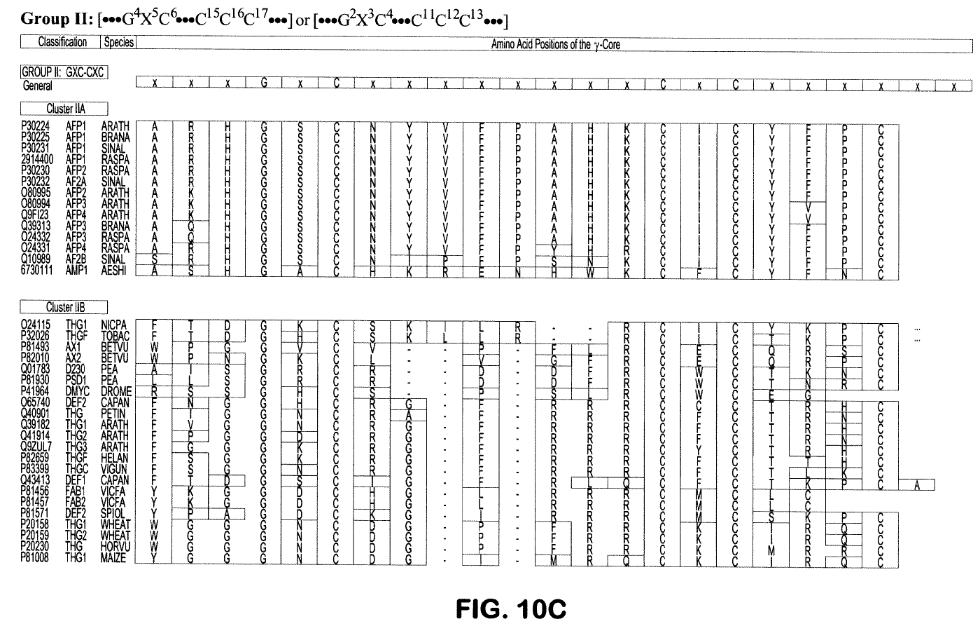

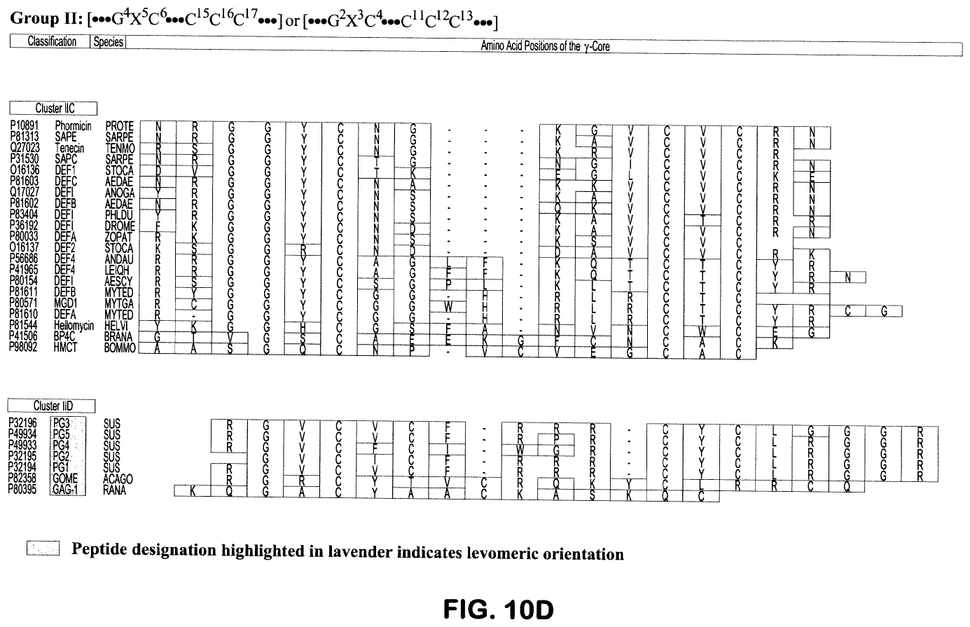

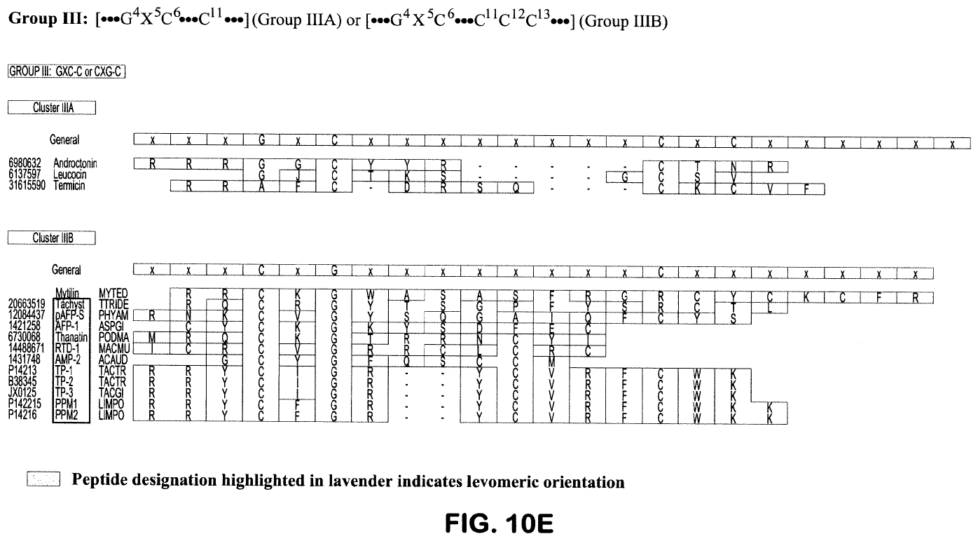

Conservation of the γ-core motif across the phylogenetic spectrum demonstrates it is an archetype of the antimicrobial peptide signature (FIG. 8A). Yet, the γ-core is not necessarily an exclusive structural determinant of antimicrobial activity. In some cases, the γ-core alone is sufficient for antimicrobial activity (eg., protegrins, tachyplesins, RTD-1). However, the motif also can serve as a scaffold, to which complementary antimicrobial determinants (eg., α-helices or β-sheets) are added as adjacent modules.

Thus, disulfide-stabilized antimicrobial peptides represent structural modules coordinated in varying configurations relative to the γ-core (FIG. 8B). Examples of the invention discovery are abundant in nature: Protegrin-1 illustrates the simplest configuration, consisting solely of the γ-core, represented by the modular formula [γ]; MGD-1 contains an α-helical module linked to a γ-core, collectively represented as [γ-α]; alternatively, HNP-3 exemplifies the addition of a β-sheet module to the γ-core, represented as [β-γ]; Ah-AMP-1 illustrates a more complex configuration in which β-sheet and α-helical modules are linked to the γ-core, represented by the formula [β-γ-α]. Permutations of these modular formulae are readily observed in naturally-occurring antimicrobial peptides, encompassing diverse antimicrobial peptide families, including α-defensins, β-defensins, θ-defensins, cathelicidins, protegrins, and CS-αβ peptides found in plants, invertebrates, insects, and arthropods. Based on this discovery the present invention provides methods of utilizing specific mosaic configurations of such structural modules to optimize the function of a given antimicrobial peptide against relevant pathogens in specific physiologic contexts.

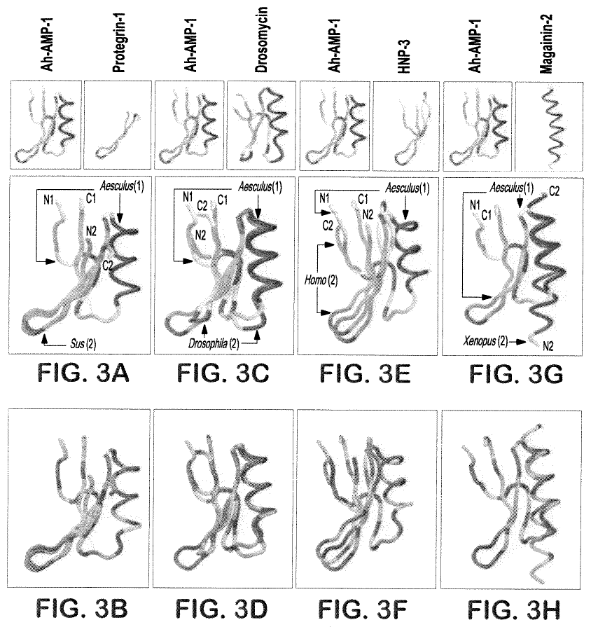

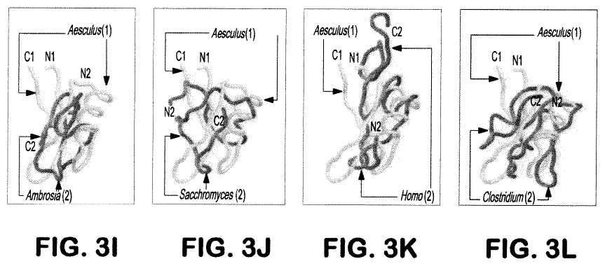

Thus, peptides with common evolutionary precursors may have conserved structural elements independent of functional divergence. As one verification of this discovery, AFP-1 and TGF-α were intentionally included in the exemplified phylogenetic and structural analyses as relative outliers in the comparative antimicrobial and non-antimicrobial peptide groups. This level of divergence is reflected in their significant phylogenetic distances from other peptides in their respective subsets. Yet, as described herein, despite equidistant divergence from Ah-AMP-1, AFP-1 exhibits the fundamental γ-core signature of antimicrobial peptides, while TGF-α does not (FIGS. 3 I-L and 8A). This result reinforces the importance of the γ-core motif as part of a multidimensional signature for antimicrobial activity. Moreover, structural divergence of AFP-1 from other antimicrobial peptides lies predominantly in modules beyond the γ-core. Thus, as exemplified for AFP-1, the invention provides new insights into eukaryotic evolution of the multidimensional signature of antimicrobial peptides that confer survival advantages in environments rich in microbial pathogens.

The discovery of a multidimensional signature as described herein can be applied to a method of identifying peptides that exert previously unrecognized antimicrobial activity. As described herein, for example, the sweetener protein, brazzein, and the scorpion neurotoxin, charybdotoxin, were found to have previously unrecognized antimicrobial activity against bacteria and fungi. The present model also accurately predicted that the prototype metallothionein II, which fulfilled the primary sequence pattern, but lacked the 3-dimensional criteria of the antimicrobial signature, was devoid of antimicrobial activity. As described herein, the multidimensional signature model was further substantiated by successful prediction of the γ-core motif in tachyplesins of unknown 3-dimensional structure, but which had known antimicrobial activity, and fulfilled the primary structure criteria of the model. Together, these findings validate the predictive accuracy, utility and applicability of the multidimensional antimicrobial peptide signature model to the methods provided by the present invention.

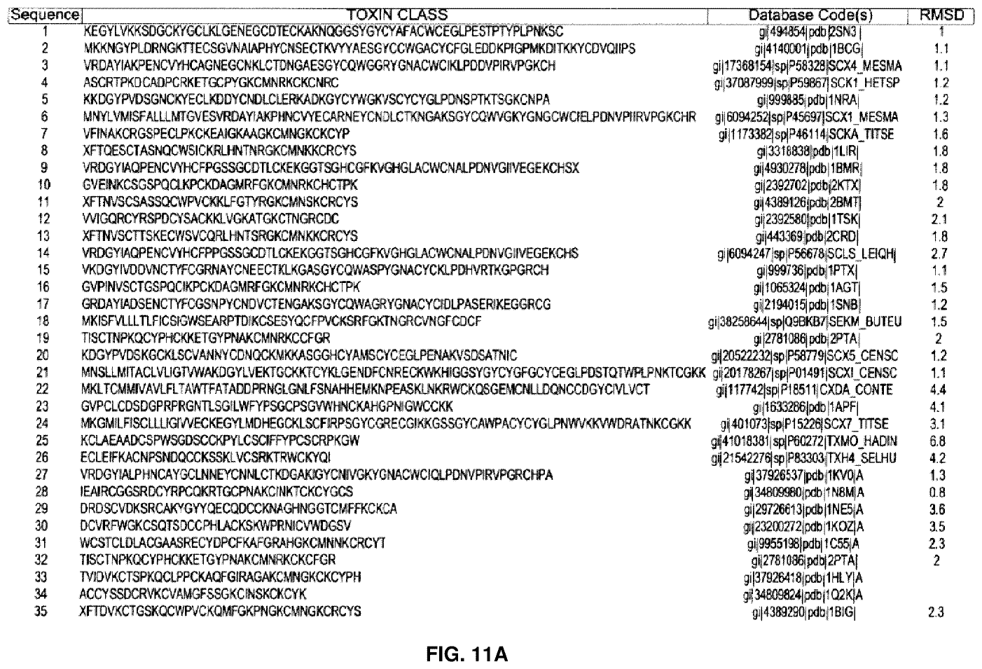

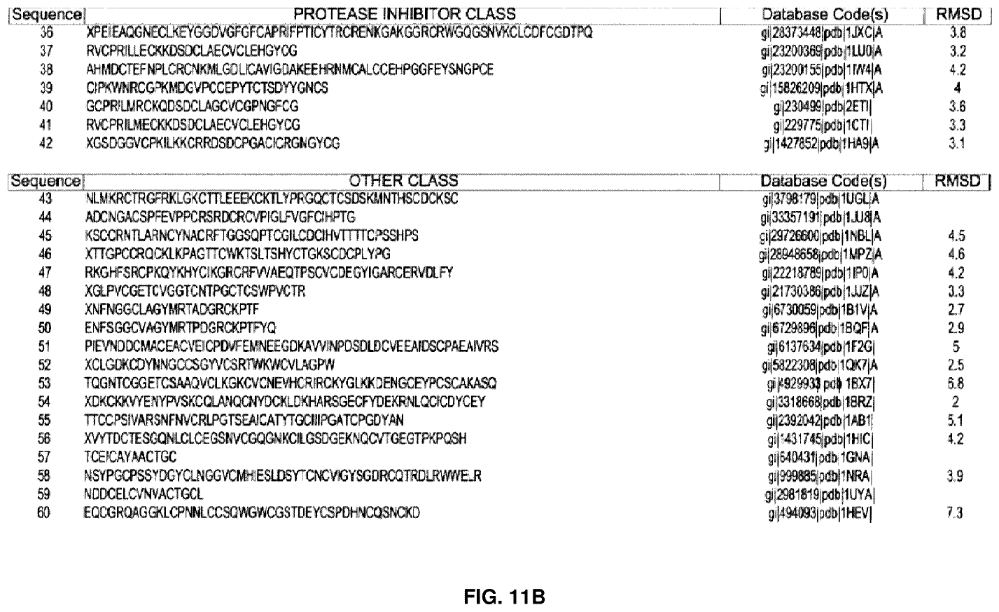

As disclosed herein, the multidimensional signature is a unifying structural code for broad classes of host defense peptides. This discovery is supported, for example, in the exemplification that a major class of peptides can be retrieved from the protein database searches using the stereospecific sequence formulae consisting of protease inhibitors and related proteins derived from plants (FIG. 11B). The botanical and related literature indicate that several such peptides have been shown to be plant defensins (Sallenave, Biochem. Soc. Trans. 30:111 (2002); Wijaya et al., Plant Sci 159:243 (2000)). Moreover, the plant proteinase inhibitor superfamily includes thionin peptides containing the antimicrobial γ-core motif as disclosed herein (Table I; Melo et al., Proteins 48:311 (2002)). In addition, peptides originally identified as having cytokine bioactivities are now known to have direct antimicrobial activity. Examples include γ-chemokines such as human platelet factor-4 and platelet basic peptide (PF-4 and PBP; Tang et al., Infect. Immun. 70:6524 (2002); Yeaman, Clin. Infect. Dis. 25:951 (1997)), monokine induced by interferon-γ (MIG/CXCL9; Cole et al., J. Immunol. 167:623 (2001)), interferon-γ inducible protein-10 kDa (IP-10/CXCL10; Cole et al., J. Immunol. 167:623 (2001)), interferon-inducible T cell α chemoattractant (ITAC/CXCL11; Cole et al., J. Immunol. 167:623 (2001)), and the β-chemokine, RANTES (releasable upon activation normal T cell expressed/secreted; Tang et al., Infect. Immun. 70:6524 (2002); Yeaman, Clin. Infect. Dis. 25:951 (1997)). Importantly, each of these proteins contains an iteration of the multidimensional antimicrobial signature as provided by the present invention. Collectively, these observations demonstrate the link between the multidimensional antimicrobial signature, and functional correlates in multifunctional host defense peptides (Yeaman, Clin. Infect. Dis. 25:951 (1997); Ganz, Science 298:977 (2002)). The skilled person will appreciate that the multidimensional antimicrobial signature can be found in additional peptides, and that the presence of this signature is associated with antimicrobial activity.

Multidimensional signatures of antimicrobial peptides exemplify how nature can diverge at the level of overall amino acid sequence, yet preserve essential primary sequence patterns and 3-dimensional determinants effective in host defense. Thus, critical structures of antimicrobial peptides from evolutionarily distant organisms such as microbes and plants are recapitulated in higher organisms, including humans. As disclosed herein, vertical and horizontal acquisition of genes, along with their recombination, yield mosaic iterations upon key structural determinants, such as the γ-core motif (Bevins et al., Genomics 31:95 (1996); Gudmundsson, et al., Proc. Natl. Acad. Sci. USA 92:7085 (1995)). Selective pressures favoring this remarkable degree of structural conservation can include genetic selection against structural variants, and convergent evolution of independent ancestral templates. It follows that the γ-core signature is incorporated into a variety of structural mosaics (eg., [γα₁], [γβ₁], or [γα₁β₁]) readily observed amongst disulfide-stabilized antimicrobial peptides along the phylogenetic spectrum. While future studies will resolve their precise phylogenetic lineage, the multidimensional signatures in antimicrobial peptides likely reflect fundamental host-pathogen interactions and their co-evolution.

The discovery and characterization of antimicrobial peptide signatures can also provide insights for development of new generation anti-infective agents. For example, most microbial pathogens are unable to acquire rapid or high-level resistance to antimicrobial peptides. Critical structure-activity relationships in these molecules can circumvent microbial resistance mechanisms, and interfere with essential microbial targets distinct from classical antibiotics (Yeaman and Yount, Pharmacol. Rev. 55:27 (2003)). Such modes of action exploit pathogen-specific structures intrinsically difficult to mutate, limiting the development of resistance through target or pathway modification. Thus, structural signatures in antimicrobial peptides can advance the discovery and development of improved anti-infective agents and strategies that are refractory to microbial resistance. Therefore, the invention provides a method of improving the antimicrobial activity of a protein by altering the multidimensional signature. Methods of protein design are well known in the art as described, for example, in Concepts in Protein Engineering and Design: An Introduction; Wrede and Schneider (Eds.), Walter de Gruyter, Inc. (pub.), 1994); Evolutionary Approaches to Protein Design, Vol. 55, Frances H. Arnold (Ed.), Edward M. Scolnick (Ed.), Elsevier Science & Technology Books, 2000; Molecular Design and Modeling: Concepts and Applications, Part A: Proteins, Peptides, and Enzymes: Volume 202: Molecular Design and Modelling Part A, John N. Abelson (Ed.), John J. Langone (Ed.), Melvin I. Simon (Ed.), Elsevier Science & Technology Books, 1991; and Protein Engineering and Design, Paul R. Carey (Ed.), Elsevier Science & Technology Books, 1996; all of which are incorporated herein by reference in their entirety.

While chemokines have not traditionally been ascribed with direct antimicrobial activities, evidence for such functions is mounting. As described above, peptides originally identified as having cytokine bioactivities, including chemokines platelet factor-4 (PF-4) platelet basic peptide (PBP) and its derivative CTAP-3 (Tang et al. (2002) Infect Immun 70, 6524-33; Yeaman et al. (1997) Infect Immun 65, 1023-31; Yount et al. (2004) Antimicrob Agents Chemother 48, 4395-404), as well as truncations thereof (Krijgsveld et al. (2000) J Biol Chem 275, 20374-81.), are now known to have direct antimicrobial activity. Direct antimicrobial activity was subsequently reported for other chemokines (Cole et al. (2001) J Immunol 167, 623-7; Yang et al. (2003) J Leukoc Biol 74, 448-55.). Hence, the term kinocidin (kino—action; cidin—microbicidal) has been applied to chemokines that also exert direct microbicidal activity (Yount & Yeaman (2004) Proc Natl Acad Sci U S A 101, 7363-8; Yount et al. (2004) Antimicrob Agents Chemother 48, 4395-404, Yeaman, M. R. & Yount, N. (2005) ASM News 71, 21-27.).

Despite immunological likeness with other kinocidins, prior investigations have not detected direct antimicrobial activity of IL-8. As described herein, primary sequence and conformation analyses specified IL-8 and kinocidin iterations of the γ-core signature present in broad classes of antimicrobial peptides (Yount & Yeaman (2004) Proc Natl Acad Sci U S A 101). Based on these structural parallels, IL-8 was discovered to have direct antimicrobial activity. Multiple lines of investigation described herein confirmed that IL-8 exerts significant, context-specific antimicrobial activity, with potent efficacy against Candida albicans. Moreover, the invention provides a synthetic congener corresponding to the α-helical domain of IL-8 that exerts antimicrobial activity equivalent to or exceeding that of native IL-8.

As disclosed herein, IL-8 and other kinocidins share key properties with classical antimicrobial peptides. For example, kinocidins exhibit global as well as local amphipathic domains, and have pI values of 8.5 or greater, indicating net positive charge at neutral pH (Table III). However, distribution of charge within kinocidins is not uniform; typically, cationic charge is associated with the C-terminal α-helix, and termini of the IL-8γ core. Molecular modeling has suggested these regions form electropositive facets of varying size in distinct kinocidins (Yang et al. (2003) J Leukoc Biol 74, 448-55). It is notable that, while study kinocidins share such cationic properties, neither net charge pI directly correlated with antimicrobial activity. For example, although IL-8 was one of the least cationic kinocidins studied (pI=9.0), it had greater efficacy than many of its more positively charged counterparts.

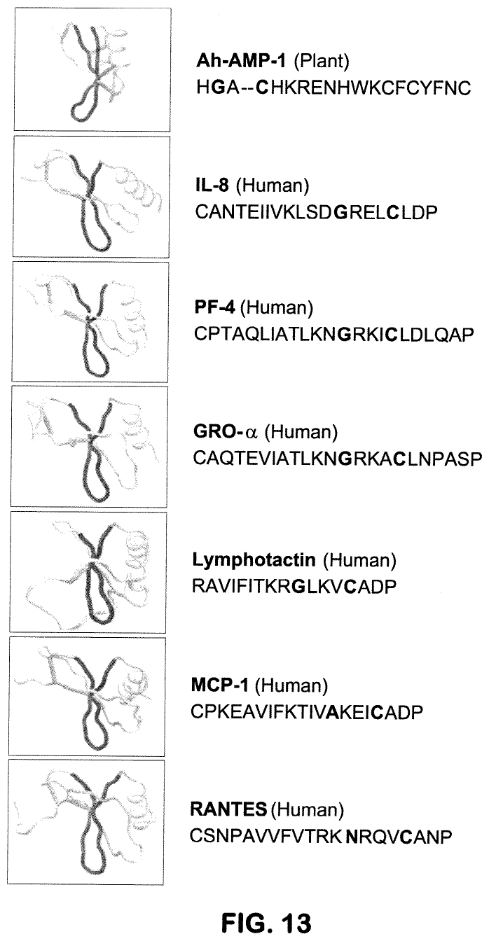

Recognition that charge alone does not account for antimicrobial activity emphasizes the importance of 3-D structure for kinocidin function. Notably, the strongly anti-fungal kinocidin IL-8 bears a striking structural resemblance to classical antifungal CS-αβ antimicrobial peptides of plants and insects (FIG. 13; Yount & Yeaman (2004) Proc Natl Acad Sci U S A 101). Kinocidins including IL-8 share a common topology comprised of a γ_{KC} core and α-helix. Likewise, CS-αβ family antimicrobial peptides also contain a γ_{AP} core and α-helix (Yang et al. (2003) J Leukoc Biol 74, 448-55). However, as kinocidins are larger (8-14 kDa) than many classical antimicrobial peptides (<5 kDa), it follows that global physicochemical properties do not strictly correlate with antimicrobial efficacy. Rather, it is more likely that discrete domains likely confer microbicidal versus chemotactic activities for kinocidins or chemotactic antimicrobial peptides (Yeaman & Yount (2005) ASM News 71, 21-27).

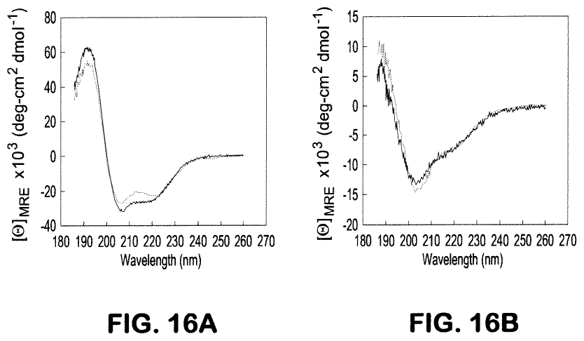

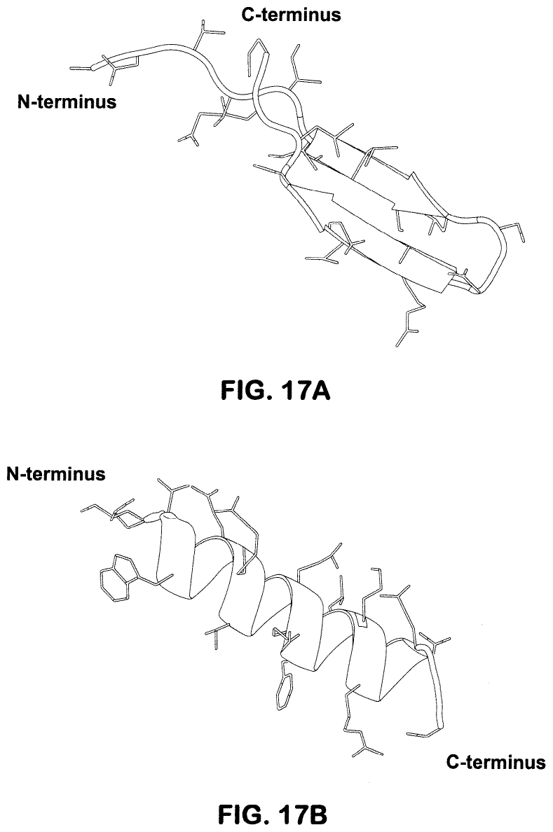

The fact that the C-terminal IL-8α peptide recapitulated the microbicidal efficacy and spectrum of native IL-8 substantiates the hypothesis that kinocidin antimicrobial effects can be mediated by specific or even autonomous structural domains. Inspection of the IL-8α domain revealed a highly structured helix, with physiochemical features likely attributable to its direct antimicrobial efficacy. Moreover, the current structure analyses concur with independent NMR studies (Clore et al. (1990) Biochemistry 29, 1689-96.) of the IL-8 α-helix domain. In addition, as its helical conformation is stable at pH 5.5 and 7.5, pH-specific antimicrobial efficacy of IL-8α relies on parameters other than conformation (FIG. 16). Superimposed upon the 19-residue IL-8α domain are cationic charge and amphipathicity (FIG. 17). Its estimated pI of 10 and net charge of +2 (at pH 7) indicate a degree of cationic potential greater than that of corresponding domains in other study kinocidins except lymphotactin. Furthermore, its amphipathicity (6.70; Table 3) is also relatively high, as hydrophobic moments >3.0 are considered significant in terms of hydrophobic versus hydrophilic amino acid segregation. Such characteristics parallel those in well-characterized helical antimicrobial peptides (Wieprecht et al. (1997) Biochemistry 36, 6124-32; Uematsu & Matsuzaki. (2000) Biophys J 79, 2075-83.).

Analyses of the physicochemical properties reveal that kinocidin molecules share key properties with classical antimicrobial peptides. For example, kinocidins exhibit global as well as local amphipathic domains, and have pI values of 8.5 or greater, indicating net positive charge at neutral pH (Table III). However, distribution of charge within kinocidins is not uniform; typically, cationic charge is associated with the C-terminal α-helix, and termini of the IL-8γ core. Molecular modeling has suggested these regions form electropositive facets of varying size in distinct kinocidins (Yang et al. (2003) J Leukoc Biol 74, 448-55).

The current results demonstrate direct antimicrobial activities of these and other kinocidins against bacteria in solid-phase and solution-phase assays, including striking fungicidal activity versus C. albicans. The surprising discovery of IL-8 antimicrobial efficacy may be due to a number of factors, including organism and/or strain differences, as well as buffer composition and pH. Prior studies using a radial diffusion assay measured activity against different organisms (Escherichia coli, Listeria monocytogenes), and did not assess activity at pH 5.5 (11). In the one prior study using a solution-phase assay, activity of IL-8 was evaluated against E. coli and S. aureus using a phosphate-based buffer system at pH 7.4 (Yang et al. (2003) J Leukoc Biol 74, 448-55). Results from these latter experiments corroborate the present finding that native IL-8 lacks activity against S. typhimurium or S. aureus in solution phase at pH 7.5.

The current investigations demonstrated that IL-8 exerted significant microbicidal efficacy at concentrations descending to the high nM range. While such concentrations reflect relatively strong microbicidal efficacy, it could be argued that even μg/ml levels of activity have limited physiologic relevance. However, several considerations support the concept that the antimicrobial effects of kinocidins including IL-8 observed in vitro are relevant to host defense in vivo. In normal human plasma, IL-8 is present at a very low baseline level in the range of picograms/ml (30). However, in contexts of infection, circulating IL-8 levels rise rapidly and dramatically as much as 1000-fold, yielding concentrations of 30-50 ng/ml (Moller et al. (2005) J Infect Dis 191, 768-75.). In the current report, IL-8 was active in the 5000-1000 ng/ml range, 100-fold greater than the highest measured concentrations in plasma. Yet, the potential for IL-8 and other kinocidins to reach efficacious concentrations in local contexts of infection is supported by considerable evidence. For example, recent studies by Qiu et al. show that the chemokine CCL22/MDC reaches μg/g levels in lung granulomae (Qiu et al. (2001) Am J Pathol 158, 1503-15). Additionally, as kinocidins adhere readily to pathogens, measurements of their free concentration diluted in media or sera almost certainly underestimate their local intensification (Mezzano et al. (1992) Nephron 61, 58-63.). Also, the systemic administration of α-helical antimicrobial peptides do not preclude their concentration specifically at sites of infection (Nibbering et al. (2004) J Nucl Med 45, 321-6.), perhaps by affinity of the cationic peptide for electronegative bacterial cell membranes. Such events likely achieve local concentrations of IL-8 and other kinocidins sufficient for microbicidal potency and chemotactic navigation.

In many contexts of infection or inflammation, pH of interstitial fluids, abscess exudates, and serum is significantly lower than that of plasma. Furthermore, recurring host-defense strategies include mild acidification of mucosal epithelia and the neutrophil phagolysosome. Thus, assessment of IL-8 and subdomain antimicrobial efficacy at pH 7.5 versus 5.5 was designed to reflect such microenvironments. The fact that kinocidins, including IL-8 and the IL-8α antimicrobial domain, exert enhanced antimicrobial efficacy at pH 5.5 is consistent with these concepts. Thus, beyond providing a chemical barrier, such pH modulation may contribute to mucosal surfaces that are inhospitable to microbial colonization. A parallel line of reasoning also supports the concept that kinocidins mutually potentiate the antimicrobial mechanisms of leukocytes. Kinocidins are known to interact with leukocytes via chemokine motifs, and with microorganisms via charge-mediated properties (Yang et al. (2003) J Leukoc Biol 74, 448-55). Thus, pathogens pre-decorated with kinocidins or antimicrobial domains thereof are believed to be more efficiently killed when internalized into the acidic phagolysosome of professional phagocytes (5). Additional support for this concept is exemplified by studies demonstrating significant quantities of the kinocidin PBP in the phagolysosomes of activated macrophages (34). In these ways, kinocidins are likely evolved to function in specific contexts to optimize antimicrobial defenses without concomitant host toxicity.

Peptides useful as antifungal or antibacterial agents are those which are at least 5 or more, 6 or more, 7 or more, 8 or more, 9 or more, 10 or more, 11 or more, 12 or more, 13 or more, 14 or more, 15 or more amino acids in length and which comprise at least a portion of the alpha-helical structure sufficient for antimicrobial activity, and substitutions thereof. An example of a permissible substitutions is alanine for cysteine.

The peptides of the present invention can be chemically synthesized. Thus polypeptides can be prepared by solid phase peptide synthesis, for example as described by Merrifield. The synthesis is typically carried out with amino acids that are protected at the alpha-amino terminus. Trifunctional amino acids with labile side-chains are also protected with suitable groups to prevent undesired chemical reactions from occurring during the assembly of the polypeptides. The alpha-amino protecting group is selectively removed to allow subsequent reaction to take place at the amino-terminus. The conditions for the removal of the alpha-amino protecting group do not remove the side-chain protecting groups.

The alpha-amino protecting groups are well known to those skilled in the art and include acyl type protecting groups (e.g., formyl, trifluoroacetyl, acetyl), aryl type protecting groups (e.g., biotinyl), aromatic urethane type protecting groups [e.g., benzyloxycarbonyl (Cbz), substituted benzyloxycarbonyl and 9-fluorenylmethyloxy-carbonyl (Fmoc)], aliphatic urethane protecting groups [e.g., t-butyloxycarbonyl (tBoc), isopropyloxycarbonyl, cyclohexloxycarbonyl] and alkyl type protecting groups (e.g., benzyl, triphenylmethyl). The preferred protecting groups are tBoc and Fmoc.

The side-chain protecting groups selected must remain intact during coupling and not be removed during the deprotection of the amino-terminus protecting group or during coupling conditions. The side-chain protecting groups are also removable upon the completion of synthesis using reaction conditions that will not alter the finished polypeptide. In tBoc chemistry, the side-chain protecting groups for trifunctional amino acids are mostly benzyl based. In Fmoc chemistry, they are mostly tert-butyl or trityl based.

In addition, the peptides can also be prepared by recombinant DNA technologies wherein host cells are transformed with proper recombinant plasmids containing the nucleotide sequence encoding the particular peptide. The peptides of the present invention can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are well known in the art and can be found in standard references as Sambrook et al., Molecular Cloning: A Laboratory Manual, Third Ed., Cold Spring Harbor Laboratory, New York (2001) and Ansubel et al., Current Protocols in Molecular Biology, John Wiley and Sons, Baltimore, MD (1999), both of which are incorporated herein by reference.

In general, a DNA sequence encoding a peptide of the present invention is operably linked to other genetic elements required for its expression, generally including a transcription promoter and terminator within an expression vector. The vector typically contains one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are available through commercial suppliers.

The peptides of the present invention can be formulated into compositions in pharmaceutically acceptable carriers for administration to individuals. For oral administration, the peptides can be formulated into a solid preparation such as tablets, pills, granules, powder, capsules and the like, or a liquid preparation such as solutions, suspensions, emulsions and the like. The pharmaceutical preparations for oral administration comprising one or more peptides of the present invention may also contain one or more of the following customary excipients: fillers and extenders including starches, lactose, sucrose, glucose, mannitol and silica; binders including carboxymethylcellulose, alginates, gelatine and polyvinylpyrrolidone; humectants including glycerine; disintegrating agents, including agar-agar, calcium carbonate and sodium carbonate; solution retarders, including paraffin; absorption accelerators including quaternary ammonium compound; wetting agents including cetyl alcohol or glycerine monostearate; adsorbents including kaolin and bentonite; lubricants including talc, calcium stearate and magnesium stearate and solid polyethylene glycols; colorants; flavorings; and sweeteners.

When the preparation is used for parental administration, the preparation is made in an injection formula. For the preparation of an injection formula, the solutions and emulsions can be in a sterile form which is isotonic with blood. The suspensions can contain in addition to the active peptide or peptides, preservatives, stabilizers, solubilizers, wetting agents, salts for changing the osmotic pressure or buffers.

The peptides of the present invention are useful as antifungal or antibacterial agents.

The invention provides methods of using kinocidin peptide constructs such as IL-8α for treating a subject suffering from infection (including fungal, bacterial, or other microbial infection), especially mammalian subjects such as humans, but also including farm animals such as cows, sheep, pigs, horses, goats and/or poultry (e.g., chickens, turkeys, ducks and/or geese), companion animals such as dogs and/or cats, exotic and/or zoo animals, and/or laboratory animals including mice, rats, rabbits, guinea pigs, and/or hamsters. Immunocompromised or immunosuppressed subjects, e.g., subjects suffering from cancer, subjects undergoing radiation therapy and/or cytotoxic chemotherapy, subjects being treated with immunosuppressive drugs, and/or subjects suffering from natural or acquired immune deficiencies such as AIDS, may be treated according to this aspect of the invention. Treatment of infection of plants is also contemplated.

“Treatment” as used herein encompasses both prophylactic and/or therapeutic treatment, and may be accompanied by concurrent administration of other antimicrobial agents, including any of the agents discussed herein.

Fungal infection that may be treated according to the invention may be caused by a variety of fungal species including Candida (including C. albicans, C. tropicalis, C. parapsilosis, C. stellatoidea, C. krusei, C. parakrusei, C. lusitanae, C. pseudotropicalis, C. guilliermondi, C. dubliniensis, C. famata or C. glabrata), Aspergillus (including A. fumigatus, A. flavus, A. niger, A. nidulans, A. terreus, A. sydowi, A. flavatus, or A. glaucus), Cryptococcus, Histoplasma, Coccidioides, Paracoccidioides, Blastomyces, Basidiobolus, Conidiobolus, Rhizopus, Rhizomucor, Mucor, Absidia, Mortierella, Cunninghamella, Saksenaea, Pseudallescheria, Paecilomyces, Fusarium, Trichophyton, Trichosporon, Microsporum, Epidermophyton, Scytalidium, Malassezia, Actinomycetes, Sporothrix, Penicillium, Saccharomyces or Pneumocystis.

Other infections that may be treated using a peptide construct according to the invention may be caused by gram-negative bacterial species that include Acidaminococcus, Acinetobacter, Aeromonas, Alcaligenes, Bacteroides, Bordetella, Branhamella, Brucella, Burkholderia, Calymmatobacterium, Campylobacter, Cardiobacterium, Chromobacterium, Citrobacter, Edwardsiella, Enterobacter, Escherichia, Flavobacterium, Francisella, Fusobacterium, Haemophilus, Klebsiella, Legionella, Moraxella, Morganella, Neisseria, Pasturella, Plesiomonas, Porphyromonas, Prevotella, Proteus, Providencia, Pseudomonas, Salmonella, Serratia, Shigella, Stentrophomonas, Streptobacillus, Treponema, Veillonella, Vibrio, or Yersinia species; Chlamydia; or gram-positive bacterial species that include Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Nocardia, Actinomyces, or Corynebacterium species as well as Mycoplasma, Ureaplasma, or Mycobacteria.

Other infections include infections by protozoa including Plasmodia, Toxoplasma, Leishmania, Trypanosoma, Giardia, Entamoeba, Acanthamoeba, Nagleria, Hartmanella, Balantidium, Babesia, Cryptosporidium, Isospora, Microsporidium, Trichomonas or Pneumocystis species; or infections by other parasites include helminths.

Other therapeutic uses of kinocidin peptide constructs such as IL-8α according to the invention include methods of treating conditions associated with endotoxin, such as exposure to gram-negative bacterial endotoxin in circulation, endotoxemia, bacterial and/or endotoxin-related shock and one or more conditions associated therewith, including a systemic inflammatory response, cytokine overstimulation, complement activation, disseminated intravascular coagulation, increased vascular permeability, anemia, thrombocytopenia, leukopenia, pulmonary edema, adult respiratory distress syndrome, renal insufficiency and failure, hypotension, fever, tachycardia, tachypnea, and metabolic acidosis. Thus, not only gram-negative bacterial infection but also conditions which are associated with exposure to gram-negative bacterial endotoxin (infection-related conditions) may be ameliorated through endotoxin-binding or endotoxin-neutralizing activities of kinocidin peptide constructs such as IL-8α.

Therapeutic compositions of the peptide construct may include a pharmaceutically acceptable diluent, adjuvant, or carrier. The peptide construct may be administered without or in conjunction with known surfactants, other chemotherapeutic agents or additional known antimicrobial agents.

Compositions, including therapeutic compositions, of the peptide construct of the invention may be administered systemically or topically. Systemic routes of administration include oral, intravenous, intramuscular or subcutaneous injection (including into depots for long-term release), intraocular or retrobulbar, intrathecal, intraperitoneal (e.g. by intraperitoneal lavage), intrapulmonary (using powdered drug, or an aerosolized or nebulized drug solution), or transdermal. Topical routes include administration in the form of rinses, washes, salves, creams, jellies, drops or ointments (including opthalmic and otic preparations), suppositories, such as vaginal suppositories, or irrigation fluids (for, e.g., irrigation of wounds).

Suitable dosages include doses ranging from 1 mg/kg to 100 mg/kg per day and doses ranging from 0.1 mg/kg to 20 mg/kg per day. When given parenterally, compositions are generally injected in one or more doses ranging from 1 mg/kg to 100 mg/kg per day, preferably at doses ranging from 0.1 mg/kg to 20 mg/kg per day, and more preferably at doses ranging from 1 to 20 mg/kg/day. As described herein for compositions with IL-8α, parenteral doses of 0.5 to 5 mg/kg/day are preferred according to the present invention. The treatment may continue by continuous infusion or intermittent injection or infusion, or a combination thereof, at the same, reduced or increased dose per day for as long as determined by the treating physician. An antimicrobial composition can be effective at blood serum concentrations as low as 1 μg/ml. When given topically, compositions are generally applied in unit doses ranging from 1 mg/mL to 1 gm/mL, and preferably in doses ranging from 1 mg/mL to 100 mg/mL. Decontaminating doses are applied including, for example, for fluids or surfaces or to decontaminate or sterilize surgical or other medical equipment or implantable devices, including, for example prosthetic joints or in indwelling invasive devices. Those skilled in the art can readily optimize effective dosages and administration regimens for therapeutic, including decontaminating, compositions as determined by good medical practice and the clinical condition of the individual subject.

“Concurrent administration,” or “co-administration,” as used herein includes administration of one or more agents, in conjunction or combination, together, or before or after each other. The agents maybe administered by the same or by different routes. If administered via the same route, the agents may be given simultaneously or sequentially, as long as they are given in a manner sufficient to allow all agents to achieve effective concentrations at the site of action. For example, a peptide construct may be administered intravenously while the second agent(s) is(are) administered intravenously, intramuscularly, subcutaneously, orally or intraperitoneally. A peptide construct and a second agent(s) may be given sequentially in the same intravenous line or may be given in different intravenous lines. Alternatively, a peptide construct may be administered in a special form for gastric or aerosol delivery, while the second agent(s) is(are) administered, e.g., orally.

Concurrent administration of the peptide construct of the invention, such as IL-8α, for adjunctive therapy with one or more other antimicrobial agents (particularly antifungal agents) is expected to improve the therapeutic effectiveness of the antimicrobial agents. This may occur through reducing the concentration of antimicrobial agent required to eradicate or inhibit target cell growth, e.g., replication. Because the use of some antimicrobial agents is limited by their systemic toxicity, lowering the concentration of antimicrobial agent required for therapeutic effectiveness reduces toxicity and allows wider use of the agent. For example, concurrent administration of the peptide construct, such as IL-8α, and another antifungal agent may produce a more rapid or complete fungicidal or fungistatic effect than could be achieved with either agent alone. Administration of the peptide construct, such as IL-8α, may reverse the resistance of fungi to antifungal agents or may convert a fungistatic agent into a fungicidal agent. Similar results may be observed upon concurrent administration of the peptide construct, such as IL-8α, with other antimicrobial agents, including antibacterial and/or anti-endotoxin agents.

Therapeutic effectiveness in vivo is based on a successful clinical outcome, and does not require that the antimicrobial agent or agents kill 100% of the organisms involved in the infection. Success depends on achieving a level of antimicrobial activity at the site of infection that is sufficient to inhibit growth or replication of the pathogenic organism in a manner that tips the balance in favor of the host. When host defenses are maximally effective, the antimicrobial effect required may be minimal. Reducing organism load by even one log (a factor of 10) may permit the host's own defenses to control the infection. In addition, augmenting an early microbicidal/microbistatic effect can be more important than a long-term effect. These early events are a significant and critical part of therapeutic success, because they allow time for host defense mechanisms to activate.

In addition, the invention provides a method of killing or inhibiting growth of pathogenic organisms (particularly fungi) comprising contacting the organism with the peptide construct, such as IL-8α, optionally in conjunction with other antimicrobial agents. This method can be practiced in vivo, ex vivo, or in a variety of in vitro uses such as to decontaminate fluids or surfaces or to sterilize surgical or other medical equipment or implantable devices, including prostheses orintrauterine devices. These methods can also be used for in situ decontamination and/or sterilization of indwelling invasive devices such as intravenous lines and catheters, which are often foci of infection.

A further aspect of the invention involves use of the peptide construct, such as IL-8α, for the manufacture of a medicament for treatment of microbial infection (e.g., fungal or bacterial infection) or a medicament for concurrent administration with another agent for treatment of microbial infection. The medicament may optionally comprise a pharmaceutically acceptable diluent, adjuvant or carrier and also may include, in addition to the kinocidin peptide construct, other chemotherapeutic agents.

Known antifungal agents which can be co-administered or combined with the kinocidin peptide construct according to the invention include polyene derivatives, such as amphotericin B (including lipid or liposomal formulations thereof) or the structurally related compounds nystatin or pimaricin; flucytosine (5-fluorocytosine); azole derivatives (including ketoconazole, clotrimazole, miconazole, econazole, butoconazole, oxiconazole, sulconazole, tioconazole, terconazole, fluconazole, itraconazole, voriconazole [Pfizer], posaconazole [SCH56592, Schering-Plough]) or ravuconazole [Bristol-Myers Squibb]; allylamines-thiocarbamates (including tolnaftate, naftifine or terbinafine); griseofulvin; ciclopirox; haloprogin; echinocandins (including caspofungin [MK-0991, Merck], FK463 [Fujisawa], cilofungin [Eli Lilly] or VER-002 [Versicor]); nikkomycins; or sordarins.

The polyene derivatives, which include amphotericin B or the structurally related compounds nystatin or pimaricin, are broad-spectrum antifungals that bind to ergosterol, a component of fungal cell membranes, and thereby disrupt the membranes. Amphotericin B is usually effective for systemic mycoses, but its administration is limited by toxic effects that include fever, kidney damage, or other accompanying side effects such as anemia, low blood pressure, headache, nausea, vomiting or phlebitis. The unrelated antifungal agent flucytosine (5-fluorocytosine), an orally absorbed drug, is frequently used as an adjunct to amphotericin B treatment for some forms of candidiasis or cryptococcal meningitis. Its adverse effects include bone marrow depression, including with leukopenia or thrombocytopenia.

Known antibacterial agents which can be co-administered or combined with the peptide construct according to the invention include antibiotics, which are natural chemical substances of relatively low molecular weight produced by various species of microorganisms, such as bacteria (including Bacillus species), actinomycetes (including Streptomyces) or fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics include (1) the β-lactams, including the penicillins, cephalosporins or monobactams, including those with β-lactamase inhibitors; (2) the aminoglycosides, e.g., gentamicin, tobramycin, netilmycin, or amikacin; (3) the tetracyclines; (4) the sulfonamides and/or trimethoprim; (5) the quinolones or fluoroquinolones, e.g., ciprofloxacin, norfloxacin, ofloxacin, moxifloxacin, trovafloxacin, grepafloxacin, levofloxacin or gatifloxacin (6) vancomycin; (7) the macrolides, which include for example, erythromycin, azithromycin, or clarithromycin; or (8) other antibiotics, e.g., the polymyxins, chloramphenicol, rifampin, the lincosamides, or the oxazolidinones.

Some drugs, for example, aminoglycosides, have a small therapeutic window. For example, 2 to 4 μg/ml of gentamicin or tobramycin may be required for inhibition of bacterial growth, but peak concentrations in plasma above 6 to 10 μg/ml may result in ototoxicity or nephrotoxicity. These agents are more difficult to administer because the ratio of toxic to therapeutic concentrations is very low. Antimicrobial agents that have toxic effects on the kidneys and that are also eliminated primarily by the kidneys, such as the aminoglycosides or vancomycin, require particular caution because reduced elimination can lead to increased plasma concentrations, which in turn may cause increased toxicity. Doses of antimicrobial agents that are eliminated by the kidneys must be reduced in patients with impaired renal function. Similarly, dosages of drugs that are metabolized or excreted by the liver, such as erythromycin, chloramphenicol, or clindamycin, must be reduced in patients with decreased hepatic function. In situations where an antimicrobial agent causes toxic effects, the kinocidin peptide construct, such as IL-8α, can act to reduce the amount of this antimicrobial agent needed to provide the desired clinical effect.

The susceptibility of a bacterial species to an antibiotic is generally determined by any art recognized microbiological method. A rapid but crude procedure uses commercially available filter paper disks that have been impregnated with a specific quantity of the antibiotic drug. These disks are placed on the surface of agar plates that have been streaked with a culture of the organism being tested, and the plates are observed for zones of growth inhibition. A more accurate technique, the broth dilution susceptibility test, involves preparing test tubes containing serial dilutions of the drug in liquid culture media, then inoculating the organism being tested into the tubes. The lowest concentration of drug that inhibits growth of the bacteria after a suitable period of incubation is reported as the minimum inhibitory concentration.

The resistance or susceptibility of an organism to an antibiotic is determined on the basis of clinical outcome, i.e., whether administration of that antibiotic to a subject infected by that organism will successfully cure the subject. While an organism may literally be susceptible to a high concentration of an antibiotic in vitro, the organism may in fact be resistant to that antibiotic at physiologically realistic concentrations. If the concentration of drug required to inhibit growth of or kill the organism is greater than the concentration that can safely be achieved without toxicity to the subject, the microorganism is considered to be resistant to the antibiotic. To facilitate the identification of antibiotic resistance or susceptibility using in vitro test results, the National Committee for Clinical Laboratory Standards (NCCLS) has formulated standards for antibiotic susceptibility that correlate clinical outcome to in vitro determinations of the minimum inhibitory concentration of antibiotic.

Other aspects and advantages of the present invention will be understood upon consideration of the following illustrative examples.

It is understood that modifications which do not substantially affect the activity of the various embodiments of this invention are also included within the definition of the invention provided herein. Accordingly, the following examples are intended to illustrate but not limit the present invention.

### Example I

### Identification of Multidimensional Signatures of Antimicrobial Peptides

This Example shows identification of a disulfide-stabilized core motif that is integral to the 3-dimensional signature of cysteine-containing antimicrobial peptides.

The relatedness amongst primary structures was examined in prototypic cysteine-containing antimicrobial peptide sequences representing taxa spanning an evolutionary distance of 2.6 billion years (BY; estimated date of phylogenetic divergence of fungi and plants from higher organisms; Nei et al., Proc. Natl. Acad. Sci. U S A. 98:2497 (2001)). A prototype from each class of non-cyclic, disulfide-containing antimicrobial peptides was represented in these analyses [Antimicrobial peptides were selected from the National Center for Biotechnology Information (NCBI) Entrez Protein (www.ncbi.nlm.nih.gov:80/entrez/) or Antimicrobial Sequences (www.bbcm.univ.trieste.it/˜tossi/) databases.]

The specific criteria for selection of peptides analyzed included: 1) eukaryotic origin; 2) published antimicrobial activity; 3) non-enzymatic mechanism(s) of action; 4) mature protein sequence; and 5) less than 75 amino acids in length. Peptides for which structures have been determined were used in structural analyses. [Peptides were selected from the National Center for Biotechnology Information (NCBI) structure (www.ncbi.nlm.nih.gov:80/entrez/) and Protein Data Bank (PDB) (www.rcsb.org/pdb/) resources.] The resulting study set included antimicrobial peptides encompassing a broad distribution in source (i.e., biological kingdoms ranging from microorganisms to man), amino acid sequence, and conformation class (FIG. 1). Amino acid sequence data were used for these analyses, as not all nucleotide sequences have been characterized, and saturation of nucleotide sequence data occurs within non-mitochondrial sequences over evolutionary timescales.

FIG. 1 shows conventional antimicrobial peptide structure classification and distribution. The relationship amongst structure and predominance is summarized for the commonly recognized antimicrobial peptide classes. Concatenation represents the proportionate distribution of peptides encompassing a given structural class, as calculated from the Antimicrobial Sequences Database. Antimicrobial peptides were selected from the National Center for Biotechnology Information (NCBI) Entrez Protein (www.ncbi.nlm.nih.gov:80/entrez/) or Antimicrobial Sequences (www.bbcm.univ.trieste.it/˜tossi/) databases. The numbers of peptides classified in each group are indicated in brackets for each class. Of the more than 750 peptides present in the database at the onset of the study, the balance of those not indicated are comprised of peptides representing unusual or other classifications, including macrocyclic, proline-rich, tryptophan-rich or indolicidin-like peptides, and large polypeptides greater than 75 amino acids in length.

Representatives included antimicrobial peptides from taxa encompassing broad biological diversity spanning an evolutionary distance of 2.6 billion years (estimated divergence of fungi and plants from higher organisms; [Nei et al, Proc. Natl. Acad. Sci. USA 98:2497 (2001).]). This dataset included prototypes of all major classes of disulfide-containing antimicrobial peptides, including distinct conformation groups such as defensin, cysteine-stabilized αβ, ranabox and β-hairpin.

Conventional MSA (N to C terminal; dextromeric) revealed no clear consensus patterns amongst primary sequences of the antimicrobial peptide study set. However, visual inspection revealed an absolutely conserved GXC motif, oriented in reverse in some peptides. We hypothesized that conventional MSA failed to recognize this inverted consensus pattern. Therefore, peptides containing inverted GXC motifs were aligned in their C to N terminal (levomeric) orientation. This stereospecific MSA revealed a novel and striking sequence pattern common to all disulfide-containing antimicrobial peptide classes (FIG. 2B). The consensus patterns, defined herein as the enantiomeric sequence signature, adhere to the formulae:

(dextromeric isoform)
(SEQ ID NO: 360)
NH₂ . . . [X₁₋₃]-[GXC]-[X₃₋₉]-[C] . . . COOH
(levomeric isoform 1)
(SEQ ID NO: 361)
NH₂ . . . [C]-[X₃₋₉]-[CXG]-[X₁₋₃] . . . COOH
(levomeric isoform 2)
(SEQ ID NO: 362)
NH₂ . . . [C]-[X₃₋₉]-[GXC]-[X₁₋₃] . . . COOH

These consensus patterns transcend defensin-specific motifs identified previously (White et al., Curr. Opin. Struct. Biol. 5:521 (1995); Yount et al., J. Biol. Chem. 274:26249 (1999). Specific characteristics of the enantiomeric sequence signatures include: i) a length of 8-16 amino acid residues; and ii) conserved GXC or CXG motifs within the sequence isoforms. Interestingly, levomeric isoform 2 peptides retain a dextromeric GXC motif within the levomeric sequence signature (FIG. 2B).

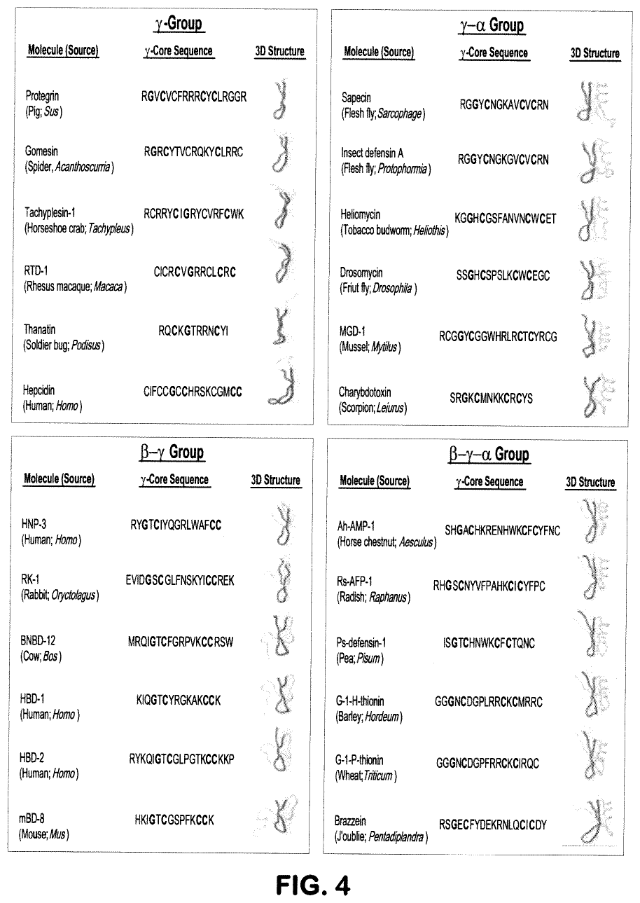

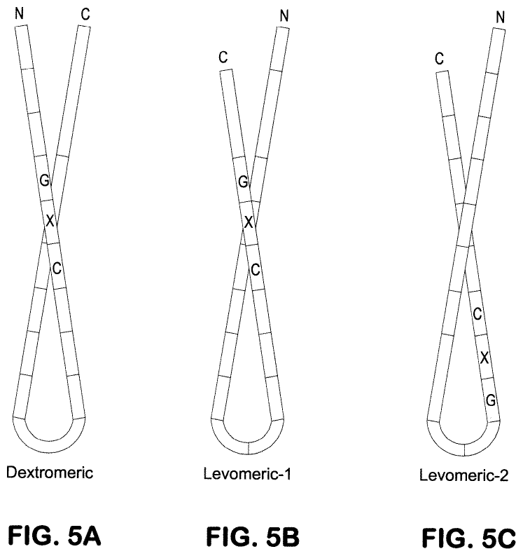

Identification of the conserved enantiomeric signature suggested that a corresponding motif would also be present in the 3-dimensional structures of disulfide-stabilized antimicrobial peptides. Conformation alignments revealed a core motif that was absolutely conserved across all classes of disulfide-stabilized antimicrobial peptides (FIG. 3A-H; Table 1). This 3-dimensional archetype, termed herein as the γ-core motif, is comprised of two anti-parallel β-sheets, interposed by a short turn region (FIGS. 4 and 5). All three isoforms of the enantiomeric sequence signature conform to the γ-core motif, reflecting their 3-dimensional convergence (FIG. 5). Additional features that characterize the γ-core include: 1) net cationic charge (+0.5 to +7) with basic residues typically polarized along its axis; 2) periodic charge and hydrophobicity yielding amphipathic stereogeometry; and 3) participation in 1-4 disulfide bonds. This motif may comprise the entire peptide, or link to adjacent structural domains.

Relative to the γ-core, disulfide-stabilized antimicrobial peptides of evolutionarily distant organisms exhibited a striking convergence in conformation, that was essentially isomeric, or at a minimum, highly homologous (FIG. 3). This 3-dimensional convergence encompassed overall conformations, or localized to specific domains in comparative peptides. For example, the structures of Ah-AMP-1 (horsechestnut tree, Aesculus) and drosomycin (fruit fly, Drosophila) are essentially superimposable over their entire backbone trajectories (FIG. 3C). Alternatively, protegrin-1 (domestic pig, Sus) and Ah-AMP-1 share conformational homology corresponding to their γ-core motifs (FIG. 3A). As anticipated, magainin aligned to the α-helical motif in Ah-AMP-1 (FIG. 3G), verifying the specificity of conformational alignments.

To confirm the significance of 3-dimensional convergence in the antimicrobial peptide signature, comparisons between representative cysteine-containing antimicrobial and non-antimicrobial peptides of equivalent molecular weight were performed and analyzed. Outcomes emphasize that non-antimicrobial peptides fail to achieve the multidimensional signature of antimicrobial peptides (FIG. 3I-L). Mean quantitative RMSD confirmed the statistical significance of the differences between antimicrobial and non-antimicrobial structures (Table II).

TABLE II
Quantitative analysis of 3-dimensional convergence amongst prototypic
antimicrobial peptide structures.

| | AAs | RMSD (Å) | Identity (%) | Align/Gap |
|---|---|---|---|---|
| Antimicrobial Peptides | | | | |
| Ah-AMP-1 (Aesculus; Tree; 1BK8; Wieprecht | 50 | 0.0 | 100 | 50/0 |
| et al. (1997) Biochemistry 36, 6124-32 ) | | | | |
| Sapecin (Sarcophaga; Fly; 1L4V; Uematsu, N. | 40 | 0.9 | 25.0 | 38/0 |
| & Matsuzaki, K. (2000) Biophys J 79, 2075-83 ) | | | | |
| Protegrin-1 (Sus; Pig; 1PG1; Fahrner et al., | 19 | 1.2 | 18.8 | 16/0 |
| Chem. Biol. 3: 543 (1996)) | | | | |
| Drosomycin (Drosophila; Fruit Fly; 1MYN; | 44 | 1.4 | 29.3 | 41/6 |
| Landon et al., Protein Sci. 6: 1878 (1997)) | | | | |
| Defensin (Raphanus; Radish; 1AYJ; Fant et al., | 51 | 1.3 | 47.6 | 49/0 |
| J. Mol. Biol. 279: 257 (1998)) | | | | |
| Thionin (Triticalis; Wheat; 1GPS; Bruix et al., | 47 | 1.8 | 26.1 | 46/3 |
| Biochemistry 32: 715 (1993)) | | | | |
| MGD-1 (Mytilus; Mussel; 1FJN; Yang et al., | 39 | 2.0 | 26.5 | 34/1 |
| Biochemistry 39: 14436 (2000)) | | | | |
| Thanatin (Podisus; Soldier Bug; 8TFV; | 21 | 2.2 | 12.5 | 16/0 |
| Mandard et al., Eur. J. Biochem. 256: 404 | | | | |
| (1998)) | | | | |
| HNP-3 (Homo; Human; 1DFN; Hill et al., | 34 | 3.2 | 8.3 | 24/17 |
| Science 251: 1481 (1991)) | | | | |
| MBD-8 (Mus; Mouse; 1E4R; Bauer et al., | 35 | 3.4 | 0.0 | 24/13 |
| Protein Sci. 10: 2470 (2001)) | | | | |
| AFP-1 (Aspergillus; Fungus; 1AFP; Campos- | 51 | 4.8 | 6.2 | 32/7 |
| Olivas et al., Biochemistry 34: 3009 (1995)) | | | | |
| Mean ± SD | | 2.2 ± 1.2* | | |
| Non-Antimicrobial Peptides | | | | |
| TGF-α (Homo; Human; 3TGF; Harvey et al., | 50 | 4.7 | 3.1 | 32/7 |
| Eur. J. Biochem. 198: 555 (1991)) | | | | |
| Metallothionein (Saccharomyces; Yeast; 1AOO; | 40 | 5.3 | 18.8 | 32/16 |
| Peterson et al., FEBS Lett. 379: 85 (1996)) | | | | |
| Allergen-5 (Ambrosia; Ragweed; 2BBG; | 40 | 6.5 | 18.8 | 32/7 |
| Metzler et al., Biochemistry 31: 5117 (1992)) | | | | |
| Ferredoxin (Clostridium; Bacteria; 2FDN; | 55 | 7.4 | 5.0 | 40/6 |
| Dauter et al., Biochemistry 36: 16065 (1997)) | | | | |

| | |
|---|---|
| Mean ± SD | 6.0 ± 1.2* |

Briefly, three-dimensional alignments of representative antimicrobial and control non-antimicrobial peptide structures were analyzed by pairwise comparison with Ah-AMP-1 (Aesculus; horsechestnut tree; 1BK8) using the combinatorial extension method (Shindyalov and Bourne, Protein Eng. 11:739 (1998)). Control peptides were selected from a cohort of 54 appropriate comparators based on disulfide content, sequence length, and molecular weight equivalence to Ah-AMP-1. Representative results are shown. The comparative length of each mature peptide is indicated as the number of amino acids (AAs). Root Mean Square Deviation (RMSD) values were determined for distances between α-carbon atoms over the length of the alignment. Percent identity is the percentage of sequence identity between the two peptides compared. The align/gap value indicates the number of residues considered for the alignment, and the number of gaps inserted. Relative gap penalties were integrated into the analysis. Mean RMSD values from antimicrobial versus non-antimicrobial peptides were significantly different (*) as determined by two tailed T-test (P<0.01). Information for each structure is formatted as follows: peptide name, (source genus; common name; Protein Data Bank [PDB] accession code; reference).

A highly conserved, disulfide-stabilized core motif was discovered to be integral to the 3-dimensional signature of cysteine-containing antimicrobial peptides. This feature is termed herein as the gamma-core motif (γ-core; FIG. 5). This structural motif is comprised of two anti-parallel β-sheets interposed by a short turn region. Notably, as shown in FIG. 4, the sequence patterns corresponding to the γ-core signature extends across the entire range of antimicrobial peptide families. Exemplary peptides included within the groups are: gomesin ([1KFP], Acanthoscurria, spider, (γ-Group); Mandard et al., Eur. J. Biochem. 269:1190 (2002)); protegrin-1 ([1PG1], Sus, domestic pig, (γ-Group)); thanatin ([8TFV], Podisus, soldier bug, (γ-Group)); α-defensin (HNP-3, [1DFN]; Homo, human, (β-γ-Group); β-defensin (MBD-8, [1E4R], Mus, mouse, (β-γ-Group)); fungal peptide (AFP-1, [1AFP], Aspergillus, fungus, (β-γ-α Group); insect-defensin (sapecin, [1L4V], Sarcophaga, flesh fly, (γ-α-Group)); crustacean

CS-αβ peptide (MGD-1, [1FJN], Mytilus, mussel, (γ-α-Group)); insect CS-αβ peptide (drosomycin, [1MYN], Drosophila, fruit fly, (γ-α-Group)); and plant CS-αβ□ peptide (Ah-AMP-1, [1BK8] Aesculus, horsechestnut tree, (β-γ-α Group)□. Other peptide data are formatted as in FIG. 3. See Table II for additional references. The conserved GXC (dextromeric) or CXG (levomeric) sequence patterns (FIG. 2B) are integrated into one β-sheet in this motif, reflecting conformational symmetry amongst antimicrobial peptides containing this signature (FIG. 5, respectively). Additional features that distinguish the γ-core include: 1) hydrophobic bias toward the C-terminal aspect; and 2) cationic charge positioned at the inflection point and termini of the β-sheet domains, polarizing charge along the longitudinal axis of the γ-core.

### Example II

### Validation of the Multidimensional Antimicrobial Peptide Signature Model

The multidimensional signature model for antimicrobial peptides integrates a stereospecific (dextromeric or levomeric) sequence pattern with the 3-dimensional gamma-core (“γ-core”). Therefore, this model predicted that peptides fulfilling these prerequisites would exert antimicrobial activity, even though such activity may not yet have been determined. Multiple and complementary approaches were used to test the model in this regard: 1) prediction of antimicrobial activity in peptides fulfilling the sequence and conformation criteria of the multidimensional signature, but not yet recognized to have antimicrobial activity;
2) predicted failure of antimicrobial activity in peptides exhibiting primary sequence criteria, but lacking the 3-dimensional □ γ-core signature of the model; and 3) prediction of a γ-core motif in disulfide-containing peptides with known antimicrobial activity, and which fulfilled primary sequence criteria, but had unknown structure.

To test the hypothesis that the primary sequence patterns of the multidimensional signature are relevant to all classes of disulfide-containing antimicrobial peptides, Swiss-Prot forward and reverse databases (Gattiker et al., Appl. Bioinformatics 1:107 (2002)) were queried with the enantiomeric sequence formulae. Representatives of all major disulfide-containing antimicrobial peptide classes were retrieved (Table III). Searches also retrieved members of other peptide subclasses: i) neurotoxins, particularly charybdotoxin class of the family Buthidae (scorpion); ii) protease inhibitor or related peptides (eg., brazzein) from plants; iii) ferredoxins; and iv) metallothioneins. Prototypes with known 3-dimensional structures, but no known antimicrobial activity, were analyzed for the presence of the γ-core signature. Of these, the peptides brazzein and charybdotoxin were selected to test for antimicrobial activity based on two criteria: i) their quantitative RMSD values reflected greatest homology to the comparator γ-core motif; and ii) they represented diverse non-mammalian (plant or scorpion) host sources and distinct structure classes not previously known to have antimicrobial activity. Thus, brazzein and charybdotoxin exemplified peptides that fulfilled the enantiomeric sequence and γ-core criteria required for the multidimensional signature. These peptides were predicted to have direct antimicrobial activity. In contrast, prototype metallothioneins and ferredoxins did not contain γ-core motifs (FIG. 3; Table II). Thus, metallothionein II was selected as an example comparator predicted to lack antimicrobial activity.

TABLE III
Recognition of diverse classes of antimicrobial peptides by the enantiomeric sequence formulae.

| | | Sequence Isoform | | | Proportion | |
|---|---|---|---|---|---|---|
| Antimicrobial Peptide Class | Phylogeny | Dextro | Levo-1 | Levo-2 | Total | % Total |
| α-defensin | Chordata | 24 | 42 | 6 | 72 | 15.3 |
| β-defensin | Chordata | 52 | 65 | 31 | 148 | 31.4 |
| θ-defensin | Chordata | 1 | 1 | 0 | 2 | 0.4 |
| Insect defensin/CS-αβ | Insectae | 21 | 23 | 12 | 56 | 11.9 |
| Plant defensin/CS-αβ | Plantae | 51 | 67 | 20 | 138 | 29.3 |
| Invertebrate defensin/CS-αβ | Mollusca | 3 | 4 | 4 | 11 | 2.3 |
| Protegrins/Gomesins | Chordata/Arthropoda | 0 | 0 | 6 | 6 | 1.3 |
| Tachyplesins/Polyphemusins | Arthropoda | 6 | 5 | 2 | 13 | 2.8 |
| Thanatin | Arthropoda | 0 | 1 | 0 | 1 | 0.2 |
| Mytilins/Big-Defensin | Mollusca | 3 | 3 | 2 | 8 | 1.7 |
| AFP-1 | Ascomycota | 1 | 0 | 0 | 1 | 0.2 |
| Lantibiotics/Microcins | Proteobacteria | 3 | 3 | 9 | 15 | 3.2 |
| | | 165 | 214 | 92 | 471 | |

Forward or reverse Swiss-Prot Databases (release 42.4; Nov. 14, 2003; 138,347 entries) were probed with formulae containing the dextromeric or levomeric motifs of the antimicrobial peptide signature using PROSITE (Gattiker et al., Appl. Bioinformatics 1: 107 (2002)). Data indicate the proportionate distribution of a non-redundant cohort of retrieval sets; in some cases, peptides were retrieved by more than one formula isoform. Note that search results include members of the lantibiotic superfamily of antimicrobial peptides that lack conventional disulfide bridges, but have alternate thioether stabilization.

These peptides were tested for antimicrobial activity against a panel of Gram-positive (Staphylococcus aureus, Bacillus subtilis) and Gram-negative (Escherichia coli) bacteria, and the fungus Candida albicans, using a well-established and sensitive in vitro assay [Antimicrobial activity was assessed using a well-established solid-phase diffusion method. Assays included well-characterized organisms: Staphylococcus aureus (ATCC 27217, Gram-positive); Bacillus subtilis (ATCC 6633, Gram-positive); Escherichia coli (strain ML-35, Gram-negative); and Candida albicans (ATCC 36082, fungus). In brief, organisms were cultured to logarithmic phase and inoculated at a density of 10⁶ colony forming units/ml in buffered molecular grade agarose at the indicated pH. Five μg of peptide resuspended in sterile deionized water were introduced into wells formed in the underlay, and incubated for 3 h at 37° C. Nutrient-containing overlay medium was then applied, and assays incubated at 37° C. or 30° C. for bacteria or fungi, respectively. Defensin HNP-1 was tested in parallel as a standard control. After 24 h, zones of complete or partial inhibition were measured. All assays were repeated independently a minimum of two times. Tang et al., Infect. Immun. 70:6524 (2002) for detailed methodology.].

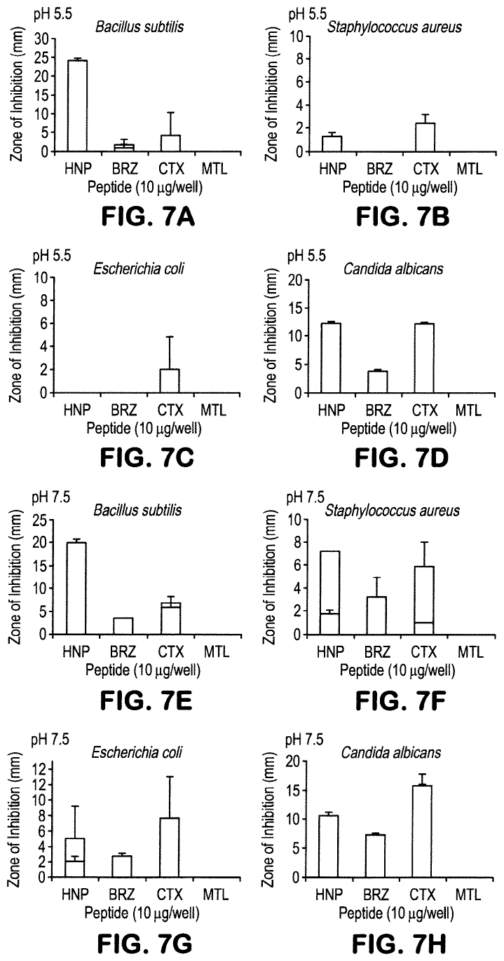

As predicted by the signature model, brazzein and charybdotoxin exerted direct antimicrobial activity against bacteria and C. albicans (FIG. 7). Notably, these peptides exhibited pH-specific antimicrobial activities, which in some conditions exceeded that of HNP-1. These results demonstrate for the first time to our knowledge the direct antimicrobial activities of brazzein and charybdotoxin. In contrast, metallothionein II failed to exert antimicrobial activity against any organism tested under any condition, as predicted by the model.

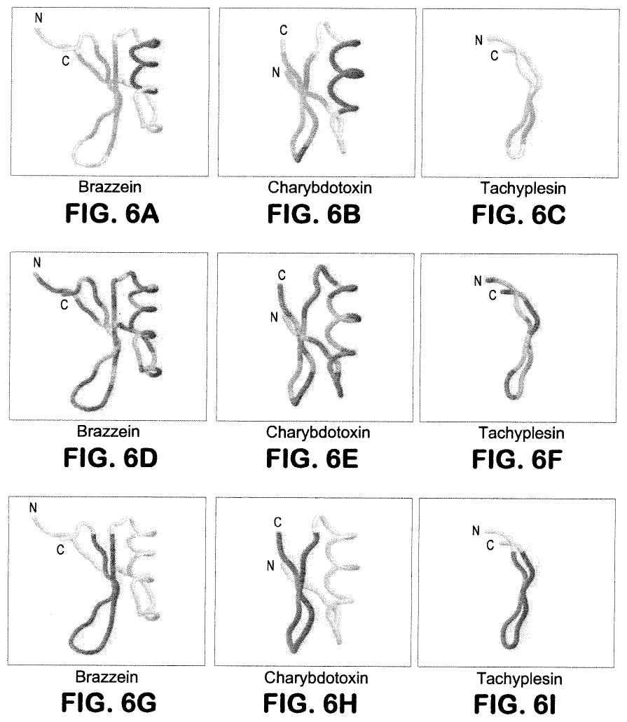

An alternative approach was also used to validate the multidimensional signature model. Tachyplesins are known cysteine-containing antimicrobial peptides from the horseshoe crab, Tachypleus. Two tachyplesins were retrieved from protein database searches employing the levomeric sequence formula (Table III). The model predicted that, because they have known antimicrobial activity, and fulfill the primary sequence criteria, tachyplesins would contain a γ-core motif. The 3-dimensional structure of tachyplesin I became available subsequent to development of the model (Laederach et al., Biochem. 41:12359 (2002)), and as predicted, exhibits a γ-core motif integral to the multidimensional signature of disulfide-containing antimicrobial peptides (FIG. 6). Confirmation of the 3-dimensional γ-core structure from antimicrobial activity and primary sequence pattern offers a robust and complementary validation of the multidimensional signature model.

The phylogenetic relationships among antimicrobial peptides containing the multidimensional signature were also examined. Study peptides sorted in a continuum of increasing structural complexity relative to the γ-core motif, rather than evolutionary relatedness of the source organisms (FIG. 8A). This phylogenetic pattern is consistent with conservation of the γ-core motif amongst cysteine-containing antimicrobial peptides across biological kingdoms.

### Example III

### Validation of the Multidimensional Antimicrobial Peptide Signature Model

This example demonstrates the discovery of iterations of the γ-core motif in kinocidins and validation of the antimicrobial signature model in human IL-8.

A bioinformatics approach was used to specify and compare phylogeny and homology among kinocidin iterations of the γ-core motif previously identified in proteins with known or predicted antimicrobial function (Yount, N. Y. & Yeaman, M. R. (2004) Proc Natl Acad Sci USA 101, 7363-8). The kinocidin γ-core (γ_{KC} core) signature is an iteration of the antimicrobial peptide γ-core (γ_{AP}), conforming to an anti-parallel β-hairpin comprised of a 13-17 amino acid pattern with a central hydrophobic region typically flanked by basic residues. The γ_{KC} core motif can be characterized by the following consensus sequence formula:

(SEQ ID NO: 363)
NH2 . . . [X8-11]-[GX3C]-[X2]-[P] . . . COOH

Sequence and 3-D analyses of more than 30 human CXC and CC kinocidins demonstrated that the γ_{KC} core corresponds to the most highly conserved domain within the mature portion of these proteins (FIG. 12). Notably, the cysteine array and glycine residues hallmark of the γ_{AP} core are also conserved in the γ_{KC} motif. In kinocidins, the GXC consensus of the γ_{AP} core is adapted such that a GX₃C pattern is often observed. While the initial glycine of the GX3C pattern of kinocidins is conserved (>60%), the requirement for a glycine in this position is not absolute, with uncharged hydrophilic residues (A or N) as most common substitutions. A proline residue at its C-terminal aspect is another highly conserved feature (>95%) of the γ_{KC} motif. This residue is located immediately prior to, and likely initiates the ensuing α-helical domain (FIG. 12).

Human IL-8 contains the sequence NH2 . . . CANTEIIVKLSDGRELCLDP . . . COOH (SEQ ID NO: 40), representing the γ_{KC} core consensus formula, and shares specific physicochemical patterns of amphipathicity, charge distribution, and proline positioning with known kinocidins (FIG. 12). Based on the extensive structural homologies to kinocidins, IL-8 was predicted to have direct antimicrobial efficacy.

To confirm that IL-8 exerts direct antimicrobial efficacy, antimicrobial assays using a panel of prototypic pathogens were conducted as described in the following paragraphs.

Briefly, the antimicrobial assays were performed against a panel of prototype human pathogens as previously detailed (Yoshimura, T., Matsushima, K., Tanaka, S., Robinson, E. A., Appella, E., Oppenheim, J. J. & Leonard, E. J. (1987) Proc Natl Acad Sci U S A 84, 9233-7): Staphylococcus aureus (ATCC 27217; Gram-positive bacterium); Salmonella typhimurium (5996s; Gram-negative bacterium); and the fungus Candida albicans (ATCC 36082). Defensin HNP-1 was included in each assay as an internal control, and all assays were conducted a minimum of two independent times. Results of independent assays were analyzed using Wilcoxon Rank Sum analysis with Bonferroni correction for multiple comparisons.

For the Solid-Phase Assay, mid-logarithmic phase organisms were prepared, introduced into buffered agarose (PIPES [10 mM, pH 7.5] or MES [2.0 mM, pH 5.5]) to achieve final inocula of 10⁶ CFU/ml, and poured into plates. Peptides (0.5 nmol/well [50 nmol/ml]) were added to wells in the seeded matrix, and incubated for 3 h at 37° C. Nutrient overlay medium was applied, and assays incubated at 37° C. or 30° C. for bacteria or fungi, respectively. After 24 h, zones of inhibition were measured, and results recorded as zones of complete or partial inhibition. This assay reflects microbiostatic (inhibition) and/or microbiocidal (killing) activities, but does not distinguish these effects.

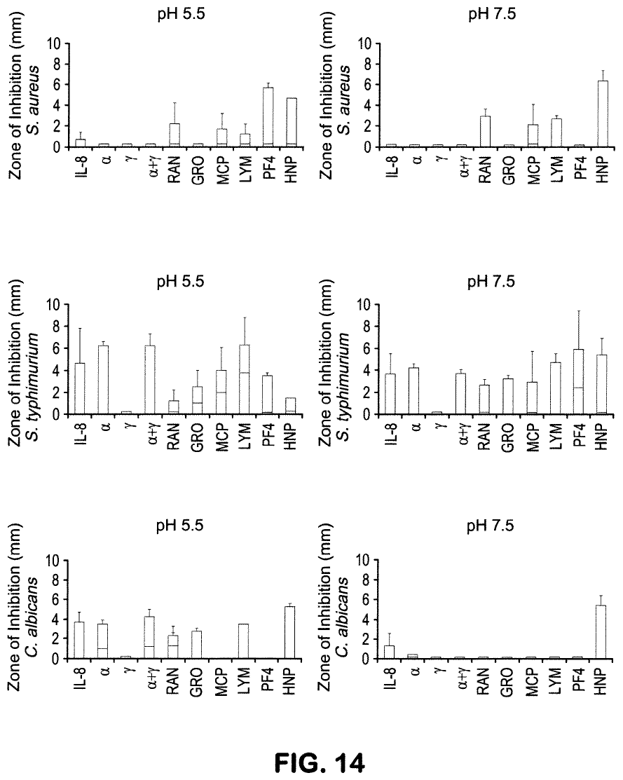

As predicted by biophysical and structural congruence with other kinocidins, IL-8 exerted antimicrobial activity against bacteria and fungi in the solid-phase assay at pH 5.5 and 7.5. In many cases, antimicrobial spectra and efficacy of IL-8 were greater than other study kinocidins (FIG. 14). IL-8 was equivalent to lymphotactin, but more efficacious than any other test kinocidin against S. typhimurium. Against S. aureus, IL-8 (pH 5.5) had modest activity, with RANTES (pH 7.5), and lymphotactin (pH 7.5) being more efficacious. Significantly, IL-8 possessed striking antifungal activity versus C. albicans, greater than any other kinocidin at pH 5.5, and the only kinocidin with anti-candidal activity at pH 7.5. Excepting S. aureus, IL-8 displayed comparable antimicrobial efficacy to the classical antimicrobial peptide HNP-1 under the conditions tested. In addition to IL-8, the these results also demonstrate a direct antimicrobial efficacy for the kinocidin MCP-1. Interestingly, while MCP-1 exerted significant activity against Gram-positive and-negative bacteria, it had no measurable antifungal activity (FIG. 14).

Recombinant human chemokines [IL-8, RANTES, GRO-α, MCP-1, PF-4, and lymphotactin (Biosource International, Camarillo, CA)] and human neutrophil defensin-1 [HNP-1 (Peptides International, Louisville, KY)] were obtained commercially. Structural domains of IL-8 were generated by F-moc solid-phase synthesis: γ-core (ANTEIIVKLSDGRELCLDP: IL-8γ (SEQ ID NO: 42)), and α-helix (KENWVQRVVEKFLKRAENS; IL-8α (SEQ ID NO: 1)). Peptides were purified by RP-HPLC as previously described ((Tang, Y. Q., Yeaman, M. R. & Selsted, M. E. (2002) Infect Immun 70, 6524-33); >95% purity), and authenticated by amino acid analysis (Molecular Structure Facility, University of California, Davis) and MALDI-TOF spectrometry (UCLA Spectrometry Facility). Experimentally determined masses were within standard confidence intervals (<±0.1% of calculated molecular weight).

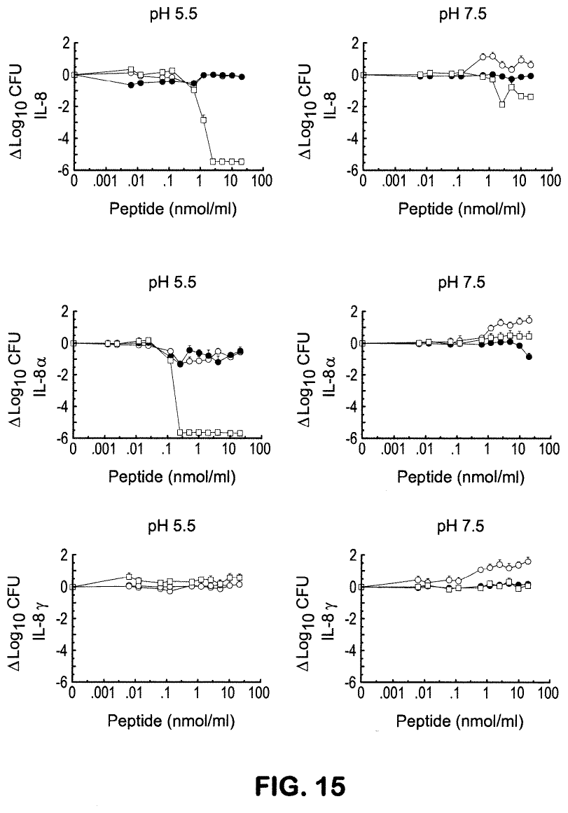

To characterize IL-8's antimicrobial activity in more detail, a quantitative solution-phase assay was carried out at pH 5.5 and 7.5. Organisms were prepared and adjusted to 10⁶ CFU/ml in PIPES or MES buffer as above but lacking agarose, and dispensed (5×10⁴ CFU per 50 μaliquot). Peptide (concentration range, 20-0.00125 nmol/ml) was introduced into the assay medium, and incubated for 1 hour at 37° C. After incubation, media were serially diluted and plated in triplicate for enumeration. IL-8 was highly fungicidal for C. albicans, achieving a five-log reduction in surviving CFU with 2.5 nmol/ml (20 μg/ml) peptide at pH 5.5 within 1 hour (FIG. 15). IL-8 was also fungicidal for C. albicans at pH 7.5, with a 2-log reduction in surviving CFU over 1 hour at a concentration of 2.5 nmol/ml. Interestingly, IL-8 did not exert microbicidal activity against S. typhimurium or S. aureus in this assay, indicating that its efficacies versus these organisms in solid-phase assay were due to bacteriostatic effects.

Synthetic IL-8γ and IL-8α peptides were used to probe for molecular determinants of IL-8antimicrobial activity. IL-8α displayed a spectrum of efficacy virtually indistinguishable from that of the native molecule (FIG. 14). IL-8γ had no detectable antimicrobial efficacy at either pH 5.5 or 7.5. When assayed in combination, the pattern of antimicrobial activity was identical to that of IL-8α alone, indicating that IL-8γ did not impede the antimicrobial efficacy of IL-8α.

In the solution-phase assay, as little as 0.125 nmol/ml (1.0 μg/ml) of IL-8α achieved a five-log reduction in surviving C. albicans at pH 5.5 in 1 hour exposure (FIG. 15). By mass, the autonomous IL-8α domain conferred a 10-fold greater activity than native IL-8 against this organism. The pH specific efficacy patterns of IL-8α also mirrored those of full-length (FIG. 15). Consistent with results of the solid-phase assay, IL-8γ had no measurable activity in the solution-phase assay.

To assess the physiological relevance of the observed in vitro (solid and solution-phase) antimicrobial activity for IL-8 and IL-8α, we assessed the efficacy of IL-8α in the ex vivo biomatrix assay. This assay has been developed to assess antimicrobial polypeptide efficacy in complex human blood matrices. Antimicrobial activity of IL-8α in human whole blood and homologous plasma and serum fractions was assessed (Yeaman, M. R., Gank, K. D., Bayer, A. S., and Brass, E. P. (2002) Antimicrob Agents Chemother 46, 3883-3891). For biomatrix studies, the well-characterized Escherichia coli strain ML-35 was used as the target organism (Lehrer, R. I., Barton, A., Daher, K. A., Harwig, S. S., Ganz, T., and Selsted, M. E. (1989) J Clin Invest 84, 553-561). This strain is resistant to serum, ideal for use in assessing peptide antimicrobial activity in blood and blood-derived matrices (Yeaman, M. R., Gank, K. D., Bayer, A. S., and Brass, E. P. (2002) Antimicrob Agents Chemother 46, 3883-3891). Organisms were cultured to mid-logarithmic phase in brain-heart infusion broth (Difco Laboratories, Detroit, Mich.) at 37° C., washed, and resuspended in PBS (Irvine Scientific; pH 7.2). Inocula were quantified spectrophotometrically and validated by quantitative culture. Biomatrices were distributed in 85-μl aliquots into 96-well microtiter plates (Corning Glass Works, Corning, N.Y.) Peptide (5 μl; concentration range 1.0-50.0 μg/ml) was added either simultaneously with the microorganism (10 μl; 10⁵ CFU/ml), or after a 120 min pre-incubation period in the biomatrix. The mixtures were incubated with constant agitation for 2 h at 37° C. After incubation, aliquots were diluted and quantitatively cultured in triplicate onto blood agar. Surviving organisms were enumerated as CFU/ml. Experiments were performed a minimum of two independent times on different days and with different blood donor sources.

Importantly, IL-8α demonstrated significant efficacy against E. coli, causing decreases of up to log 5 CFU/ml at 10 μg/ml peptide. Greatest efficacy was seen in whole blood and serum in co-incubation studies, with less activity in plasma fractions or after 2 hour preincubation.

To gain insights into these antimicrobial profiles, the structures of IL-8γ and IL-8α were investigated using biophysical and computational methods.

Secondary structures of IL-8 peptide domains were assessed by circular dichroism (CD) as previously noted (Sheppard et al. (2004) J Biol Chem 279, 30480-30489). Spectra were recorded with an AVIV 62DS spectropolarimeter (Aviv Biomedical Inc.). In brief, the purified peptides were solubilized (50 μg/ml in 50 mM NH4HC03; pH 5.5 or 7.5) and scanned using a 0.1-mm light path from 260 to 185 nm (rate, 10 nm/min; sample interval, 0.2 nm; 25° C.). A mean of 8 buffer-subtracted spectra were deconvoluted into helix, β-sheet, turn, and extended structures using Selcon (Sreerama et al. (1999) Protein Sci 8, 370-380) and Dichroweb (Lobley et al., (2002) Bioinformatics 18, 211-212); cryst.bbk.ac.uk/cdweb) as indicated (Sheppard et al. (2004) J Biol Chem 279, 30480-30489; Surewicz and Mantsch (1988) Biochim Biophys Acta 952, 115-130; Goormaghtighet al. (1999) Biochim Biophys Acta 1422, 105-185).

Protein structure data were obtained from the National Center for Biotechnology Information (NCBI; PubMed Database), and visualized using Protein Explorer (Martz, E. (2002) Trends Biochem Sci 27, 107-109). Structural alignments were carried out using combinatorial extension, and significance of homology assessed by root mean square deviation analysis, as previously described (Yount and Yeaman (2004) Proc Natl Acad Sci U S A 101, 7363-7368; Shindyalov and Bourne (1998) Protein Eng 11, 739-747).

3CD spectrometry indicated that IL-8γ and IL-8α recapitulated structures of corresponding regions in full-length IL-8 (FIG. 16). IL-8γ exhibited spectra consistent with a β-sheet structure, suggesting it spontaneously adopts a fold similar to that in native IL-8. Likewise, IL-8α displayed classic double dichroic minima at 208 and 218 nm, hallmark of α-helices, and concordant with the corresponding region in IL-8. These data suggest the forces responsible for secondary structures of these domains function independently from cysteine-stabilization or other constraints acting within the native molecule. Moreover, each structure was stable at pH 5.5 and pH 7.2 (FIG. 16).

To complement spectrometric studies, 3-D models of IL-8γ and IL-8α were created using homology and energy-based methods. Three-dimensional models of IL-8 domains were created using complementary methods (Yount et al. (2004) Antimicrob Agents Chemother 48, 4395-404.). Homology [SWISSMODEL, BLAST2P; (Godzik et al. (1992) J Mol Biol 227, 227-38; Jaroszewski et al. (1998) Protein Sci 7, 1431-40], dynamic alignment (SIM) (Huang, X., and Miller, W. (1991) Adv. Appl. Math. 12, 337-367) and refined match (ProModII) algorithms were used to identify modeling templates. In a parallel strategy, IL-8 amino acid sequences were converted to putative solution conformations by threading methods (Matchmaker [Godzik et al. (1992) J Mol Biol 227, 227-238]; Gene-Fold [Jaroszewski et al. (1998) Protein Sci 7, 1431-1440]) implemented with SYBYL software (Tripos Associates, St. Louis, Mo). Target conformers were refined using AMBER95 force field and molecular dynamics (Cornell et al. (1995) J Am Chem Soc 117, 5179-5197). In alternative approaches, molecular dynamics were executed without 0.4 kJ constraint penalties for canonical Ramachandran φ and ψ angles.

The template utilized for target peptide modeling was human IL-8 (PDB code, 1IL8). As expected, each peptide retained secondary structure corresponding to homologous domains within the native molecule (FIG. 17). The IL-8β core motif displayed an anti-parallel β-sheet motif, while the preferential conformation for IL-8α was a highly stable α-helical motif comprised of four turns. These structure assignments are strongly supported by favorable empirical energy functions, equivalent to those of the IL-8 template.

TABLE III
Comparative physicochemical properties of human
kinocidins and α-helical domains thereof.

| Classification Schema | | | Native Molecule | | | | α-Helical Domain | | | | |
|---|---|---|---|---|---|---|---|---|---|---|---|
| Class | Ligand ID | Name | AA | M | Q | pI | AA_{α} | Q_{α} | M_{α} | pI_{α} | H_{α} |
| CXC | CXCL8 | IL-8 | 71 | 8299 | +4 | 9.0 | 17 | +2 | 2103 | 10.0 | 6.70 |
| CXC | CXCL4 | PF-4 | 70 | 7769 | +3 | 8.8 | 13 | +3 | 1573 | 9.8 | 6.12 |
| CXC | CXCL1 | GRO-α | 72 | 7751 | +6 | 9.5 | 16 | +2 | 1843 | 9.6 | 4.71 |
| CC | CCL2 | MCP-1 | 76 | 8685 | +6 | 9.4 | 19 | 0 | 2287 | 6.8 | 5.35 |
| CC | CCL5 | RANTES | 68 | 7851 | +5 | 9.2 | 13 | 0 | 1655 | 6.1 | 6.71 |
| C | CL1 | Lymphotactin | 92 | 10173 | +9 | 10.6 | 14 | +2 | 1735 | 10.7 | 3.73 |

Physicochemical parameters are abbreviated for the native or α-helical domain (α),:
AA-amino acids;
M-average mass (Da);
Q-calculated charge at pH 7.0;
pI-estimated isoelectric point (Bjellqvist et al., (1994) Electrophoresis 15, 529-39);
H-hydrophobic moment (Zidovetzki et al. (2003) Biophys Chem 100, 555-75).

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A kinocidin peptide comprising a C-terminal portion amino acid sequence of a kinocidin, wherein said C-terminal portion comprises an α-helical secondary structure, wherein said C-terminal portion further comprises antimicrobial activity, and wherein said kinocidin comprises a YKC core.

2. The kinocidin peptide of claim 1, wherein the kinocidin is a CXC, CX3C, CC, or C class chemokine.

3. The kinocidin peptide of claim 1, wherein said amino acid sequence is KENWVQRVVEKFLKRAENS (SEQ ID NO: 1).

4. The kinocidin peptide of claim 1, wherein said amino acid sequence is QAPLYKKIIKKLLES (SEQ ID NO: 2).

5. The kinocidin peptide of claim 1, wherein said amino acid sequence is ASPIVKKIIEKMLNSDKSN (SEQ ID NO: 3).

6. The kinocidin peptide of claim 1, wherein said amino acid sequence is selected from the group depicted in FIG. 21.

7. The kinocidin peptide of claim 1, wherein said alpha-helical secondary structure comprises between 10 and 35 amino acids.

8. The kinocidin peptide of claim 1, wherein said alpha-helical secondary structure comprises a mass between 1100 Da and 3850 Da.

9. The kinocidin peptide of claim 1, wherein said alpha-helical secondary structure comprises a calculated charge between 0 and (+) 5 at pH 7.0.

10. The kinocidin peptide of 1, wherein said alpha-helical secondary structure comprises an estimated isoelectric point between 5 and 15.

11. The kinocidin peptide of claim 1, wherein said alpha-helical secondary structure comprises a hydrophobic moment between 3 and 8.

12. A method for treating an infectious disease or condition in a subject in need of such treatment comprising administering to the subject an effective amount of a kinocidin peptide of claims 1 through 11.


SEQUENCE LISTING (361 sequences)

SEQ ID NO: 1 - protein, 19 aa, synthetic construct
  REGION 1..19: note = Description of Artificial Sequence: Synthetic peptide
KENWVQRVVEKFLKRAENS

SEQ ID NO: 2 - protein, 15 aa, synthetic construct
  REGION 1..15: note = Description of Artificial Sequence: Synthetic peptide
QAPLYKKIIKKLLES

SEQ ID NO: 3 - protein, 19 aa, synthetic construct
  REGION 1..19: note = Description of Artificial Sequence: Synthetic peptide
ASPIVKKIIEKMLNSDKSN

SEQ ID NO: 4 - protein, 18 aa, synthetic construct
  REGION 1..18: note = Description of Artificial Sequence: Synthetic peptide
DAPRIKKIVQKKLAGDES

SEQ ID NO: 5 - protein, 19 aa, synthetic construct
  REGION 1..19: note = Description of Artificial Sequence: Synthetic peptide
ASPMVKKIIEKMLKNGKSN

SEQ ID NO: 6 - protein, 19 aa, synthetic construct
  REGION 1..19: note = Description of Artificial Sequence: Synthetic peptide
ASPMVQKIIEKILNKGSTN

SEQ ID NO: 7 - protein, 20 aa, synthetic construct
  REGION 1..20: note = Description of Artificial Sequence: Synthetic peptide
EAPFLKKVIQKILDGGNKEN

SEQ ID NO: 8 - protein, 20 aa, synthetic construct
  REGION 1..20: note = Description of Artificial Sequence: Synthetic peptide
EAPFLKKVIQKILDSGNKKN

SEQ ID NO: 9 - protein, 20 aa, synthetic construct
  REGION 1..20: note = Description of Artificial Sequence: Synthetic peptide
DAPRIKKIVQKKLAGDESAD

SEQ ID NO: 10 - protein, 21 aa, synthetic construct
  REGION 1..21: note = Description of Artificial Sequence: Synthetic peptide
ESKAIKNLLKAVSKERSKRSP

SEQ ID NO: 11 - protein, 17 aa, synthetic construct
  REGION 1..17: note = Description of Artificial Sequence: Synthetic peptide
KSKQARLIIKKVERKNF

SEQ ID NO: 12 - protein, 19 aa, synthetic construct
  REGION 1..19: note = Description of Artificial Sequence: Synthetic peptide
KLKWIQEYLEKALNKRFKM

SEQ ID NO: 13 - protein, 30 aa, synthetic construct
  REGION 1..30: note = Description of Artificial Sequence: Synthetic peptide
QAEWIQRMMEVLRKRSSSTLPVPVFKRKIP

SEQ ID NO: 14 - protein, 24 aa, synthetic construct
  REGION 1..24: note = Description of Artificial Sequence: Synthetic peptide
KLQSTKRFIKWYNAWNEKRRVYEE

SEQ ID NO: 15 - protein, 20 aa, synthetic construct
  REGION 1..20: note = Description of Artificial Sequence: Synthetic peptide
TVGWVQRHRKMLRHCPSKRK

SEQ ID NO: 16 - protein, 21 aa, synthetic construct
  REGION 1..21: note = Description of Artificial Sequence: Synthetic peptide
KQKWVQDSMDHLDKQTQTPKT

SEQ ID NO: 17 - protein, 16 aa, synthetic construct
  REGION 1..16: note = Description of Artificial Sequence: Synthetic peptide
SEEWVQKYVSDLELSA

SEQ ID NO: 18 - protein, 15 aa, synthetic construct
  REGION 1..15: note = Description of Artificial Sequence: Synthetic peptide
SESWVQEYVYDLELN

SEQ ID NO: 19 - protein, 15 aa, synthetic construct
  REGION 1..15: note = Description of Artificial Sequence: Synthetic peptide
EKKWVREYINSLEMS

SEQ ID NO: 20 - protein, 21 aa, synthetic construct
  REGION 1..21: note = Description of Artificial Sequence: Synthetic peptide
TQKWVQDFMKHLDKKTQTPKL

SEQ ID NO: 21 - protein, 21 aa, synthetic construct
  REGION 1..21: note = Description of Artificial Sequence: Synthetic peptide
KERWVRDSMKHLDQIFQNLKP

SEQ ID NO: 22 - protein, 21 aa, synthetic construct
  REGION 1..21: note = Description of Artificial Sequence: Synthetic peptide
KKKWVQDSMKYLDQKSPTPKP

SEQ ID NO: 23 - protein, 21 aa, synthetic construct
  REGION 1..21: note = Description of Artificial Sequence: Synthetic peptide
KEKWVQNYMKHLGRKAHTLKT

SEQ ID NO: 24 - protein, 15 aa, synthetic construct
  REGION 1..15: note = Description of Artificial Sequence: Synthetic peptide
SDKWVQDYIKDMKEN

SEQ ID NO: 25 - protein, 17 aa, synthetic construct
  REGION 1..17: note = Description of Artificial Sequence: Synthetic peptide
SGPGVQDCMKKLKPYSI

SEQ ID NO: 26 - protein, 28 aa, synthetic construct
  REGION 1..28: note = Description of Artificial Sequence: Synthetic peptide
NDDWVQEYIKDPNLPLLPTRNLSTVKII

SEQ ID NO: 27 - protein, 18 aa, synthetic construct
  REGION 1..18: note = Description of Artificial Sequence: Synthetic peptide
NNKRVKNAVKYLQSLERS

SEQ ID NO: 28 - protein, 16 aa, synthetic construct
  REGION 1..16: note = Description of Artificial Sequence: Synthetic peptide
NKKWVQKYISDLKLNA

SEQ ID NO: 29 - protein, 24 aa, synthetic construct
  REGION 1..24: note = Description of Artificial Sequence: Synthetic peptide
DQPWVERIIQRLQRTSAKMKRRSS

SEQ ID NO: 30 - protein, 19 aa, synthetic construct
  REGION 1..19: note = Description of Artificial Sequence: Synthetic peptide
KQTWVKYIVRLLSKKVKNM

SEQ ID NO: 31 - protein, 24 aa, synthetic construct
  REGION 1..24: note = Description of Artificial Sequence: Synthetic peptide
KELWVQQLMQHLDKTPSPQKPAQG

SEQ ID NO: 32 - protein, 14 aa, synthetic construct
  REGION 1..14: note = Description of Artificial Sequence: Synthetic peptide
RVPWVKMILNKLSQ

SEQ ID NO: 33 - protein, 23 aa, synthetic construct
  REGION 1..23: note = Description of Artificial Sequence: Synthetic peptide
SDKQVQVCVRMLKLDTRIKTRKN

SEQ ID NO: 34 - protein, 24 aa, synthetic construct
  REGION 1..24: note = Description of Artificial Sequence: Synthetic peptide
KQEWVQRYMKNLDAKQKKASPRAR

SEQ ID NO: 35 - protein, 17 aa, synthetic construct
  REGION 1..17: note = Description of Artificial Sequence: Synthetic peptide
KSREVQRAMKLLDARNK

SEQ ID NO: 36 - protein, 32 aa, synthetic construct
  REGION 1..32: note = Description of Artificial Sequence: Synthetic peptide
QNPSLSQWFEHQERKLHGTLPKLNFGMLRKMG

SEQ ID NO: 37 - protein, 21 aa, synthetic construct
  REGION 1..21: note = Description of Artificial Sequence: Synthetic peptide
HNHTVKQWMKVQAAKKNGKGN

SEQ ID NO: 38 - protein, 21 aa, synthetic construct
  REGION 1..21: note = Description of Artificial Sequence: Synthetic peptide
QATWVRDVVRSMDRKSNTRNN

SEQ ID NO: 39 - protein, 22 aa, synthetic construct
  REGION 1..22: note = Description of Artificial Sequence: Synthetic peptide
  VARIANT 2..14: note = region may encompass between 10 and 13 variable amino acids
  VARIANT 16..18: note = region may encompass between 2 and 3 variable amino acids
  VARIANT 20..21: note = variable amino acid
CXXXXXXXXXXXXXGXXXCXXP

SEQ ID NO: 40 - protein, 20 aa, synthetic construct
  REGION 1..20: note = Description of Artificial Sequence: Synthetic peptide
CANTEIIVKLSDGRELCLDP

SEQ ID NO: 41 - protein, 22 aa, synthetic construct
  REGION 1..22: note = Description of Artificial Sequence: Synthetic peptide
  VARIANT 2..5: note = variable amino acid
  VARIANT 6..8: note = variable hydrophobic amino acid residue
  VARIANT 9..10: note = region may encompass between 0 and 2 variable amino acids
  VARIANT 12..14: note = region may encompass between 1 and 3 variable amino acids
  VARIANT 16: note = Lys or Arg
  VARIANT 17: note = variable charged or polar amino acid residue
  VARIANT 18: note = variable hydrophobic amino acid residue
  VARIANT 20: note = variable hydrophobic amino acid residue
  VARIANT 21: note = Asp or Asn
CXXXXXXXXXKXXXGXXXCXXP

SEQ ID NO: 42 - protein, 19 aa, synthetic construct
  REGION 1..19: note = Description of Artificial Sequence: Synthetic peptide
ANTEIIVKLSDGRELCLDP

SEQ ID NO: 43 - protein, 19 aa, unidentified
  VARIANT 19: note = variable amino acid
  source 1..19: note = Sus sp.
RGGRLCYCRRRFCVCVGRX

SEQ ID NO: 44 - protein, 19 aa, unidentified
  VARIANT 1: note = variable amino acid
  VARIANT 19: note = variable amino acid
  source 1..19: note = Acanthoscurria sp.
XCRRLCYKQRCVTYCRGRX

SEQ ID NO: 45 - protein, 44 aa, unidentified
  source 1..44: note = Drosophila sp.
DCLSGRYKGPCAVWDNETCRRVCKEEGRSSGHCSPSLKCWCEGC

SEQ ID NO: 46 - protein, 39 aa, Mytilus sp.
GFGCPNNYQCHRHCKSIPGRCGGYCGGWHRLRCTCYRCG

SEQ ID NO: 47 - protein, 17 aa, unidentified
  source 1..17: note = Tachypleus sp.
KWCFRVCYRGICYRRCR

SEQ ID NO: 48 - protein, 34 aa, unidentified
  source 1..34: note = Mytilus sp.
GCASRCKAKCAGRRCKGWASASFRGRCYCKCFRC

SEQ ID NO: 49 - protein, 40 aa, unidentified
  source 1..40: note = Protophormia sp.
ATCDLLSGTGINHSACAAHCLLRGNRGGYCNGKAVCVCRN

SEQ ID NO: 50 - protein, 30 aa, Homo sapiens
DCYCRIPACIAGERRYGTCIYQGRLWAFCC

SEQ ID NO: 51 - protein, 50 aa, unidentified
  source 1..50: note = Aesculus sp.
LCNERPSQTWSGNCGNTAHCDKQCQDWEKASHGACHKRENHWKCFCYFNC

SEQ ID NO: 52 - protein, 50 aa, unidentified
TYNGKCYKKDNICKYKAQSGKTAICKCYVKKCPRDGAKCEFDSYKGKCYC

SEQ ID NO: 53 - protein, 35 aa, unidentified
  VARIANT 1: note = variable amino acid
  source 1..35: note = Mus sp.
XEPVSCIRNGGICQYRCIGLRHKIGTCGSPFKCCK

SEQ ID NO: 54 - protein, 21 aa, unidentified
  source 1..21: note = Podisus sp.
GSKKPVPIIYCNRRTGKCQRM

SEQ ID NO: 55 - protein, 33 aa, unidentified
  source 1..33: note = Rana sp.
SLFSLIKAGAKFLGKNLLKQGACYAACKASKQC

SEQ ID NO: 56 - protein, 30 aa, Homo sapiens; identical to SEQ ID NO: 50

SEQ ID NO: 57 - protein, 34 aa, unidentified
  source 1..34: note = Mus sp.
EPVSCIRNGGICQYRCIGLRHKIGTCGSPFKCCK

SEQ ID NO: 58 - protein, 40 aa, Protophormia sp.
ATCDLLSGTGINHSACAAHCLLRGNRGGYCNGKGVCVCRN

SEQ ID NO: 59 - protein, 44 aa, Drosophila sp.; identical to SEQ ID NO: 45

SEQ ID NO: 60 - protein, 49 aa, Aesculus sp.
CNERPSQTWSGNCGNTAHCDKQCQDWEKASHGACHKRENHWKCFCYFNC

SEQ ID NO: 61 - protein, 39 aa, Mytilus sp.; identical to SEQ ID NO: 46

SEQ ID NO: 62 - protein, 18 aa, Sus sp.
RGVCVCFRRRCYCLRGGR

SEQ ID NO: 63 - protein, 34 aa, Mytilus sp.
CRFCKCYCRGRFSASAWGKCRRGACKAKCRSACG

SEQ ID NO: 64 - protein, 33 aa, Rana sp.; identical to SEQ ID NO: 55

SEQ ID NO: 65 - protein, 17 aa, Acanthoscurria sp.
RGRCYTVCRQKYCLRRC

SEQ ID NO: 66 - protein, 21 aa, Podisus sp.
MRQCKGTRRNCYIIPVPKKSG

SEQ ID NO: 67 - protein, 17 aa, Tachypleus sp.
RCRRYCIGRYCVRFCWK

SEQ ID NO: 68 - protein, 25 aa, unidentified
  source 1..25: note = Aspergillus sp.
CYCKGKYSDFECKAGDRPCKKVYCK

SEQ ID NO: 69 - protein, 18 aa, Sus sp.
RGVCVCFRRRCYCLRGGR

SEQ ID NO: 70 - protein, 17 aa, Acanthoscurria sp.
RGRCYTVCRQKYCLRRC

SEQ ID NO: 71 - protein, 17 aa, Tachypleus sp.
RCRRYCIGRYCVRFCWK

SEQ ID NO: 72 - protein, 14 aa, Macaca sp.
CICRCVGRRCLCRC

SEQ ID NO: 73 - protein, 12 aa, Podisus sp.
RQCKGTRRNCYI

SEQ ID NO: 74 - protein, 17 aa, Homo sapiens
CIFCCGCCHRSKCGMCC

SEQ ID NO: 75 - protein, 15 aa, Sarcophaga sp.
RGGYCNGKAVCVCRN

SEQ ID NO: 76 - protein, 15 aa, Protophormia sp.
RGGYCNGKGVCVCRN

SEQ ID NO: 77 - protein, 17 aa, Heliothis sp.
KGGHCGSFANVNCWCET

SEQ ID NO: 78 - protein, 16 aa, Drosophila sp.
SSGHCSPSLKCWCEGC

SEQ ID NO: 79 - protein, 20 aa, Mytilus sp.
RCGGYCGGWHRLRCTCYRCG

SEQ ID NO: 80 - protein, 14 aa, unidentified
  source 1..14: note = Leiurus sp.
SRGKCMNKKCRCYS

SEQ ID NO: 81 - protein, 16 aa, Homo sapiens
RYGTCIYQGRLWAFCC

SEQ ID NO: 82 - protein, 20 aa, Oryctolagus sp.
EVIDGSCGLFNSKYICCREK

SEQ ID NO: 83 - protein, 18 aa, Bos sp.
MRQIGTCFGRPVKCCRSW

SEQ ID NO: 84 - protein, 15 aa, Homo sapiens
KIQGTCYRGKAKCCK

SEQ ID NO: 85 - protein, 19 aa, Homo sapiens
RYKQIGTCGLPGTKCCKKP

SEQ ID NO: 86 - protein, 14 aa, Mus sp.
HKIGTCGSPFKCCK

SEQ ID NO: 87 - protein, 20 aa, Aesculus sp.
SHGACHKRENHWKCFCYFNC

SEQ ID NO: 88 - protein, 20 aa, Raphanus sp.
RHGSCNYVFPAHKCICYFPC

SEQ ID NO: 89 - protein, 16 aa, Pisum sp.
ISGTCHNWKCFCTQNC

SEQ ID NO: 90 - protein, 18 aa, Hordeum sp.
GGGNCDGPLRRCKCMRRC

SEQ ID NO: 91 - protein, 18 aa, Triticum sp.
GGGNCDGPFRRCKCIRQC

SEQ ID NO: 92 - protein, 19 aa, unidentified
  source 1..19: note = Pentadiplandra sp.
RSGECFYDEKRNLQCICDY

SEQ ID NO: 93 - protein, 23 aa, synthetic construct
  REGION 1..23: note = Description of Artificial Sequence: Synthetic consensus peptide
  VARIANT 1..3: note = variable amino acid
  VARIANT 5: note = variable amino acid
  VARIANT 7..15: note = variable amino acid
  VARIANT 18..23: note = variable amino acid
XXXGXCXXXXXXXXXCCXXXXXX

SEQ ID NO: 94 - protein, 18 aa, Rattus sp.
RLSGACGYRGRIYRLCCR

SEQ ID NO: 95 - protein, 18 aa, Rattus sp.
RLSGACGYRGRIYRLCCR

SEQ ID NO: 96 - protein, 17 aa, Rattus sp.
RLSGACRLNGRIYRLCC

SEQ ID NO: 97 - protein, 17 aa, Rattus sp.
RLSGACRLNGRIYRLCC

SEQ ID NO: 98 - protein, 18 aa, Rattus sp.
RLTGACGLNGRIYRLCCR

SEQ ID NO: 99 - protein, 18 aa, Homo sapiens
SLSGVCEISGRLYRLCCR

SEQ ID NO: 100 - protein, 19 aa, Oryctolagus sp.
RRAGFCRIRGRIHPLCCRR

SEQ ID NO: 101 - protein, 19 aa, Oryctolagus sp.
RRAGFCRIRGRIHPLCCRR

SEQ ID NO: 102 - protein, 20 aa, Oryctolagus sp.
RFSGYCRVNGARYVRCCSRR

SEQ ID NO: 103 - protein, 20 aa, Oryctolagus sp.
QFSGYCRVNGARYVRCCSRR

SEQ ID NO: 104 - protein, 19 aa, Oryctolagus sp.
RASGSCTVNGVRHTLCCRR

SEQ ID NO: 105 - protein, 19 aa, Oryctolagus sp.
RASGSCTINGVRHTLCCRR

SEQ ID NO: 106 - protein, 17 aa, Cavia sp.
RRLGTCIFQNRVYTFCC

SEQ ID NO: 107 - protein, 17 aa, Cavia sp.
RRLGTCIFQNRVYTFCC

SEQ ID NO: 108 - protein, 17 aa, Macaca sp.
RRYGTCFYMGRVWAFCC

SEQ ID NO: 109 - protein, 17 aa, Homo sapiens
RRYGTCIYQGRLWAFCC

SEQ ID NO: 110 - protein, 21 aa, Homo sapiens
LRVGNCLIGGVSFTYCCTRVD

SEQ ID NO: 111 - protein, 19 aa, Mesocricetus auratus
RHIGYCRFGNTIYRLCCRR

SEQ ID NO: 112 - protein, 19 aa, Mesocricetus auratus
RLIGYCRFGNTIYGLCCRR

SEQ ID NO: 113 - protein, 19 aa, Mesocricetus auratus
TQIGYCRLGNTFYRLCCRQ

SEQ ID NO: 114 - protein, 19 aa, Mesocricetus auratus
TQIGYCRLGNTFYRLCCRQ

SEQ ID NO: 115 - protein, 18 aa, Mus sp.
RMNGTCRKGHLLYTLCCR

SEQ ID NO: 116 - protein, 18 aa, Mus sp.
RMNGTCRKGHLLYMLCCR

SEQ ID NO: 117 - protein, 18 aa, Mus sp.
RMNGTCRKGHLMYTLCCR

SEQ ID NO: 118 - protein, 18 aa, Mus sp.
RMNGTCRKGHLLYMLCCR

SEQ ID NO: 119 - protein, 18 aa, Mus sp.
RMNGTCRKGHLLYTMCCR

SEQ ID NO: 120 - protein, 18 aa, Mus sp.
RMNGTCRKGHLMYTLCCR

SEQ ID NO: 121 - protein, 18 aa, Mus sp.
RMNGTCRKGHLMHTLCCR

SEQ ID NO: 122 - protein, 18 aa, Mus sp.
HMNGTCRKGHLMYTLCCR

SEQ ID NO: 123 - protein, 18 aa, Mus sp.
HMNGTCRRGHLMYTLCCR

SEQ ID NO: 124 - protein, 18 aa, Mus sp.
HMNGTCRKGHLMYTLCCR

SEQ ID NO: 125 - protein, 18 aa, Mus sp.
RMNGTCRKGHLMYTLCCR

SEQ ID NO: 126 - protein, 18 aa, Mus sp.
HMNGTCRKGHLLYMLCCR

SEQ ID NO: 127 - protein, 18 aa, Mus sp.
HINGTCRKGHLLYMLCCR

SEQ ID NO: 128 - protein, 18 aa, Mus sp.
HMNGTCRKGHLLYTLCCR

SEQ ID NO: 129 - protein, 17 aa, Mus sp.
RVRGTCGIRFLYCCPRR

SEQ ID NO: 130 - protein, 20 aa, Mus sp.
RVFGTCRNLFLTFVFCCSRR

SEQ ID NO: 131 - protein, 18 aa, Rattus sp.
GIMGICKKRYGSPILCCR

SEQ ID NO: 132 - protein, 14 aa, Mus sp.
HKIGTCGSPFKCCR

SEQ ID NO: 133 - protein, 17 aa, Bos sp.
IQIGICFRPRVKCCRSW

SEQ ID NO: 134 - protein, 15 aa, Bos sp.
RQIGTCLAPRVKCCR

SEQ ID NO: 135 - protein, 17 aa, Capra hircus
RQIGTCFGPPVKCCRKK

SEQ ID NO: 136 - protein, 17 aa, Ovis sp.
RQIGTCFGPPVKCCRLK

SEQ ID NO: 137 - protein, 17 aa, Bos sp.
RQIGTCFGRPVKCCRSW

SEQ ID NO: 138 - protein, 17 aa, Bos sp.
RQIGTCFGRPVKCCRSW

SEQ ID NO: 139 - protein, 17 aa, Bos sp.
RQIGTCFGRPVKCCRSW

SEQ ID NO: 140 - protein, 17 aa, Bos sp.
RQIGTCFGRPVKCCRSW

SEQ ID NO: 141 - protein, 17 aa, Ovis sp.
RQIGTCRGPPVKCCRKK

SEQ ID NO: 142 - protein, 17 aa, Bos sp.
RQIGTCFGPRIKCCRSW

SEQ ID NO: 143 - protein, 17 aa, Bos sp.
RQIGTCFGPRIKCCRSW

SEQ ID NO: 144 - protein, 17 aa, Bos sp.
RQIGTCFGRPVKCCRRW

SEQ ID NO: 145 - protein, 15 aa, Bos sp.
RQIGTCLGPQIKCCR

SEQ ID NO: 146 - protein, 15 aa, Bos sp.
RQIGTCLAPQIKCCR

SEQ ID NO: 147 - protein, 15 aa, Bos sp.
RQIGTCLGPRIKCCR

SEQ ID NO: 148 - protein, 17 aa, Bos sp.
RQIGTCLGAQVKCCRRK

SEQ ID NO: 149 - protein, 17 aa, Bos sp.
KQIGTCVGRAVKCCRKK

SEQ ID NO: 150 - protein, 17 aa, Bos sp.
RQIGTCFTPSVKCCRWR

SEQ ID NO: 151 - protein, 16 aa, Bos sp.
RQIGTCFGPRVPCCRR

SEQ ID NO: 152 - protein, 17 aa, Bos sp.
RQIGTCFGPRVPCCRRW

SEQ ID NO: 153 - protein, 17 aa, Sus sp.
KQIGTCGMPQVKCCKRK

SEQ ID NO: 154 - protein, 17 aa, Homo sapiens
KQIGTCGLPGTKCCKKP

SEQ ID NO: 155 - protein, 17 aa, Rattus sp.
RQIGTCGLPRVRCCKKK

SEQ ID NO: 156 - protein, 17 aa, Mus sp.
RQIGNCGHFKVRCCKIR

SEQ ID NO: 157 - protein, 17 aa, Mus sp.
RQIGSCGVFPLKCCKRK

SEQ ID NO: 158 - protein, 15 aa, Homo sapiens
KIQGTCYRGKAKCCK

SEQ ID NO: 159 - protein, 15 aa, Macaca mulatta
RIQGTCYHGKAKCCK

SEQ ID NO: 160 - protein, 16 aa, Mus sp.
KLQGTCKPDKPNCCKS

SEQ ID NO: 161 - protein, 16 aa, Rattus sp.
KLQGTCKPDKPNCCRS

SEQ ID NO: 162 - protein, 18 aa, Mus sp.
RRPGSCFPEKNPCCKYMK

SEQ ID NO: 163 - protein, 18 aa, Homo sapiens
EQIGKCSTRGRKCCRRKK

SEQ ID NO: 164 - protein, 17 aa, Gallus sp.
LISGKCSRFYLCCKRIR

SEQ ID NO: 165 - protein, 17 aa, Gallus sp.
LISGKCSRFYLCCRIWG

SEQ ID NO: 166 - protein, 17 aa, Gallus sp.
LISGKCSRFHLCCKRIW

SEQ ID NO: 167 - protein, 18 aa, Meleagris gallopavo
VISGTCSRFQVCCKTLLG

SEQ ID NO: 168 - protein, 19 aa, Meleagris gallopavo
IKVGSCFGFRSCCKWPWDA

SEQ ID NO: 169 - protein, 19 aa, Gallus sp.
IKVGSCFGFRSCCKWPWNA

SEQ ID NO: 170 - protein, 19 aa, Oryctolagus sp.
VIDGSCGLFNSKYICCREK

SEQ ID NO: 171 - protein, 17 aa, Homo sapiens
IHVGRCLNSQPCCLPLG

SEQ ID NO: 172 - protein, 18 aa, Homo sapiens
YSYGTCTVMGINHRFCCL

SEQ ID NO: 173 - protein, 17 aa, Homo sapiens
YRIGRCPNTYACCLRKW

SEQ ID NO: 174 - protein, 22 aa, synthetic construct
  REGION 1..22: note = Description of Artificial Sequence: Synthetic consensus peptide
  VARIANT 1..3: note = variable amino acid
  VARIANT 5: note = variable amino acid
  VARIANT 7..14: note = variable amino acid
  VARIANT 16: note = variable amino acid
  VARIANT 18..22: note = variable amino acid
XXXGXCXXXXXXXXCXCXXXXX

SEQ ID NO: 175 - protein, 21 aa, Arabidopsis thaliana
ARHGSCNYVFPAHKCICYFPC

SEQ ID NO: 176 - protein, 21 aa, Brassica napus
ARHGSCNYVFPAHKCICYFPC

SEQ ID NO: 177 - protein, 21 aa, Sinapis alba
ARHGSCNYVFPAHKCICYFPC

SEQ ID NO: 178 - protein, 21 aa, Raphanus sativus
ARHGSCNYVFPAHKCICYFPC

SEQ ID NO: 179 - protein, 21 aa, Raphanus sativus
ARHGSCNYVFPAHKCICYFPC

SEQ ID NO: 180 - protein, 21 aa, Sinapis alba
ARHGSCNYVFPAHKCICYFPC

SEQ ID NO: 181 - protein, 21 aa, Arabidopsis thaliana
AKHGSCNYVFPAHKCICYFPC

SEQ ID NO: 182 - protein, 21 aa, Arabidopsis thaliana
AKHGSCNYVFPAHKCICYVPC

SEQ ID NO: 183 - protein, 21 aa, Arabidopsis thaliana
AKHGSCNYVFPAHKCICYVPC

SEQ ID NO: 184 - protein, 21 aa, Brassica napus
AQHGSCNYVFPAHKCICYFPC

SEQ ID NO: 185 - protein, 21 aa, Raphanus sativus
AQHGSCNYVFPAHKCICYFPC

SEQ ID NO: 186 - protein, 21 aa, Raphanus sativus
ARHGSCNYVFPYHRCICYFPC

SEQ ID NO: 187 - protein, 21 aa, Sinapis alba
SRHGSCNIPFPSNKCICYFPC

SEQ ID NO: 188 - protein, 21 aa, Aesculus hippocastanum
ASHGACHKRENHWKCFCYFNC

SEQ ID NO: 189 - protein, 19 aa, Nicotiana paniculata
FTDGKCSKILRRCICYKPC

SEQ ID NO: 190 - protein, 19 aa, Nicotiana tabacum
FTDGHCSKLLRRCICTKPC

SEQ ID NO: 191 - protein, 18 aa, Beta vulgaris
WPGGVCVPFLRCECQRSC

SEQ ID NO: 192 - protein, 18 aa, Beta vulgaris
WPNGKCLVGFKCECQRPC

SEQ ID NO: 193 - protein, 18 aa, Pisum sp.
AISGRCRDDFRCWCTKNC

SEQ ID NO: 194 - protein, 18 aa, Pisum sp.
LLSGRCRDDFRCWCTNRC

SEQ ID NO: 195 - protein, 16 aa, Drosophila melanogaster
RSSGHCSPSLKCWCEG

SEQ ID NO: 196 - protein, 19 aa, Capsicum annuum
FNGGHCRGFRRRCFCTRHC

SEQ ID NO: 197 - protein, 19 aa, Petunia integrifolia
FIGGNCRAFRRRCFCTRNC

SEQ ID NO: 198 - protein, 19 aa, Arabidopsis thaliana
FVGGNCRGFRRRCFCTRHC

SEQ ID NO: 199 - protein, 19 aa, Arabidopsis thaliana
FPGGDCRGFRRRCFCTRNC

SEQ ID NO: 200 - protein, 19 aa, Arabidopsis thaliana
FGGGKCRGFRRRCYCTRHC

SEQ ID NO: 201 - protein, 19 aa, Helianthus annuus
FSGGKCRGFRRRCFCTTHC

SEQ ID NO: 202 - protein, 19 aa, Vigna unguiculata
FSGGNCRGFRRRCFCTLKC

SEQ ID NO: 203 - protein, 20 aa, Capsicum annuum
FTDGSCIGFRLQCFCTKPCA

SEQ ID NO: 204 - protein, 17 aa, Vicia faba
YKGGDCHGLRRRCMCLC

SEQ ID NO: 205 - protein, 17 aa, Vicia faba
YKGGDCHGLRRRCMCLC

SEQ ID NO: 206 - protein, 19 aa, Spinacia oleracea
YPAGDCKGIRRRCMCSKPC

SEQ ID NO: 207 - protein, 19 aa, Triticum aestivum
WGGGNCDGPFRRCKCIRQC

SEQ ID NO: 208 - protein, 19 aa, Triticum aestivum
WGGGNCDGPFRRCKCIRQC

SEQ ID NO: 209 - protein, 19 aa, Hordeum vulgare
WGGGNCDGPFRRCKCMRRC

SEQ ID NO: 210 - protein, 19 aa, Zea mays
YGGGNCDGIMRQCKCIRQC

SEQ ID NO: 211 - protein, 16 aa, Protophormia terraenovae
NRGGYCNGKGVCVCRN

SEQ ID NO: 212 - protein, 16 aa, unidentified
  source 1..16: note = Sarcophaga peregrina
NRGGYCNGKAVCVCRN

SEQ ID NO: 213 - protein, 15 aa, Tenebrio molitor
RSGGYCNGKRVCVCR

SEQ ID NO: 214 - protein, 16 aa, unidentified
  source 1..16: note = Sarcophaga peregrina
NRGGYCTGNGICVCRN

SEQ ID NO: 215 - protein, 16 aa, Stomoxys calcitrans
DVGGYCTKEGLCVCKE

SEQ ID NO: 216 - protein, 16 aa, Aedes aegypti
NRGGYCNAKKVCVCRN

SEQ ID NO: 217 - protein, 16 aa, Anopheles gambiae
YRGGYCNSKAVCVCRN

SEQ ID NO: 218 - protein, 16 aa, Aedes aegypti
NRGGYCNSQKVCVCRN

SEQ ID NO: 219 - protein, 16 aa, unidentified
  source 1..16: note = Phlebotomus duboscqi
YRGGYCNSKAVCTCRR

SEQ ID NO: 220 - protein, 16 aa, Drosophila melanogaster
FKGGYCNDKAVCVCRN

SEQ ID NO: 221 - protein, 14 aa, unidentified
  source 1..14: note = Zophobas atratus
RKGGYCNSKSVCVC

SEQ ID NO: 222 - protein, 16 aa, Stomoxys calcitrans
KSGGRCNDDAVCVCRK

SEQ ID NO: 223 - protein, 18 aa, unidentified
  source 1..18: note = Androctonus australis
RRGGYCAGLFKQTCTCYR

SEQ ID NO: 224 - protein, 19 aa, unidentified
  source 1..19: note = Leiurus quinquestriatus hebraeus
RRGGYCAGFFKQTCTCYRN

SEQ ID NO: 225 - protein, 18 aa, unidentified
  source 1..18: note = Aeshna cyanea
RSGGYCSGPLKLTCTCYR

SEQ ID NO: 226 - protein, 15 aa, Mytilus edulis
RYGGYCGGHRLRCTC

SEQ ID NO: 227 - protein, 20 aa, Mytilus galloprovincialis
RCGGYCGGWHRLRCTCYRCG

SEQ ID NO: 228 - protein, 16 aa, Mytilus edulis
RGGYCGGHRLRCTCYR

SEQ ID NO: 229 - protein, 18 aa, Heliothis virescens
YKGGHCGSFANVNCWCEG

SEQ ID NO: 230 - protein, 18 aa, Brassica napus
GTVGSCAEEKGFCNCACK

SEQ ID NO: 231 - protein, 16 aa, Bombyx mori
AASGQCNPVCVEGCAC

SEQ ID NO: 232 - protein, 18 aa, Sus sp.
RGVCVCFRRRCYCLGGGR

SEQ ID NO: 233 - protein, 18 aa, Sus sp.
RGVCVCFRPRCYCLRGGR

SEQ ID NO: 234 - protein, 18 aa, Sus sp.
RGVCFCIWGRCYCLRGGR

SEQ ID NO: 235 - protein, 17 aa, Sus sp.
GVCICFRRRCYCLRGGR

SEQ ID NO: 236 - protein, 18 aa, Sus sp.
RGVCVCFRRRCYCLRGGR

SEQ ID NO: 237 - protein, 18 aa, Acanthoscurria gomesiana
RGRCYTVCRQKYCLRRCQ

SEQ ID NO: 238 - protein, 15 aa, Rana sp.
KQGACYAACKASKQC

SEQ ID NO: 239 - skipped

SEQ ID NO: 240 - protein, 13 aa, unidentified
  source 1..13: note = Androctonus australis
RRRGGCYYRCTNR

SEQ ID NO: 241 - protein, 10 aa, Leuconostoc gelidum
  VARIANT 2: note = variable amino acid
GXCTKSGCSV

SEQ ID NO: 242 - protein, 14 aa, unidentified
  source 1..14: note = Pseudacanthotermes spiniger
RRAFCDRSQCKCVF

SEQ ID NO: 243 - skipped

SEQ ID NO: 244 - protein, 21 aa, Mytilus edulis
RRCKGWASASFRGRCYCKCFR

SEQ ID NO: 245 - protein, 17 aa, Tachypleus tridentatus
RQCRGYTSGPFYSRCTL

SEQ ID NO: 246 - protein, 17 aa, Phytolacca americana
RNKCVGYSQGAIQFCYS

SEQ ID NO: 247 - protein, 12 aa, unidentified
  source 1..12: note = Aspergillus giganteus
CYCKGKYSDFEC

SEQ ID NO: 248 - protein, 13 aa, Podisus maculiventris
MRQCKGTRRNCYI

SEQ ID NO: 249 - protein, 13 aa, Macaca mulatta
ICRCVGRRCLCRC

SEQ ID NO: 250 - protein, 10 aa, Amaranthus caudatus
GCYGFQSCCM

SEQ ID NO: 251 - protein, 15 aa, Tachypleus tridentatus
RRYCIGRYCVRFCWK

SEQ ID NO: 252 - protein, 15 aa, Tachypleus tridentatus
RRYCIGRYCVRFCWK

SEQ ID NO: 253 - protein, 15 aa, Tachypleus gigas
RRYCIGRYCVRFCWK

SEQ ID NO: 254 - protein, 16 aa, Limulus polyphemus
RRYCFGRYCVRFCWKK

SEQ ID NO: 255 - protein, 16 aa, Limulus polyphemus
RRYCFGRYCVRFCWKK

SEQ ID NO: 256 - protein, 65 aa, unidentified
  source 1..65: note = Centruroides exilicauda
KEGYLVKKSDGCKYGCLKLGENEGCDTECKAKNQGGSYGYCYAFACWCEGLPESTPTYPLPNKSC

SEQ ID NO: 257 - protein, 77 aa, unidentified
  source 1..77: note = Hottentotta judaicus
MKKNGYPLDRNGKTTECSGVNAIAPHYCNSECTKVYYAESGYCCWGACYCFGLEDDKPIGPMKDITKKYCDVQIIPS

SEQ ID NO: 258 - protein, 64 aa, unidentified
  source 1..64: note = Mesobuthus martensii
VRDAYIAKPENCVYHCAGNEGCNKLCTDNGAESGYCQWGGRYGNACWCIKLPDDVPIRVPGKCH

SEQ ID NO: 259 - protein, 34 aa, unidentified
  source 1..34: note = Heterometrus spinifer
ASCRTPKDCADPCRKETGCPYGKCMNRKCKCNRC

SEQ ID NO: 260 - protein, 63 aa, unidentified
  source 1..63: note = Centruroides exilicauda
KKDGYPVDSGNCKYECLKDDYCNDLCLERKADKGYCYWGKVSCYCYGLPDNSPTKTSGKCNPA

SEQ ID NO: 261 - protein, 84 aa, unidentified
  source 1..84: note = Mesobuthus martensii
MNYLVMISFALLLMTGVESVRDAYIAKPHNCVYECARNEYCNDLCTKNGAKSGYCQWVGKYGNGCWCIELPDNVPIRVPGKCHR

SEQ ID NO: 262 - protein, 37 aa, unidentified
  source 1..37: note = Tityus serrulatus
VFINAKCRGSPECLPKCKEAIGKAAGKCMNGKCKCYP

SEQ ID NO: 263 - protein, 37 aa, synthetic construct
  VARIANT 1: note = variable amino acid
  source 1..37: note = Leiurus quinquestriatus hebraeus
XFTQESCTASNQCWSICKRLHNTNRGKCMNKKCRCYS

SEQ ID NO: 264 - protein, 68 aa, unidentified
  VARIANT 68: note = variable amino acid
  source 1..68: note = Leiurus quinquestriatus hebraeus
VRDGYIAQPENCVYHCFPGSSGCDTLCKEKGGTSGHCGFKVGHGLACWCNALPDNVGIIVEGEKCHSX

SEQ ID NO: 265 - protein, 38 aa, synthetic construct
  source 1..38: note = Androctonus mauretanicus mauretanicus
GVEINVKCSGSPQCLKPCKDAGMRFGKCMNRKCHCTPK

SEQ ID NO: 266 - protein, 37 aa, synthetic construct
  VARIANT 1: note = variable amino acid
  source 1..37: note = Mesobuthus martensii
XFTNVSCSASSQCWPVCKKLFGTYRGKCMNSKCRCYS

SEQ ID NO: 267 - protein, 35 aa, synthetic construct
VVIGQRCYRSPDCYSACKKLVGKATGKCTNGRCDC

SEQ ID NO: 268 - protein, 37 aa, unidentified
  VARIANT 1: note = variable amino acid
  source 1..37: note = Leiurus quinquestriatus hebraeus
XFTNVSCTTSKECWSVCQRLHNTSRGKCMNKKCRCYS

SEQ ID NO: 269 - protein, 67 aa, unidentified
  source 1..67: note = Leiurus quinquestriatus hebraeus
VRDGYIAQPENCVYHCFPGSSGCDTLCKEKGGTSGHCGFKVGHGLACWCNALPDNVGIIVEGEKCHS

SEQ ID NO: 270 - protein, 64 aa, unidentified
  source 1..64: note = Androctonus australis
VKDGYIVDDVNCTYFCGRNAYCNEECTKLKGESGYCQWASPYGNACYCYKLPDHVRTKGPGRCH

SEQ ID NO: 271 - protein, 38 aa, unidentified
  source 1..38: note = Leiurus quinquestriatus hebraeus
GVPINVSCTGSPQCIKPCKDAGMRFGKCMNRKCHCTPK

SEQ ID NO: 272 - protein, 64 aa, unidentified
  source 1..64: note = Mesobuthus martensii
GRDAYIADSENCTYFCGSNPYCNDVCTENGAKSGYCQWAGRYGNACYCIDLPASERIKEGGRCG

SEQ ID NO: 273 - protein, 57 aa, unidentified
  source 1..57: note = Mesobuthus eupeus
MKISFVLLLTLFICSIGWSEARPTDIKCSESYQCFPVCKSRFGKTNGRCVNGFCDCF

SEQ ID NO: 274 - protein, 35 aa, unidentified
  source 1..35: note = Pandinus imperator
TISCTNPKQCYPHCKKETGYPNAKCMNRKCKCFGR

SEQ ID NO: 275 - protein, 59 aa, unidentified
  source 1..59: note = Centruroides sculpturatus
KDGYPVDSKGCKLSCVANNYCDNQCKMKKASGGHCYAMSCYCEGLPENAKVSDSATNIC

SEQ ID NO: 276 - protein, 86 aa, unidentified
  source 1..86: note = Centruroides sculpturatus
MNSLLMITACLVLIGTVWAKDGYLVEKTGCKKTCYKLGENDFCNRECKWKHIGGSYGYCYGFGCYCEGLPDSTQTWPLPNKTCGKK

SEQ ID NO: 277 - protein, 78 aa, unidentified
  source 1..78: note = Conus textile
MKLTCMMIVAVLFLTAWTFATADDPRNGLGNLFSNAHHEMKNPEASKLNKRWCKQSGEMCNLLDQNCCDGYCIVLVCT

SEQ ID NO: 278 - protein, 49 aa, unidentified
  source 1..49: note = Anthopleura xanthogrammica
GVPCLCDSDGPRPRGNTLSGILWFYPSGCPSGWHNCKAHGPNIGWCCKK

SEQ ID NO: 279 - protein, 84 aa, unidentified
  source 1..84: note = Tityus serrulatus
MKGMILFISCLLLIGIVVECKEGYLMDHEGCKLSCFIRPSGYCGRECGIKKGSSGYCAWPACYCYGLPNWVKVWDRATNKCGKK

SEQ ID NO: 280 - protein, 38 aa, unidentified
  source 1..38: note = Hadronyche infensa
KCLAEAADCSPWSGDSCCKPYLCSCIFFYPCSCRPKGW

SEQ ID NO: 281 - protein, 35 aa, unidentified
  source 1..35: note = Ornithoctonus huwena
ECLEIFKACNPSNDQCCKSSKLVCSRKTRWCKYQI

SEQ ID NO: 282 - protein, 66 aa, unidentified
  source 1..66: note = Mesobuthus martensii
VRDGYIALPHNCAYGCLNNEYCNNLCTKDGAKIGYCNIVGKYGNACWCIQLPDNVPIRVPGRCHPA

SEQ ID NO: 283 - protein, 38 aa, unidentified
IEAIRCGGSRDCYRPCQKRTGCPNAKCINKTCKCYGCS

SEQ ID NO: 284 - protein, 42 aa, unidentified
DRDSCVDKSRCAKYGYYQECQDCCKNAGHNGGTCMFFKCKCA

SEQ ID NO: 285 - protein, 36 aa, unidentified
DCVRFWGKCSQTSDCCPHLACKSKWPRNICVWDGSV

SEQ ID NO: 286 - protein, 40 aa, unidentified
WCSTCLDLACGASRECYDPCFKAFGRAHGKCMNNKCRCYT

SEQ ID NO: 287 - protein, 35 aa, unidentified; identical to SEQ ID NO: 274

SEQ ID NO: 288 - protein, 39 aa, unidentified
TVIDVKCTSPKQCLPPCKAQFGIRAGAKCMNGKCKCYPH

SEQ ID NO: 289 - protein, 31 aa, unidentified
AACYSSDCRVKCVAMGFSSGKCINSKCKCYK

SEQ ID NO: 290 - protein, 37 aa, unidentified
  VARIANT 1: note = variable amino acid
XFTDVKCTGSKQCWPVCKQMFGKPNGKCMNGKCRCYS

SEQ ID NO: 291 - protein, 68 aa, unidentified
  VARIANT 1: note = variable amino acid
XPEIEAQGNECLKEYGGDVGFGFCAPRIFPTICYTRCRENKGAKGGRCRWGQGSNVKCLCDFCGDTPQ

SEQ ID NO: 292 - protein, 29 aa, unidentified
RVCPRILLECKKDSDCLAECVCLEHGYCG

SEQ ID NO: 293 - protein, 55 aa, unidentified
AHMDCTEFNPLCRCNKMLGDLICAVIGDAKEEHRNMCALCCEHPGGFEYSNGPCE

SEQ ID NO: 294 - protein, 32 aa, unidentified
CIPKWNRCGPKMDGVPCCEPYTCTSDYYGNCS

SEQ ID NO: 295 - protein, 28 aa, unidentified
GCPRILMRCKQDSDCLAGCVCGPNGFCG

SEQ ID NO: 296 - protein, 29 aa, unidentified
RVCPRILMECKKDSDCLAECVCLEHGYCG

SEQ ID NO: 297 - protein, 34 aa, unidentified
  VARIANT 1: note = variable amino acid
XGSDGGVCPKILKKCRRDSDCPGACICRGNGYCG

SEQ ID NO: 298 - protein, 50 aa, unidentified
NLMKRCTRGFRKLGKCTTLEEEKCKTLYPRGQCTCSDSKMNTHSCDCKSC

SEQ ID NO: 299 - protein, 37 aa, unidentified
ADCNGACSPFEVPPCRSRDCRCVPIGLFVGFCIHPTG

SEQ ID NO: 300 - protein, 46 aa, unidentified
KSCCRNTLARNCYNACRFTGGSQPTCGILCDCIHVTTTTCPSSHPS

SEQ ID NO: 301 - protein, 41 aa, unidentified
  VARIANT 1: note = variable amino acid
XTTGPCCRQCKLKPAGTTCWKTSLTSHYCTGKSCDCPLYPG

SEQ ID NO: 302 - protein, 50 aa, unidentified
RKGHFSRCPKQYKHYCIKGRCRFVVAEQTPSCVCDEGYIGARCERVDLFY

SEQ ID NO: 303 - protein, 29 aa, unidentified
  VARIANT 1: note = variable amino acid
XGLPVCGETCVGGTCNTPGCTCSWPVCTR

SEQ ID NO: 304 - protein, 23 aa, unidentified
  VARIANT 1: note = variable amino acid
XNFNGGCLAGYMRTADGRCKPTF

SEQ ID NO: 305 - protein, 25 aa, unidentified
ENFSGGCVAGYMRTPDGRCKPTFYQ

SEQ ID NO: 306 - protein, 58 aa, unidentified
PIEVNDDCMACEACVEICPDVFEMNEEGDKAVVINPDSDLDCVEEAIDSCPAEAIVRS

SEQ ID NO: 307 - protein, 32 aa, unidentified
  VARIANT 1: note = variable amino acid
XCLGDKCDYNNGCCSGYVCSRTWKWCVLAGPW

SEQ ID NO: 308 - protein, 55 aa, unidentified
TQGNTCGGETCSAAQVCLKGKCVCNEVHCRIRCKYGLKKDENGCEYPCSCAKASQ

SEQ ID NO: 309 - protein, 54 aa, unidentified
  VARIANT 1: note = variable amino acid
  source 1..54: note = Pentadiplandra brazzeana
XDKCKKVYENYPVSKCQLANQCNYDCKLDKHARSGECFYDEKRNLQCICDYCEY

SEQ ID NO: 310 - protein, 46 aa, unidentified
TTCCPSIVARSNFNVCRLPGTSEAICATYTGCIIIPGATCPGDYAN

SEQ ID NO: 311 - protein, 51 aa, unidentified
  VARIANT 1: note = variable amino acid
XVYTDCTESGQNLCLCEGSNVCGQGNKCILGSDGEKNQCVTGEGTPKPQSH

SEQ ID NO: 312 - protein, 13 aa, unidentified
TCEICAYAACTGC

SEQ ID NO: 313 - protein, 53 aa, unidentified
NSYPGCPSSYDGYCLNGGVCMHIESLDSYTCNCVIGYSGDRCQTRDLRWWELR

SEQ ID NO: 314 - protein, 16 aa, unidentified
NDDCELCVNVACTGCL

SEQ ID NO: 315 - protein, 43 aa, unidentified
EQCGRQAGGKLCPNNLCCSQWGWCGSTDEYCSPDHNCQSNCKD

SEQ ID NO: 316 - protein, 70 aa, Homo sapiens
GSEVSDKRTCVSLTTQRLPVSRIKTYTITEGSLRAVIFITKRGLKVCADPQATWVRDVVRSMDRKSNTRN

SEQ ID NO: 317 - protein, 72 aa, Homo sapiens
ASVATELRCQCLQTLQGIHPKNIQSVNVKSPGPHCAQTEVIATLKNGRKACLNPASPIVKKIIEKMLNSDKS

SEQ ID NO: 318 - protein, 71 aa, Homo sapiens
LATELRCQCLQTLQGIHLKNIQSVKVKSPGPHCAQTEVIATLKNGQKACLNPASPMVKKIIEKMLKNGKSN

SEQ ID NO: 319 - protein, 70 aa, Homo sapiens
EAEEDGDLQCLCVKTTSQVRPRHITSLEVIKAGPHCPTAQLIATLKNGRKICLDLQAPLYKKIIKKLLES

SEQ ID NO: 320 - protein, 73 aa, Homo sapiens
AVLRELRCVCLQTTQGVHPKMISNLQVFAIGPQCSKVEVVASLKNGKEICLDPEAPFLKKVIQKILDGGNKEN

SEQ ID NO: 321 - protein, 73 aa, Homo sapiens
AVLTELRCTCLRVTLRVNPKTIGKLQVFPAGPQCSKVEVVASLKNGKQVCLDPEAPFLKKVIQKILDSGNKKN

SEQ ID NO: 322 - protein, 73 aa, Homo sapiens
DLYAELRCMCIKTTSGIHPKNIQSLEVIGKGTHCNQVEVIATLKDGRKICLDPDAPRIKKIVQKKLAGDESAD

SEQ ID NO: 323 - protein, 72 aa, Homo sapiens
SAKELRCQCIKTYSKPFHPKFIKELRVIESGPHCANTEIIVKLSDGRELCLDPKENWVQRVVEKFLKRAENS

SEQ ID NO: 324 - protein, 72 aa, Homo sapiens
GRCSCISTNQGTIHLQSLKDLKQFAPSPSCEKIEIIATLKNGVQTCLNPDSADVKELIKKWEKQVSQKKKQK

SEQ ID NO: 325 - protein, 71 aa, Homo sapiens
VRCTCISISNQPVNPRSLEKLEIIPASQFCPRVEIIATMKKKGEKRCLNPESKAIKNLLKAVSKERSKRSP

SEQ ID NO: 326 - protein, 67 aa, Homo sapiens
GRCLCIGPGVKAVKVADIEKASIMYPSNNCDKIEVIITLKENKGQRCLNPKSKQARLIIKKVERKNF

SEQ ID NO: 327 - protein, 66 aa, Homo sapiens
YRCPCRFFESHVARANVKHLKILNTPNCALQIVARLKNNNRQVCIDPKLKWIQEYLEKALNKRFKM

SEQ ID NO: 328 - protein, 72 aa, Homo sapiens
LRCRCVQESSVFIPRRFIDRIQILPRGNGCPRKEIIVWKKNKSIVCVDPQAEWIQRMMEVLRKRSSSTLPVP

SEQ ID NO: 329 - protein, 67 aa, Homo sapiens
FSRCCFSFAEQEIPLRAILCYRNTSSICSNEGLIFKLKRGKEACALDTVGWVQRHRKMLRHCPSKRK

SEQ ID NO: 330 - protein, 69 aa, Homo sapiens
PITCCFNVINRKIPIQRLESYTRITNIQCPKEAVIFKTKRGKEVCADPKERWVRDSMKHLDQIFQNLKP

SEQ ID NO: 331 - protein, 68 aa, Homo sapiens
PSTCCFTFSSKKISLQRLKSYVITTSRCPQKAVIFRTKLGKEICADPKEKWVQNYMKHLGRKAHTLKT

SEQ ID NO: 332 - protein, 61 aa, Homo sapiens
SECCFTYTTYKIPRQRIMDYYETNSQCSKPGIVFITKRGHSVCTNRSDKWVQDYIKDMKEN

SEQ ID NO: 333 - protein, 67 aa, Homo sapiens
FHFAADCCTSYISQSIPCSLMKSYFETSSECSKPGVIFLTKKGRQVCAKPSGPGVQDCMKKLKPYSI

SEQ ID NO: 334 - protein, 64 aa, Homo sapiens
RECCLEYFKGAIPLRKLKTWYQTSEDCSRDAIVFVTVQGRAICSDPNNKRVKNAVKYLQSLERS

SEQ ID NO: 335 - protein, 62 aa, Homo sapiens
ELCCLVYTSWQIPQKFIVDYSETSPQCPKPGVILLTKRGRQICADPNKKWVQKYISDLKLNA

SEQ ID NO: 336 - protein, 72 aa, Homo sapiens
EDCCLSVTQKPIPGYIVRNFHYLLIKDGCRVPAVVFTTLRGRQLCAPPDQPWVERIIQRLQRTSAKMKRRSS

SEQ ID NO: 337 - protein, 74 aa, Homo sapiens
FHATSADCCISYTPRSIPCSLLESYFETNSECSKPGVIFLTKKGRRFCANPSDKQVQVCVRMLKLDTRIKTRKN

SEQ ID NO: 338 - protein, 70 aa, Homo sapiens
SPCCMFFVSKRIPENRVVSYQLSSRSTCLKAGVIFTTKKGQQFCGDPKQEWVQRYMKNLDAKQKKASPRA

SEQ ID NO: 339 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 340 - protein, 62 aa, Homo sapiens
TACCFSYTSRQIPQNFIADYFETSSQCSKPGVIFLTKRSRQVCADPSEEWVQKYVSDLELSA

SEQ ID NO: 341 - protein, 61 aa, Homo sapiens
TACCFSYTARKLPRNFVVDYYETSSLCSQPAVVFQTKRSKQVCADPSESWVQEYVYDLELN

SEQ ID NO: 342 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 343 - protein, 69 aa, Homo sapiens
STTCCYRFINKKIPKQRLESYRRTTSSHCPREAVIFKTKLDKEICADPTQKWVQDFMKHLDKKTQTPKL

SEQ ID NO: 344 - protein, 69 aa, Homo sapiens
PTTCCFNLANRKIPLQRLESYRRITSGKCPQKAVIFKTKLAKDICADPKKKWVQDSMKYLDQKSPTPKP

SEQ ID NO: 345 - protein, 68 aa, Homo sapiens
STCCLKYYEKVLPRRLVVGYRKALNCHLPAIIFVTKRNREVCTNPNDDWVQEYIKDPNLPLLPTRNLS

SEQ ID NO: 346 - protein, 67 aa, Homo sapiens
FDCCLGYTDRILHPKFIVGFTRQLANEGCDINAIIFHTKKKLSVCANPKQTWVKYIVRLLSKKVKNM

SEQ ID NO: 347 - protein, 73 aa, Homo sapiens
QDCCLKYSQRKIPAKVVRSYRKQEPSLGCSIPAILFLPRKRSQAELCADPKELWVQQLMQHLDKTPSPQKPAQ

SEQ ID NO: 348 - protein, 60 aa, Homo sapiens
SVCCRDYVRYRLPLRVVKHFYWTSDSCPRPGVVLLTFRDKEICADPRVPWVKMILNKLSQ

SEQ ID NO: 349 - protein, 75 aa, Homo sapiens
FEDCCLAYHYPIGWAVLRRAWTYRIQEVSGSCNLPAAIFYLPKRHRKVCGNPKSREVQRAMKLLDARNKVFAKLH

SEQ ID NO: 350 - protein, 73 aa, Homo sapiens
TACCTQLYRKPLSDKLLRKVIQVELQEADGDCHLQAFVLHLAQRSICIHPQNPSLSQWFEHQERKLHGTLPKL

SEQ ID NO: 351 - protein, 72 aa, Homo sapiens
SSCCTEVSHHISRRLLERVNMCRIQRADGDCDLAAVILHVKRRRICVSPHNHTVKQWMKVQAAKKNGKGNVC

SEQ ID NO: 352 - protein, 19 aa, Aesculus hippocastanum
HGACHKRENHWKCFCYFNC

SEQ ID NO: 353 - protein, 20 aa, Homo sapiens
CANTEIIVKLSDGRELCLDP

SEQ ID NO: 354 - protein, 23 aa, Homo sapiens
CPTAQLIATLKNGRKICLDLQAP

SEQ ID NO: 355 - protein, 23 aa, Homo sapiens
CAQTEVIATLKNGRKACLNPASP

SEQ ID NO: 356 - protein, 17 aa, Homo sapiens
RAVIFITKRGLKVCADP

SEQ ID NO: 357 - protein, 20 aa, Homo sapiens
CPKEAVIFKTIVAKEICADP

SEQ ID NO: 358 - protein, 20 aa, Homo sapiens
CSNPAVVFVTRKNRQVCANP

SEQ ID NO: 359 - protein, 27 aa, synthetic construct
  REGION 1..27: note = Description of Artificial Seqeunce: Synthetic peptide
DSADVKELIKKWEKQVSQKKKQKNGKK

SEQ ID NO: 360 - protein, 18 aa, synthetic construct
  VARIANT 1..3: note = Variable amino acid and this region may encompass 1 to 3 residues
  VARIANT 5: note = Variable amino acid
  VARIANT 7..15: note = Variable amino acid and this region may encompass 2 to 9 residues
  VARIANT 17: note = Variable amino acid that may or may not be present
XXXGXCXXXXXXXXXCXC

SEQ ID NO: 361 - protein, 22 aa, synthetic construct
  VARIANT 2..14: note = region may encompass between 10 and 13 variable amino acids
  VARIANT 16..18: note = variable amino acid
  VARIANT 20..21: note = variable amino acid
CXXXXXXXXXXXXXGXXXCXXP